(19) United States
(12) Patent Application Publication
(10) Pub. No.: US 2023/0290440 A1
(43) Pub. Date: Sep. 14, 2023

(54) UROTHELIAL TUMOR MICROENVIRONMENT (TME) TYPES

(71) Applicant: BostonGene Corporation, Waltham, MA (US)

(72) Inventors: Natalia Miheecheva, Yerevan (AM); Konstantin Chernyshov, Yerevan (AM); Aleksandr Vikhorev, Moscow (RU)

(73) Assignee: BostonGene Corporation, Waltham, MA (US)

(21) Appl. No.: 18/168,721

(22) Filed: Feb. 14, 2023

Related U.S. Application Data
(60) Provisional application No. 63/310,057, filed on Feb. 14, 2022.

(51) Int. Cl. G16B 25/00; G16B 20/50; G16B 30/10; A61K 47/68; G16B 40/20

(52) U.S. Cl. CPC G16B 25/00; A61K 47/6803; G16B 20/50; G16B 30/10; G16B 40/20

(57) ABSTRACT

Aspects of the disclosure relate to methods, systems, computer-readable storage media, and graphical user interfaces (GUIs) that are useful for characterizing subjects having certain cancers, for example bladder cancers or urothelial cancers. The disclosure is based, in part, on methods for determining the urothelial cancer (UC) tumor microenvironment (TME) type of a urothelial cancer subject and the subject’s prognosis and/or likelihood of responding to a therapy based upon the UC TME type determination.

### RELATED APPLICATIONS

This Application claims the benefit under 35 U.S.C. § 119(e) of the filing date of U.S. Provisional Application Serial No. 63/310,057, filed Feb. 14, 2022, entitled “UROTHELIAL TRANSCRIPTOMIC AND MUTATIONAL SUBTYPES,” the entire contents of which are herein incorporated by reference.

### BACKGROUND

Bladder cancer (BLCA) is the tenth most common cancer worldwide, with urothelial carcinoma (UC) as the predominant histological subtype, which is characterized by high recurrence rates, progression, and resistance to platinum-based therapy. Although several immune checkpoint inhibitors (ICIs) have recently appeared in treatment strategy, the response rate is only ~15-25%.

### SUMMARY

Aspects of the disclosure relate to methods systems, and computer-readable storage media, which are useful for characterizing subjects having bladder cancers or urothelial cancers. The disclosure is based, in part, on methods for identifying the tumor microenvironment (TME) of a subject having urothelial cancer by using gene expression data obtained from the subject to produce a urothelial cancer (UC) signature that, when processed by methods disclosed herein, allows for assignment of a UC type to the subject. In some embodiments, the UC type of a subject is indicative of one or more characteristics of the subject (or the subject’s cancer), for example the likelihood a subject will have a good prognosis or respond to a therapeutic agent such as an immunotherapy (e.g., an immune checkpoint inhibitor), anti-FGFR agent, etc.).

Accordingly, in some aspects the disclosure provides a method for determining a urothelial cancer (UC) tumor microenvironment (TME) type of a subject having, suspected of having, or at risk of having a urothelial cancer, the method comprising using at least one computer hardware processor to perform obtaining RNA expression data for the subject, the RNA expression data indicating RNA expression levels for at least some genes in each group of at least some of a plurality of gene groups listed in Table 1; generating a UC TME signature for the subject using the RNA expression data, the UC TME signature comprising gene group scores for respective gene groups in the plurality of gene groups, the generating comprising determining the UC TME signature by determining the gene group scores using the RNA expression levels; and identifying, using the UC TME signature and from among a plurality of UC TME types, a UC TME type for the subject.

In some embodiments, obtaining the RNA expression data for the subject comprises obtaining sequencing data previously obtained by sequencing a biological sample obtained from the subject. In some embodiments, sequencing data comprises at least 1 million reads, at least 5 million reads, at least 10 million reads, at least 20 million reads, at least 50 million reads, or at least 100 million reads.

In some embodiments, sequencing data comprises whole exome sequencing (WES) data, bulk RNA sequencing (RNA-seq) data, single cell RNA sequencing (scRNA-seq) data, or next generation sequencing (NGS) data. In some embodiments, sequencing data comprises microarray data.

In some embodiments, generating a UC TME signature further comprises normalizing the RNA expression data to transcripts per million (TPM) units prior to generating the UC TME signature.

In some embodiments, obtaining the RNA expression data for a subject comprises sequencing a biological sample obtained from a subject. In some embodiments, a biological sample comprises urothelial tissue of a subject. In some embodiments, a biological sample comprises tumor tissue of a subject.

In some embodiments, the RNA expression levels comprise RNA expression levels for at least three genes from each of at least two of the following gene groups:
(i) Luminal differentiation group: PWRN1, PWRN3, GSTM5, GSTM4, GSTM2, ZNF321P, ZNF320, ZNF66, ZNF737, KRT20, UPK1B, FOXA1, ACER2, SEMA5A, PPARG, GATA3, SNX31, UPK2, UPK1A;
(ii) Basal differentiation group: TM4SF19, SERPINB13, SERPINB3, SERPINB4, SPRR2F, SPRR2E, SPRR2A, SPRR2D, KRT17, KRT16, KRT14, DSG3, KRT5, KRT6C, KRT6A, KRT6B;
(iii) Neuroendocrine differentiation group: PLEKHG4B, GNG4, PEG10, SOX2, TUBB2B, CHGB, SYP, ENO2, SV2A, MSI1, RND2, APLP1; and
(iv) FGFR3 co-expressed group: FGFR3, TP63, IRS1, WNT7B, CAPNS2, ZNF385A, SMAD3, SLC2A9, DUOXA1, SYTL1, SEMA4B, CLCA4, PLCH2, SSH3, PTPN13, TMPRSS4.

In some embodiments, the RNA expression levels further comprise RNA expression levels for at least three genes from each of at least two of the following gene groups:
(a) MHC type I group: HLA-C, TAPBP, HLA-B, B2M, TAP2, HLA-A, TAP1, NLRC5;
(b) MHC type II group: HLA-DQB1, HLA-DMA, HLA-DMB, HLA-DRA, CIITA, HLA-DQA1, HLA-DPB1, HLA-DRB1, HLA-DPA1;
(c) Coactivation molecules group: TNFRSF4, CD27, CD80, CD40LG, TNFRSF9, CD40, CD28, ICOSLG, CD83, TNFSF9, CD70, TNFSF4, ICOS, CD86;
(d) Effector cells group: ZAP70, GZMB, GZMK, IFNG, FASLG, EOMES, TBX21, GZMA, CD8A, GNLY, PRF1, CD8B;
(e) Natural killer cells group: NKG7, FGFBP2, CD244, KLRK1, KIR2DL4, CD226, KLRF1, GNLY, GZMB, KLRC2, NCR1, GZMH, IFNG, SH2D1B, NCR3, EOMES, CD160;
(f) T cells group: TRBC2, CD3E, CD3G, ITK, CD28, TRBC1, TRATI, TBX21, CD5, TRAC, CD3D;
(g) T-helper cells type 1 group: IL21, TBX21, IL12RB2, CD40LG, IFNG, IL2, STAT4;
(h) T-helper cells type 2 group: IL13, CCR4, IL10, IL4, IL5;
(i) B cells group: CD22, TNFRSF13C, STAP1, CD79B, PAX5, CR2, TNFRSF13B, CD79A, TNFRSF17, FCRL5, MS4A1, CD19, BLK;
(j) Macrophages group: MRC1, SIGLEC1, MSR1, CD163, CSF1R, CD68, IL4I1, IL10;
(k) Macrophages type 1 group: CMKLR1, SOCS3, IRF5, NOS2, IL1B, IL12B, IL23A, TNF, IL12A;
(l) Antitumor cytokines group: CCL3, IL21, IFNB1, IFNA2, TNF, TNFSF10;
(m) Checkpoint inhibition group: PDCD1, BTLA, HAVCR2, CD274, VSIR, LAG3, TIGIT, PDCD1LG2, CTLA4;
(n) T-regulatory cells group: IKZF2, TNFRSF18, IL10, FOXP3, CCR8, IKZF4, CTLA4;
(o) Neutrophils group: CD177, FFAR2, PGLYRP1, CXCR1, MPO, CXCR2, ELANE, CTSG, PRTN3, FCGR3B;
(p) MDSC group: ARG1, IL6, CYBB, IL10, PTGS2, IDO1, IL4I1;
(q) Protumor cytokines group: TGFB2, MIF, IL6, TGFB3, TGFB1, IL22, IL10;
(r) Cancer associated fibroblasts (CAF) group: COL6A3, PDGFRB, COL6A1, MFAP5, COL5A1, FAP, PDGFRA, FGF2, ACTA2, COL6A2, FBLN1, CD248, COL1A1, MMP2, COLIA2, MMP3, LUM, CXCL12, LRP1;
(s) Matrix group: LAMC2, TNC, COL11A1, VTN, LAMB3, COL1A1, FN1, LAMA3, LGALS9, COLIA2, COL4A1, COL5A1, ELN, LGALS7, COL3A1;
(t) Matrix remodeling group: ADAMTS4, ADAMTS5, CA9, LOX, MMP1, MMP11, MMP12, MMP2, MMP3, MMP7, MMP9, PLOD2;
(u) Angiogenesis group: VEGFC, VEGFA, PDGFC, KDR, CDH5, VEGFB, PGF, TEK, ANGPT2, CXCR2, FLT1, CXCL8, VWF, ANGPTI, CXCL5;
(v) Endothelium group: KDR, CDH5, NOS3, VCAM1, VWF, FLT1, MMRN1, CLEC14A, ENG, MMRN2;
(w) Proliferation rate group: CCND1, CCNB1, CETN3, CDK2, E2F1, AURKA, BUB1, AURKB, PLK1, MCM6, ESCO2, MYBL2, MKI67, MCM2, CCNE1; and
(x) Epithelial to mesenchymal transition group: CDH2, ZEB1, ZEB2, TWIST1, SNAI1, SNAI2, TWIST2.

In some embodiments, the RNA expression levels comprise RNA expression levels for each gene from each of the following gene groups:
(a) MHC type I group: HLA-C, TAPBP, HLA-B, B2M, TAP2, HLA-A, TAP1, NLRC5;
(b) MHC type II group: HLA-DQB1, HLA-DMA, HLA-DMB, HLA-DRA, CIITA, HLA-DQA1, HLA-DPB1, HLA-DRB1, HLA-DPA1;
(c) Coactivation molecules group: TNFRSF4, CD27, CD80, CD40LG, TNFRSF9, CD40, CD28, ICOSLG, CD83, TNFSF9, CD70, TNFSF4, ICOS, CD86;
(d) Effector cells group: ZAP70, GZMB, GZMK, IFNG, FASLG, EOMES, TBX21, GZMA, CD8A, GNLY, PRF1, CD8B;
(e) Natural killer cells group: NKG7, FGFBP2, CD244, KLRK1, KIR2DL4, CD226, KLRF1, GNLY, GZMB, KLRC2, NCR1, GZMH, IFNG, SH2D1B, NCR3, EOMES, CD160;
(f) T cells group: TRBC2, CD3E, CD3G, ITK, CD28, TRBC1, TRATI, TBX21, CD5, TRAC, CD3D;
(g) T-helper cells type 1 group: IL21, TBX21, IL12RB2, CD40LG, IFNG, IL2, STAT4;
(h) T-helper cells type 2 group: IL13, CCR4, IL10, IL4, IL5;
(i) B cells group: CD22, TNFRSF13C, STAP1, CD79B, PAX5, CR2, TNFRSF13B, CD79A, TNFRSF17, FCRL5, MS4A1, CD19, BLK;
(j) Macrophages group: MRC1, SIGLEC1, MSR1, CD163, CSF1R, CD68, IL4I1, IL10;
(k) Macrophages type 1 group: CMKLR1, SOCS3, IRF5, NOS2, IL1B, IL12B, IL23A, TNF, IL12A;
(l) Antitumor cytokines group: CCL3, IL21, IFNB1, TNF, TNFSF10;
(m) Checkpoint inhibition group: PDCD1, BTLA, HAVCR2, CD274, VSIR, LAG3, TIGIT, PDCD1LG2, CTLA4;
(n) T-regulatory cells group: IKZF2, TNFRSF18, IL10, FOXP3, CCR8, IKZF4, CTLA4;
(o) Neutrophils group: CD177, FFAR2, PGLYRP1, CXCR1, MPO, CXCR2, ELANE, CTSG, PRTN3, FCGR3B;
(p) MDSC group: ARG1, IL6, CYBB, IL10, PTGS2, IDO1, IL4I1;
(q) Protumor cytokines group: TGFB2, MIF, IL6, TGFB3, TGFB1, IL22, IL10;
(r) Cancer associated fibroblasts (CAF) group: COL6A3, PDGFRB, COL6A1, MFAP5, COL5A1, FAP, PDGFRA, FGF2, ACTA2, COL6A2, FBLN1, CD248, COL1A1, MMP2, COLIA2, MMP3, LUM, CXCL12, LRP1;
(s) Matrix group: LAMC2, TNC, COL11A1, VTN, LAMB3, COL1A1, FN1, LAMA3, LGALS9, COLIA2, COL4A1, COL5A1, ELN, LGALS7, COL3A1;
(t) Matrix remodeling group: ADAMTS4, ADAMTS5, CA9, LOX, MMP1, MMP11, MMP12, MMP2, MMP3, MMP7, MMP9, PLOD2;
(u) Angiogenesis group: VEGFC, VEGFA, PDGFC, KDR, CDH5, VEGFB, PGF, TEK, ANGPT2, CXCR2, FLT1, CXCL8, VWF, ANGPTI, CXCL5;
(v) Endothelium group: KDR, CDH5, NOS3, VCAM1, VWF, FLT1, MMRN1, CLEC14A, ENG, MMRN2;
(w) Proliferation rate group: CCND1, CCNB1, CETN3, CDK2, E2F1, AURKA, BUB1, AURKB, PLK1, MCM6, ESCO2, MYBL2, MKI67, MCM2, CCNE1;
(x) Epithelial to mesenchymal transition group: CDH2, ZEB1, ZEB2, TWIST1, SNAI1, SNAI2, TWIST2;
(y) Luminal differentiation group: PWRN1, PWRN3, GSTM5, GSTM4, GSTM2, ZNF321P, ZNF320, ZNF66, ZNF737, KRT20, UPK1B, FOXA1, ACER2, SEMA5A, PPARG, GATA3, SNX31, UPK2, UPK1A;
(z) Basal differentiation group: TM4SF19, SERPINB13, SERPINB3, SERPINB4, SPRR2F, SPRR2E, SPRR2A, SPRR2D, KRT17, KRT16, KRT14, DSG3, KRT5, KRT6C, KRT6A, KRT6B;
(aa) Neuroendocrine differentiation group: PLEKHG4B, GNG4, PEG10, SOX2, TUBB2B, CHGB, SYP, ENO2, SV2A, MSI1, RND2, APLP1; and
(bb) FGFR3 co-expressed group: FGFR3, TP63, IRS1, WNT7B, CAPNS2, ZNF385A, SMAD3, SLC2A9, DUOXA1, SYTL1, SEMA4B, CLCA4, PLCH2, SSH3, PTPN13, TMPRSS4.

In some embodiments, determining the gene group scores comprises determining a respective gene group score for each of at least two of the following gene groups, using, for a particular gene group, RNA expression levels for at least three genes in the particular gene group to determine the gene group score for the particular group, the gene groups including:
(i) Luminal differentiation group: PWRN1, PWRN3, GSTM5, GSTM4, GSTM2, ZNF321P, ZNF320, ZNF66, ZNF737, KRT20, UPK1B, FOXA1, ACER2, SEMA5A, PPARG, GATA3, SNX31, UPK2, UPK1A;
(ii) Basal differentiation group: TM4SF19, SERPINB13, SERPINB3, SERPINB4, SPRR2F, SPRR2E, SPRR2A, SPRR2D, KRT17, KRT16, KRT14, DSG3, KRT5, KRT6C, KRT6A, KRT6B;
(iii) Neuroendocrine differentiation group: PLEKHG4B, GNG4, PEG10, SOX2, TUBB2B, CHGB, SYP, ENO2, SV2A, MSI1, RND2, APLP1; and
(iv) FGFR3 co-expressed group: FGFR3, TP63, IRS1, WNT7B, CAPNS2, ZNF385A, SMAD3, SLC2A9, DUOXA1, SYTL1, SEMA4B, CLCA4, PLCH2, SSH3, PTPN13, TMPRSS4.

In some embodiments, determining the gene group scores further comprises determining a respective gene group score for each of at least two of the following gene groups, using, for a particular gene group, RNA expression levels for at least three genes in the particular gene group to determine the gene group score for the particular group, the gene groups including:
(a) MHC type I group: HLA-C, TAPBP, HLA-B, B2M, TAP2, HLA-A, TAP1, NLRC5;
(b) MHC type II group: HLA-DQB1, HLA-DMA, HLA-DMB, HLA-DRA, CIITA, HLA-DQA1, HLA-DPB1, HLA-DRB1, HLA-DPA1;
(c) Coactivation molecules group: TNFRSF4, CD27, CD80, CD40LG, TNFRSF9, CD40, CD28, ICOSLG, CD83, TNFSF9, CD70, TNFSF4, ICOS, CD86;
(d) Effector cells group: ZAP70, GZMB, GZMK, IFNG, FASLG, EOMES, TBX21, GZMA, CD8A, GNLY, PRF1, CD8B;
(e) Natural killer cells group: NKG7, FGFBP2, CD244, KLRK1, KIR2DL4, CD226, KLRF1, GNLY, GZMB, KLRC2, NCR1, GZMH, IFNG, SH2D1B, NCR3, EOMES, CD160;
(f) T cells group: TRBC2, CD3E, CD3G, ITK, CD28, TRBC1, TRATI, TBX21, CD5, TRAC, CD3D;
(g) T-helper cells type 1 group: IL21, TBX21, IL12RB2, CD40LG, IFNG, IL2, STAT4;
(h) T-helper cells type 2 group: IL13, CCR4, IL10, IL4, IL5;
(i) B cells group: CD22, TNFRSF13C, STAP1, CD79B, PAX5, CR2, TNFRSF13B, CD79A, TNFRSF17, FCRL5, MS4A1, CD19, BLK;
(j) Macrophages group: MRC1, SIGLEC1, MSR1, CD163, CSF1R, CD68, IL4I1, IL10;
(k) Macrophages type 1 group: CMKLR1, SOCS3, IRF5, NOS2, IL1B, IL12B, IL23A, TNF, IL12A;
(l) Antitumor cytokines group: CCL3, IL21, IFNB1, IFNA2, TNF, TNFSF10;
(m) Checkpoint inhibition group: PDCD1, BTLA, HAVCR2, CD274, VSIR, LAG3, TIGIT, PDCD1LG2, CTLA4;
(n) T-regulatory cells group: IKZF2, TNFRSF18, IL10, FOXP3, CCR8, IKZF4, CTLA4;
(o) Neutrophils group: CD177, FFAR2, PGLYRP1, CXCR1, MPO, CXCR2, ELANE, CTSG, PRTN3, FCGR3B;
(p) MDSC group: ARG1, IL6, CYBB, IL10, PTGS2, IDO1, IL4I1;
(q) Protumor cytokines group: TGFB2, MIF, IL6, TGFB3, TGFB1, IL22, IL10;
(r) Cancer associated fibroblasts (CAF) group: COL6A3, PDGFRB, COL6A1, MFAP5, COL5A1, FAP, PDGFRA, FGF2, ACTA2, COL6A2, FBLN1, CD248, COL1A1, MMP2, COLIA2, MMP3, LUM, CXCL12, LRP1;
(s) Matrix group: LAMC2, TNC, COL11A1, VTN, LAMB3, COL1A1, FN1, LAMA3, LGALS9, COLIA2, COL4A1, COL5A1, ELN, LGALS7, COL3A1;
(t) Matrix remodeling group: ADAMTS4, ADAMTS5, CA9, LOX, MMP1, MMP11, MMP12, MMP2, MMP3, MMP7, MMP9, PLOD2;
(u) Angiogenesis group: VEGFC, VEGFA, PDGFC, KDR, CDH5, VEGFB, PGF, TEK, ANGPT2, CXCR2, FLT1, CXCL8, VWF, ANGPTI, CXCL5;
(v) Endothelium group: KDR, CDH5, NOS3, VCAM1, VWF, FLT1, MMRN1, CLEC14A, ENG, MMRN2;
(w) Proliferation rate group: CCND1, CCNB1, CETN3, CDK2, E2F1, AURKA, BUB1, AURKB, PLK1, MCM6, ESCO2, MYBL2, MKI67, MCM2, CCNE1; and
(x) Epithelial to mesenchymal transition group: CDH2, ZEB1, ZEB2, TWIST1, SNAI1, SNAI2, TWIST2.

In some embodiments, determining the gene group scores comprises determining a respective gene group score for each of the following gene groups, using, for a particular gene group, RNA expression levels for all genes in the particular gene group to determine the gene group score for the particular group, the gene groups including:
(i) Luminal differentiation group: PWRN1, PWRN3, GSTM5, GSTM4, GSTM2, ZNF321P, ZNF320, ZNF66, ZNF737, KRT20, UPK1B, FOXA1, ACER2, SEMA5A, PPARG, GATA3, SNX31, UPK2, UPK1A;
(ii) Basal differentiation group: TM4SF19, SERPINB13, SERPINB3, SERPINB4, SPRR2F, SPRR2E, SPRR2A, SPRR2D, KRT17, KRT16, KRT14, DSG3, KRT5, KRT6C, KRT6A, KRT6B;
(iii) Neuroendocrine differentiation group: PLEKHG4B, GNG4, PEG10, SOX2, TUBB2B, CHGB, SYP, ENO2, SV2A, MSI1, RND2, APLP1; and
(iv) FGFR3 co-expressed group: FGFR3, TP63, IRS1, WNT7B, CAPNS2, ZNF385A, SMAD3, SLC2A9, DUOXA1, SYTL1, SEMA4B, CLCA4, PLCH2, SSH3, PTPN13, TMPRSS4.

In some embodiments, determining the gene group scores further comprises determining a respective gene group score for each of following gene groups, using, for a particular gene group, RNA expression levels for all genes in the particular gene group to determine the gene group score for the particular group, the gene groups including:
(a) MHC type I group: HLA-C, TAPBP, HLA-B, B2M, TAP2, HLA-A, TAP1, NLRC5;
(b) MHC type II group: HLA-DQB1, HLA-DMA, HLA-DMB, HLA-DRA, CIITA, HLA-DQA1, HLA-DPB1, HLA-DRB1, HLA-DPA1;
(c) Coactivation molecules group: TNFRSF4, CD27, CD80, CD40LG, TNFRSF9, CD40, CD28, ICOSLG, CD83, TNFSF9, CD70, TNFSF4, ICOS, CD86;
(d) Effector cells group: ZAP70, GZMB, GZMK, IFNG, FASLG, EOMES, TBX21, GZMA, CD8A, GNLY, PRF1, CD8B;
(e) Natural killer cells group: NKG7, FGFBP2, CD244, KLRK1, KIR2DL4, CD226, KLRF1, GNLY, GZMB, KLRC2, NCR1, GZMH, IFNG, SH2D1B, NCR3, EOMES, CD160;
(f) T cells group: TRBC2, CD3E, CD3G, ITK, CD28, TRBC1, TRATI, TBX21, CD5, TRAC, CD3D;
(g) T-helper cells type 1 group: IL21, TBX21, IL12RB2, CD40LG, IFNG, IL2, STAT4;
(h) T-helper cells type 2 group: IL13, CCR4, IL10, IL4, IL5;
(i) B cells group: CD22, TNFRSF13C, STAP1, CD79B, PAX5, CR2, TNFRSF13B, CD79A, TNFRSF17, FCRL5, MS4A1, CD19, BLK;
(j) Macrophages group: MRC1, SIGLEC1, MSR1, CD163, CSF1R, CD68, IL4I1, IL10;
(k) Macrophages type 1 group: CMKLR1, SOCS3, IRF5, NOS2, IL1B, IL12B, IL23A, TNF, IL12A;
(l) Antitumor cytokines group: CCL3, IL21, IFNB1, IFNA2, TNF, TNFSF10;
(m) Checkpoint inhibition group: PDCD1, BTLA, HAVCR2, CD274, VSIR, LAG3, TIGIT, PDCD1LG2, CTLA4;
(n) T-regulatory cells group: IKZF2, TNFRSF18, IL10, FOXP3, CCR8, IKZF4, CTLA4;
(o) Neutrophils group: CD177, FFAR2, PGLYRP1, CXCR1, MPO, CXCR2, ELANE, CTSG, PRTN3, FCGR3B;
(p) MDSC group: ARG1, IL6, CYBB, IL10, PTGS2, IDO1, IL4I1;
(q) Protumor cytokines group: TGFB2, MIF, IL6, TGFB3, TGFB1, IL22, IL10;
(r) Cancer associated fibroblasts (CAF) group: COL6A3, PDGFRB, COL6A1, MFAP5, COL5A1, FAP, PDGFRA, FGF2, ACTA2, COL6A2, FBLN1, CD248, COL1A1, MMP2, COLIA2, MMP3, LUM, CXCL12, LRP1;
(s) Matrix group: LAMC2, TNC, COL11A1, VTN, LAMB3, COL1A1, FN1, LAMA3, LGALS9, COL1A2, COL4A1, COL5A1, ELN, LGALS7, COL3A1;
(t) Matrix remodeling group: ADAMTS4, ADAMTS5, CA9, LOX, MMP1, MMP11, MMP12, MMP2, MMP3, MMP7, MMP9, PLOD2;
(u) Angiogenesis group: VEGFC, VEGFA, PDGFC, KDR, CDH5, VEGFB, PGF, TEK, ANGPT2, CXCR2, FLT1, CXCL8, VWF, ANGPT1, CXCL5;
(v) Endothelium group: KDR, CDH5, NOS3, VCAM1, VWF, FLT1, MMRN1, CLEC14A, ENG, MMRN2;
(w) Proliferation_rate group: CCND1, CCNB1, CETN3, CDK2, E2F1, AURKA, BUB1, AURKB, PLK1, MCM6, ESCO2, MYBL2, MKI67, MCM2, CCNE1; and
(x) Epithelial to mesenchymal transition group: CDH2, ZEB1, ZEB2, TWIST1, SNAI1, SNAI2, TWIST2.

In some embodiments, determining the gene group scores comprises determining a first score of a first gene group using a single-sample GSEA (ssGSEA) technique from RNA expression levels for at least some of the genes in one of the following gene groups:
(i) Luminal differentiation group: PWRN1, PWRN3, GSTM5, GSTM4, GSTM2, ZNF321P, ZNF320, ZNF66, ZNF737, KRT20, UPK1B, FOXA1, ACER2, SEMA5A, PPARG, GATA3, SNX31, UPK2, UPK1A;
(ii) Basal differentiation group: TM4SF19, SERPINB13, SERPINB3, SERPINB4, SPRR2F, SPRR2E, SPRR2A, SPRR2D, KRT17, KRT16, KRT14, DSG3, KRT5, KRT6C, KRT6A, KRT6B;
(iii) Neuroendocrine differentiation group: PLEKHG4B, GNG4, PEG10, SOX2, TUBB2B, CHGB, SYP, ENO2, SV2A, MSI1, RND2, APLP1; and
(iv) FGFR3 co-expressed group: FGFR3, TP63, IRS1, WNT7B, CAPNS2, ZNF385A, SMAD3, SLC2A9, DUOXA1, SYTL1, SEMA4B, CLCA4, PLCH2, SSH3, PTPN13, TMPRSS4.

In some embodiments, determining the gene group scores comprises determining gene group scores of one or more additional gene groups using a single-sample GSEA (ssGSEA) technique from RNA expression levels for at least some of the genes in one of the following gene groups:
(a) MHC type I group: HLA-C, TAPBP, HLA-B, B2M, TAP2, HLA-A, TAP1, NLRC5;
(b) MHC type II group: HLA-DQB1, HLA-DMA, HLA-DMB, HLA-DRA, CIITA, HLA-DQA1, HLA-DPB1, HLA-DRB1, HLA-DPA1;
(c) Coactivation molecules group: TNFRSF4, CD27, CD80, CD40LG, TNFRSF9, CD40, CD28, ICOSLG, CD83, TNFSF9, CD70, TNFSF4, ICOS, CD86;
(d) Effector cells group: ZAP70, GZMB, GZMK, IFNG, FASLG, EOMES, TBX21, GZMA, CD8A, GNLY, PRF1, CD8B;
(e) Natural killer cells group: NKG7, FGFBP2, CD244, KLRK1, KIR2DL4, CD226, KLRF1, GNLY, GZMB, KLRC2, NCR1, GZMH, IFNG, SH2D1B, NCR3, EOMES, CD160;
(f) T cells group: TRBC2, CD3E, CD3G, ITK, CD28, TRBC1, TRAT1, TBX21, CD5, TRAC, CD3D;
(g) T-helper cells type 1 group: IL21, TBX21, IL12RB2, CD40LG, IFNG, IL2, STAT4;
(h) T-helper cells type 2 group: IL13, CCR4, IL10, IL4, IL5;
(i) B cells group: CD22, TNFRSF13C, STAP1, CD79B, PAX5, CR2, TNFRSF13B, CD79A, TNFRSF17, FCRL5, MS4A1, CD19, BLK;
(j) Macrophages group: MRC1, SIGLEC1, MSR1, CD163, CSF1R, CD68, IL4I1, IL10;
(k) Macrophages type 1 group: CMKLR1, SOCS3, IRF5, NOS2, IL1B, IL12B, IL23A, TNF, IL12A;
(l) Antitumor cytokines group: CCL3, IL21, IFNB1, IFNA2, TNF, TNFSF10;
(m) Checkpoint inhibition group: PDCD1, BTLA, HAVCR2, CD274, VSIR, LAG3, TIGIT, PDCD1LG2, CTLA4;
(n) T-regulatory cells group: IKZF2, TNFRSF18, IL10, FOXP3, CCR8, IKZF4, CTLA4;
(o) Neutrophils group: CD177, FFAR2, PGLYRP1, CXCR1, MPO, CXCR2, ELANE, CTSG, PRTN3, FCGR3B;
(p) MDSC group: ARG1, IL6, CYBB, IL10, PTGS2, IDO1, IL4I1;
(q) Protumor cytokines group: TGFB2, MIF, IL6, TGFB3, TGFB1, IL22, IL10;
(r) Cancer associated fibroblasts (CAF) group: COL6A3, PDGFRB, COL6A1, MFAP5, COL5A1, FAP, PDGFRA, FGF2, ACTA2, COL6A2, FBLN1, CD248, COL1A1, MMP2, COL1A2, MMP3, LUM, CXCL12, LRP1;
(s) Matrix group: LAMC2, TNC, COL11A1, VTN, LAMB3, COL1A1, FN1, LAMA3, LGALS9, COL1A2, COL4A1, COL5A1, ELN, LGALS7, COL3A1;
(t) Matrix remodeling group: ADAMTS4, ADAMTS5, CA9, LOX, MMP1, MMP11, MMP12, MMP2, MMP3, MMP7, MMP9, PLOD2;
(u) Angiogenesis group: VEGFC, VEGFA, PDGFC, KDR, CDH5, VEGFB, PGF, TEK, ANGPT2, CXCR2, FLT1, CXCL8, VWF, ANGPT1, CXCL5;
(v) Endothelium group: KDR, CDH5, NOS3, VCAM1, VWF, FLT1, MMRN1, CLEC14A, ENG, MMRN2;
(w) Proliferation_rate group: CCND1, CCNB1, CETN3, CDK2, E2F1, AURKA, BUB1, AURKB, PLK1, MCM6, ESCO2, MYBL2, MKI67, MCM2, CCNE1; and
(x) Epithelial to mesenchymal transition group: CDH2, ZEB1, ZEB2, TWIST1, SNAI1, SNAI2, TWIST2.

In some embodiments, determining the gene group scores comprises determining gene group scores for each of the following gene groups using a single-sample GSEA (ssGSEA) technique from RNA expression levels for all the genes in each of the following gene groups:
(a) MHC type I group: HLA-C, TAPBP, HLA-B, B2M, TAP2, HLA-A, TAP1, NLRC5;
(b) MHC type II group: HLA-DQB1, HLA-DMA, HLA-DMB, HLA-DRA, CIITA, HLA-DQA1, HLA-DPB1, HLA-DRB1, HLA-DPA1;
(c) Coactivation molecules group: TNFRSF4, CD27, CD80, CD40LG, TNFRSF9, CD40, CD28, ICOSLG, CD83, TNFSF9, CD70, TNFSF4, ICOS, CD86;
(d) Effector cells group: ZAP70, GZMB, GZMK, IFNG, FASLG, EOMES, TBX21, GZMA, CD8A, GNLY, PRF1, CD8B;
(e) Natural killer cells group: NKG7, FGFBP2, CD244, KLRK1, KIR2DL4, CD226, KLRF1, GNLY, GZMB, KLRC2, NCR1, GZMH, IFNG, SH2D1B, NCR3, EOMES, CD160;
(f) T cells group: TRBC2, CD3E, CD3G, ITK, CD28, TRBC1, TRAT1, TBX21, CD5, TRAC, CD3D;
(g) T-helper cells type 1 group: IL21, TBX21, IL12RB2, CD40LG, IFNG, IL2, STAT4;
(h) T-helper cells type 2 group: IL13, CCR4, IL10, IL4, IL5;
(i) B cells group: CD22, TNFRSF13C, STAP1, CD79B, PAX5, CR2, TNFRSF13B, CD79A, TNFRSF17, FCRL5, MS4A1, CD19, BLK;
(j) Macrophages group: MRC1, SIGLEC1, MSR1, CD163, CSF1R, CD68, IL4I1, IL10;
(k) Macrophages type 1 group: CMKLR1, SOCS3, IRF5, NOS2, IL1B, IL12B, IL23A, TNF, IL12A;
(l) Antitumor cytokines group: CCL3, IL21, IFNB1, IFNA2, TNF, TNFSF10;
(m) Checkpoint inhibition group: PDCD1, BTLA, HAVCR2, CD274, VSIR, LAG3, TIGIT, PDCD1LG2, CTLA4;
(n) T-regulatory cells group: IKZF2, TNFRSF18, IL10, FOXP3, CCR8, IKZF4, CTLA4;
(o) Neutrophils group: CD177, FFAR2, PGLYRP1, CXCR1, MPO, CXCR2, ELANE, CTSG, PRTN3, FCGR3B;
(p) MDSC group: ARG1, IL6, CYBB, IL10, PTGS2, IDO1, IL4I1;
(q) Protumor cytokines group: TGFB2, MIF, IL6, TGFB3, TGFB1, IL22, IL10;
(r) Cancer associated fibroblasts (CAF) group: COL6A3, PDGFRB, COL6A1, MFAP5, COL5A1, FAP, PDGFRA, FGF2, ACTA2, COL6A2, FBLN1, CD248, COL1A1, MMP2, COL1A2, MMP3, LUM, CXCL12, LRP1;
(s) Matrix group: LAMC2, TNC, COL11A1, VTN, LAMB3, COL1A1, FN1, LAMA3, LGALS9, COL1A2, COL4A1, COL5A1, ELN, LGALS7, COL3A1;
(t) Matrix remodeling group: ADAMTS4, ADAMTS5, CA9, LOX, MMP1, MMP11, MMP12, MMP2, MMP3, MMP7, MMP9, PLOD2;
(u) Angiogenesis group: VEGFC, VEGFA, PDGFC, KDR, CDH5, VEGFB, PGF, TEK, ANGPT2, CXCR2, FLT1, CXCL8, VWF, ANGPT1, CXCL5;
(v) Endothelium group: KDR, CDH5, NOS3, VCAM1, VWF, FLT1, MMRN1, CLEC14A, ENG, MMRN2;
(w) Proliferation_rate group: CCND1, CCNB1, CETN3, CDK2, E2F1, AURKA, BUB1, AURKB, PLK1, MCM6, ESCO2, MYBL2, MKI67, MCM2, CCNE1;
(x) Epithelial to mesenchymal transition group: CDH2, ZEB1, ZEB2, TWIST1, SNAI1, SNAI2, TWIST2;
(y) Luminal differentiation group: PWRN1, PWRN3, GSTM5, GSTM4, GSTM2, ZNF321P, ZNF320, ZNF66, ZNF737, KRT20, UPK1B, FOXA1, ACER2, SEMA5A, PPARG, GATA3, SNX31, UPK2, UPK1A;
(z) Basal differentiation group: TM4SF19, SERPINB13, SERPINB3, SERPINB4, SPRR2F, SPRR2E, SPRR2A, SPRR2D, KRT17, KRT16, KRT14, DSG3, KRT5, KRT6C, KRT6A, KRT6B;
(aa) Neuroendocrine differentiation group: PLEKHG4B, GNG4, PEG10, SOX2, TUBB2B, CHGB, SYP, ENO2, SV2A, MSI1, RND2, APLP1; and
(bb) FGFR3 co-expressed group: FGFR3, TP63, IRS1, WNT7B, CAPNS2, ZNF385A, SMAD3, SLC2A9, DUOXA1, SYTL1, SEMA4B, CLCA4, PLCH2, SSH3, PTPN13, TMPRSS4.

In some embodiments, generating a UC TME signature further comprises normalizing the gene group scores, wherein the normalizing comprises performing a median scaling calculation on the gene group scores.

In some embodiments, the plurality of UC TME types is associated with a respective plurality of UC TME signature clusters, wherein identifying, using the UC TME signature and from among a plurality of UC TME types, the UC TME type for the subject comprises associating the UC TME signature of the subject with a particular one of the plurality of UC TME signature clusters; and identifying the UC TME type for the subject as the UC TME type corresponding to the particular one of the plurality of UC TME signature clusters to which the UC TME signature of the subject is associated.

In some embodiments, methods described by the disclosure further comprise generating a plurality of UC TME signature clusters, the generating comprising obtaining multiple sets of RNA expression data by sequencing biological samples from multiple respective subjects, each of the multiple sets of expression data indicating RNA expression levels for genes in a plurality of gene groups listed in Table 1; generating multiple UC TME signatures from the multiple sets of RNA expression data, each of the multiple UC TME signatures comprising gene group scores for respective gene groups in the plurality of gene groups, the generating comprising, for each particular one of the multiple UC TME signatures determining the UC TME signature by determining the gene group scores using the RNA expression levels in the particular set of RNA expression data for which the particular one UC TME signature is being generated; and clustering the multiple UC signatures to obtain the plurality of UC TME signature clusters.

In some embodiments, clustering is performed using a clustering algorithm. In some embodiments, the clustering algorithm is a dense clustering algorithm, spectral clustering algorithm, k-means clustering algorithm, hierarchical clustering algorithm, and/or an agglomerative clustering algorithm. In some embodiments, the clustering comprises using a Consensus Clustering or Louvain density clustering technique.

In some embodiments, methods described by the disclosure further comprise updating the plurality of UC TME signature clusters using the UC TME signature of the subject, wherein the UC TME signature of the subject is one of a threshold number UC TME signatures for a threshold number of subjects, wherein when the threshold number of UC TME signatures is generated the UC TME signature clusters are updated. In some embodiments, a threshold number of UC TME signatures is at least 50, at least 75, at least 100, at least 200, at least 500, at least 1000, or at least 5000 UC TME signatures.

In some embodiments, updating is performed using a clustering algorithm. In some embodiments, the clustering algorithm is a dense clustering algorithm, spectral clustering algorithm, k-means clustering algorithm, hierarchical clustering algorithm, and/or an agglomerative clustering algorithm. In some embodiments, the clustering comprises using a Consensus Clustering or Louvain density clustering technique.

In some embodiments, methods described by the disclosure further comprise determining an UC TME type of a second subject, wherein the UC TME type of the second subject is identified using the updated UC TME signature clusters, wherein the identifying comprises: determining an UC TME signature of the second subject from RNA expression data obtained by sequencing a biological sample obtained from the second subject; associating the UC TME signature of the second subject with a particular one of the plurality of the updated UC TME signature clusters; and identifying the UC TME type for the second subject as the UC TME type corresponding to the particular one of the plurality of updated UC TME signature clusters to which the UC TME signature of the second subject is associated.

In some embodiments, the plurality of a plurality of UC TME types comprises: Immune Desert (D) type, Immune Enriched (IE) type, Fibrotic (F) type, Immune Enriched -Fibrotic (IE/F) type, Immune Desert type, FGFR-altered (D/FGFR) type, Fibrotic - Basal (Bas) type, and Neuroendocrine-like (NE) type.

In some embodiments, methods described by the disclosure further comprise identifying the subject as a candidate for treatment with an anti-FGFR agent when the subject is identified as having Desert, FGFR-altered type UC TME.

In some embodiments, methods described by the disclosure further comprise identifying the subject as a candidate for treatment with an ERBB2-targeting therapy or PARP inhibitor when the subject is identified as having Desert type UC TME.

In some embodiments, methods described by the disclosure further comprise identifying the subject as a candidate for treatment with an immune checkpoint inhibitor (ICI) when the subject is identified as having Immune Enriched type UC TME.

In some embodiments, methods described by the disclosure further comprise identifying the subject as a candidate for treatment with a TGFb inhibitor or PARP inhibitor when the subject is identified as having Fibrotic type UC TME.

In some embodiments, methods described by the disclosure further comprise identifying the subject as a candidate for treatment with an immune checkpoint inhibitor (ICI) when the subject is identified as having Immune Enriched, Fibrotic type UC TME.

In some embodiments, methods described by the disclosure further comprise the subject as having a poor prognosis when the subject has Fibrotic, Basal type UC TME.

In some embodiments, methods described by the disclosure further comprise identifying the subject as a candidate for treatment with an immune checkpoint inhibitor (ICI) when the subject is identified as having Neuroendocrine-like type UC TME. In some embodiments, an ICI is atezolizumab.

In some embodiments, methods described by the disclosure further comprise administering a therapeutic agent to the subject based upon the identification of the subject’s UC TME type. In some embodiments, a therapeutic agent comprises an immune checkpoint inhibitor (ICI), TGFb inhibitor, ERBB2-targeting therapy, or a PARP inhibitor.

In some aspects, the disclosure provides a method for determining a urothelial cancer (UC) mutational subtype of a subject having, suspected of having, or at risk of having a urothelial cancer, the method comprising using at least one computer hardware processor to perform obtaining RNA expression data for the subject, the RNA expression data indicating RNA expression levels for genes of the subject; generating a UC mutational subtype signature for the subject using the RNA expression data, the generating comprising analyzing the RNA expression data to identify the presence or absence of one or more mutations in the one or more of the following genes: ERCC2, FGFR3, PIK3CA, ARID1A, ATM, CDKN1A, CREBBP, FAT1, FBXW7, KDM6A, RB1, RHOB, TP53, TSC1, HRAS, KRAS, and NRAS; and identifying, using the UC mutational subtype signature and from among a plurality of UC mutational subtypes, a UC mutational subtype for the subject.

In some embodiments, the plurality of UC mutational subtypes is associated with a respective plurality of UC mutational subtype clusters, wherein identifying, using the UC mutational subtype signature and from among a plurality of UC mutational subtypes, the UC mutational subtype for the subject comprises associating the UC mutational subtype signature of the subject with a particular one of the plurality of UC mutational subtype clusters; and, identifying the UC mutational subtype for the subject as the UC mutational subtype corresponding to the particular one of the plurality of UC mutational subtype clusters to which the UC mutational subtype signature of the subject is associated.

In some embodiments, the method further comprises generating the plurality of UC mutational subtype clusters, the generating comprising obtaining multiple sets of RNA expression data by sequencing biological samples from multiple respective subjects, each of the multiple sets of expression data indicating RNA expression levels for genes in the subjects; generating multiple UC mutational subtype signatures from the multiple sets of RNA expression data, the generating comprising, for each particular one of the multiple UC mutational subtype signatures analyzing the particular set of RNA expression data for which the particular one UC mutational subtype signature is being generated to identify the presence or absence of one or more mutations in the one or more of the following genes: ERCC2, FGFR3, PIK3CA, ARID1A, ATM, CDKN1A, CREBBP, FAT1, FBXW7, KDM6A, RB1, RHOB, TP53, TSC1, HRAS, KRAS, and NRAS; and clustering the multiple UC mutational subtype signatures to obtain the plurality of UC mutational subtype clusters.

In some embodiments, the clustering comprises using a non-negative matrix factorization (NMF) approach. In some embodiments, the NMF approach comprises a Hierarchical Dirichlet Process and/or CoGAPS.

In some embodiments, the plurality of a plurality of UC mutational subtype clusters comprises: TP53-altered type, KDM6A-altered type, FGFR3-altered type, ARID1A-altered type, and Hypermutated (“HM”) type.

In some embodiments, methods described by the disclosure further comprise identifying the subject as a candidate for treatment with an immune checkpoint inhibitor (ICI) when the subject is identified as having TP53-altered type, ARID1A-altered type, or Hypermutated (“HM”) type UC mutational subtype.

In some embodiments, methods described by the disclosure further comprise identifying the subject as a candidate for treatment with an anti-FGFR agent when the subject is identified as having FGFR3-altered type UC mutational subtype.

In some embodiments, methods described by the disclosure further comprise identifying the subject as a candidate for treatment with cisplatin when the subject is identified as having ARID1A-altered type UC mutational subtype.

In some embodiments, methods described by the disclosure further comprise administering a therapeutic agent to the subject based upon the identification of the subject’s UC mutational subtype.

In some aspects the disclosure provides a system, comprising at least one computer hardware processor; and at least one computer-readable storage medium storing processor-executable instructions that, when executed by the at least one computer hardware processor, cause the at least one computer hardware processor to perform a method for determining a urothelial cancer (UC) tumor microenvironment (TME) type of a subject having, suspected of having, or at risk of having a urothelial cancer, the method comprising obtaining RNA expression data for the subject, the RNA expression data indicating RNA expression levels for at least some genes in each group of at least some of a plurality of gene groups listed in Table 1; generating a UC TME signature for the subject using the RNA expression data, the UC TME signature comprising gene group scores for respective gene groups in the at least some of the plurality of gene groups, the generating comprising: determining the gene group scores using the RNA expression levels; and identifying, using the UC TME signature and from among a plurality of UC TME types, a UC TME type for the subject.

In some aspects, the disclosure provides at least one computer-readable storage medium storing processor-executable instructions that, when executed by at least one computer hardware processor, cause the at least one computer hardware processor to perform a method for determining a urothelial cancer (UC) tumor microenvironment (TME) type of a subject having, suspected of having, or at risk of having a urothelial cancer, the method comprising obtaining RNA expression data for the subject, the RNA expression data indicating RNA expression levels for at least some genes in each group of at least some of a plurality of gene groups listed in Table 1; generating a UC TME signature for the subject using the RNA expression data, the UC TME signature comprising gene group scores for respective gene groups in the at least some of the plurality of gene groups, the generating comprising: determining the gene group scores using the RNA expression levels; and identifying, using the UC TME signature and from among a plurality of UC TME types, a UC TME type for the subject.

In some aspects, the disclosure provides a system, comprising at least one computer hardware processor; and at least one computer-readable storage medium storing processor-executable instructions that, when executed by the at least one computer hardware processor, cause the at least one computer hardware processor to perform a method for determining a urothelial cancer (UC) mutational subtype of a subject having, suspected of having, or at risk of having a urothelial cancer, the method comprising using at least one computer hardware processor to perform obtaining RNA expression data for the subject, the RNA expression data indicating RNA expression levels for genes of the subject; generating a UC mutational subtype signature for the subject using the RNA expression data, the generating comprising analyzing the RNA expression data to identify the presence or absence of one or more mutations in the one or more of the following genes: ERCC2, FGFR3, PIK3CA, ARID1A, ATM, CDKN1A, CREBBP, FAT1, FBXW7, KDM6A, RB1, RHOB, TP53, TSC1, HRAS, KRAS, and NRAS; and, identifying, using the UC mutational subtype signature and from among a plurality of UC mutational subtypes, a UC mutational subtype for the subject.

In some aspects, the disclosure provides at least one computer-readable storage medium storing processor-executable instructions that, when executed by at least one computer hardware processor, cause the at least one computer hardware processor to perform a method for determining a urothelial cancer (UC) mutational subtype of a subject having, suspected of having, or at risk of having a urothelial cancer, the method comprising using at least one computer hardware processor to perform obtaining RNA expression data for the subject, the RNA expression data indicating RNA expression levels for genes of the subject; generating a UC mutational subtype signature for the subject using the RNA expression data, the generating comprising analyzing the RNA expression data to identify the presence or absence of one or more mutations in the one or more of the following genes: ERCC2, FGFR3, PIK3CA, ARID1A, ATM, CDKN1A, CREBBP, FAT1, FBXW7, KDM6A, RB1, RHOB, TP53, TSC1, HRAS, KRAS, and NRAS; and, identifying, using the UC mutational subtype signature and from among a plurality of UC mutational subtypes, a UC mutational subtype for the subject.

### BRIEF DESCRIPTION OF DRAWINGS

FIG. 1 provides an example of a processes for identifying the urothelial cancer (UC) TME type of a subject, according to some aspects of the invention. In some embodiments, the process includes obtaining a biopsy sample of a subject, extracting nucleic acids from the sample, sequencing the nucleic acids, and analyzing the nucleic acid sequences to identify a UC TME type for the subject based on the gene expression data.

FIG. 2 is a diagram depicting a flowchart of an illustrative process for processing sequencing data to obtain RNA expression data, according to some embodiments of the technology as described herein.

FIG. 3 is a diagram depicting an illustrative technique for determining gene group scores, according to some embodiments of the technology as described herein.

FIG. 4 is a diagram depicting an illustrative technique for identifying a urothelial cancer (UC) tumor microenvironment (TME) type using a UC TME signature, according to some embodiments of the technology as described herein.

FIG. 5 provides an example of a processes for identifying the urothelial cancer (UC) mutational subtype of a subject, according to some aspects of the invention. In some embodiments, the process includes obtaining a biopsy sample of a subject, extracting nucleic acids from the sample, sequencing the nucleic acids, and analyzing the nucleic acid sequences to identify a UC mutational subtype for the subject based on the gene expression data.

FIGS. 6A-6B show a representative heatmap of urothelial cancer (UC) samples classified into seven distinct UC TME types (Desert (D), Immune Enriched (IE), Fibrotic (F), Immune Enriched Fibrotic (IE/F), Desert FGFR-altered (D/FGFR), Basal (Bas; also referred to as “Fibrotic Basal”), and Neuroendocrine-like (NE)) based on unsupervised dense clustering of 28 gene expression signatures, according to some aspects of the invention. Each column represents one sample. All signatures were grouped into 4 categories (panel on the right): Angiogenesis and Fibroblasts, Pro and Anti-tumor immune infiltrate, and Tumor biology. FIGS. 6C-6D show a schematic depicting features of the seven distinct UC TME types (D, IE, F, IE/F, D/FGFR, Bas, NE), including differentiation pathway, TME composition, malignant cell percentage, malignant cell features, molecular alterations, and potential treatment options.

FIGS. 7A-7N show transcriptomic characterization of UC TME types: Desert, FGFR-altered (7A, 7B), Desert (7C, 7D), Immune Enriched (7E, 7F), Fibrotic (7G, 7H), Immune Enriched, Fibrotic (7I, 7J), Basal (also referred to as Fibrotic, Basal) (7K, 7L), Neuroendocrine-like (7M, 7N). FIGS. 7B, 7D, 7F, 7H, 7J, 7L and 7N depict visual reconstructions of the TME composition for each TME type. The Wilcoxon Rank Sum test was used to assess statistical significance, *** means p<0.001.

FIG. 8 shows a comparison of UC TME signatures three groups of UC TME types. The Luminal group includes Desert - FGFR-altered (D/FGFR), Desert (D), Immune Enriched (IE), and Fibrotic (F) TME types; the Basal group includes Immune Enriched - Fibrotic (IE/F), -and Basal (Bas; also referred to as “Fibrotic Basal”); and the Neuroendocrine group consists of the Neuroendocrine-like (NE) type. The Wilcoxon Rank Sum test was used to assess statistical significance, *** means p <0.001.

FIG. 9 shows a representative oncoplot. Each UC TME type is associated with specific mutations and copy number alterations (CNA). The Chi-square test and Benjamini-Hochberg correction were used to assess significance. * means p-adjusted < 0.05.

FIG. 10 shows histopathological patterns associated with UC TME types. Shown left to right and top to bottom are representative data for: invasiveness; histology (e.g., papillary vs. non-papillary); tumor stage (e.g., T0, T1, T2, T3, T4); Grade (e.g., low vs. high); Distant metastasis (M0, M1); lymph node (LN) metastasis (N0, N1, N2, N3); Luminal differentiation; and Basal differentiation.

FIG. 11 shows overall survival (OS) rate and cisplatin-based response across seven datasets for UC TME types (top), Anti-PD-L1 second-line therapy (bottom left) and response rate to anti-PD-L1 therapy (bottom right).

FIG. 12 shows overall survival (OS) rate for cisplatin-based treatment across UC TME types in the TCGA BLCA dataset (left) and the GSE13507 dataset (right).

FIG. 13 shows representative data indicating UC TME types (e.g., IE/F and Bas) better predict overall survival rate and response rate under atezolizumab therapy (right), an anti-PDL1 agent, than previously described urothelial cancer subtypes (BalSq) (left).

FIG. 14 shows representative data indicating overall survival (OS) rate as measured using cisplatin-based therapy across previously described classical molecular functional portraits (MFP) (top left), cisplatin-based therapy across UC TME types (top right), anti-PDL1 therapy across previously described classical MFP (bottom left), and anti-PDL1 therapy across novel UC TME types (bottom right).

FIG. 15 shows a representative oncoplot. Each UC Mutational Subtype (e.g., TP53-altered, FGFR3-altered, ARID1A-altered, KDM6A-altered; Hypermutated) is associated with specific mutations.

FIG. 16 shows representative data for overall survival (OS) rate and cisplatin-based therapy response and anti-PD-L1 second line therapy response across UC Mutational Subtypes.

FIG. 17 depicts an illustrative implementation of a computer system that may be used in connection with some embodiments of the technology described herein.

### DETAILED DESCRIPTION

Aspects of the disclosure relate to methods systems, and computer-readable storage media, which are useful for characterizing subjects having bladder cancers or urothelial cancers. The disclosure is based, in part, on methods for identifying the tumor microenvironment (TME) of a subject having urothelial cancer (e.g., urothelial carcinoma of the urinary bladder) by using gene expression data obtained from the subject to produce a urothelial cancer (UC) signature that, when processed by methods disclosed herein, allows for assignment of a UC type to the subject. The inventors have surprisingly discovered that using methods described herein to characterize UC patients resulted in the identification of two previously undescribed UC TME types, which allow for more accurate patient stratification and prognosis relative to previously described UC typing techniques. In some embodiments, UC TME types described herein may be used to identify one or more therapeutic agents that can be administered to the subject.

Bladder cancer is a group of solid tumor cancers that originate in bladder tissue and affect over 80,000 people each year. There are three histological types of bladder cancer- urothelial carcinoma (also referred to as urothelial cancer or transitional cell cancer), squamous cell carcinoma, and adenocarcinoma). Urothelial cancer (UC) is the most common histological type of bladder cancer. Typically, UC has a high rates of recurrence and disease progression, and is often resistant to standard therapeutic regimens. Response of UC patients to immunotherapy with immune checkpoint inhibitors (ICI) has been observed to be approximately 15-25%.

Bladder cancer may be also sub-classified according to a number of techniques. Classification of a subject’s bladder cancer type is an important process that may provide insight into tumor biology and the subject’s prognosis. Tumor classification may also guide a physician’s decisions on therapeutic and surgical interventions for a patient. Molecular characterization of UC has been described. For example, Kamoun et al. (European Urology, 77(4), 2020, 420-433; doi.org/10.1016/j.eururo.2019.09.006) describe six molecular subtypes of muscle-invasive bladder cancer: luminal papillary, luminal non-specified, luminal unstable, stroma-rich, basal/squamous, and neuroendocrine-like. However, molecular classification of UC into six intrinsic molecular types may not have high enough resolution to account for UC intra-tumoral heterogeneity, particularly within the basal/squamous subtype, and provide therapeutic recommendations for UC patients, for example as described by Fong et al. (Update on bladder cancer molecular subtypes. Transl Androl Urol 2020;9(6):2881-2889. doi: 10.21037/tau-2019-mibc-12).

Aspects of the disclosure relate to statistical techniques for analyzing expression data (e.g., RNA expression data), which was obtained from a biological sample obtained from a subject that has urothelial cancer, is suspected of having urothelial cancer, or is at risk of developing urothelial cancer, in order to generate a gene expression signature for the subject (termed a “TME signature” herein) and use this signature to identify a particular TME type that the subject may have.

The inventors have recognized that certain previously-described molecular subtypes of urothelial cancer (e.g., basal/squamous UC as described by Kamoun) may be further separated into phenotypically distinct TME types within each UC subtype. For example, the inventors have recognized that the basal/squamous UC subtype may be further divided into two phenotypically distinct types based upon the tumor microenvironment (TME) of the cancer, “Fibrotic, Basal” (also referred to as “Basal”) and “Immune Enriched, Fibrotic” (IE/F). The tumor microenvironment of UC may also be further characterized into five other TME types (in addition to the Basal and IE/F TME types described above): Immune Desert (D) type, Immune Enriched (IE) type, Fibrotic (F) type, Immune Desert type, FGRF-altered (D/FGFR) type, and Neuroendocrine-like (NE) type. Together, these seven TME types of UC reflect not only the TME of the cancer but also genomic drivers and malignant cell features that underly the biological processes occurring in the UC patient. As described further in the Examples, each UC TME type was identified using a combination of gene group expression scores to produce a UC TME signature that characterizes patients having UC more accurately than previously developed methods. In some embodiments, such TME types are useful for identifying the prognosis and/or likelihood that a subject will respond to particular therapeutic interventions (e.g., immunotherapy agents, anti-FGFR3 agents, platinum-based therapies (e.g., cisplatin, etc.), etc.).

The use of TME signatures comprising the combinations of gene group scores described by the disclosure represents an improvement over previously described molecular characterization of UC because the specific groups of genes used to produce the TME signatures described herein better reflect the molecular tumor microenvironments (TME) of urothelial cancer because these gene groups are associated with the underlying biological pathways controlling tumor behavior and the host tumor microenvironment. These focused combinations of gene groups (e.g., gene groups consisting of some or all of the gene group genes listed in Table 1) are unconventional, and differ from previously described molecular signatures, which do not account for the high levels of genotypic and phenotypic heterogeneity within each broad molecular subtype of UC.

The TME typing methods described herein have several utilities. For example, identifying a subject’s TME type using methods described herein may allow for the subject to be diagnosed as having (or being at a high risk of developing) an aggressive form of UC (e.g., Basal UC TME type) at a timepoint that is not possible with previously described UC characterization methods. Earlier detection of aggressive UC types, enabled by the TME signatures described herein, improve the patient diagnostic technology by enabling earlier chemotherapeutic or radiotherapeutic intervention for patients than currently possible for patients tested for UC using other methods (e.g., histological analysis).

As described herein, the inventors have also determined that subjects identified by methods described herein as having certain UC TME types (e.g., UC TME type IE, IE/F, or Ne-like) are characterized has having an increased likelihood of responding to immunotherapeutic agents, for example immune checkpoint inhibitors (ICI). Conversely, the inventors have determined that subjects having other TME types (e.g., UC TME type D, D/FGFR, Bas) are characterized has having an increased likelihood of responding to non-ICI therapeutic agents, such as PARP inhibitors, anti-FGFR3 agents, ERBB2 inhibitors, cisplatin, etc. Thus, the techniques developed by the inventors and described herein improve patient treatment and associated outcomes by increasing patient comfort, and avoiding toxic side effects of chemotherapy that is not expected to be effective for the subject.

Aspects of the disclosure relate to statistical techniques for analyzing expression data (e.g., RNA expression data), which was obtained from a biological sample obtained from a subject that has urothelial cancer, is suspected of having urothelial cancer, or is at risk of developing urothelial cancer, in order to generate a gene expression signature for the subject (termed a “mutational subtype signature” herein) and use this signature to identify a particular UC mutational type that the subject may have.

The inventors have recognized that UC patients may be classified into five different mutational subtypes based on the character and number of genetic alterations (e.g., mutations, copy number alterations (CNA), etc.) present in the cells of the subject’s tumor microenvironment (TME). The five mutational subtypes of UC identified by the inventors are: TP53-altered, KDM6A-altered, FGFR3-altered, and ARID1A-altered, and Hypermutated (“HM”). The use of UC mutational subtype signatures comprising the combinations of gene group scores described by the disclosure represents an improvement over previously described molecular characterization of UC because the specific groups of genes used to produce the mutational subtype signatures described herein better reflect the influence of genetic drivers of UC in the TME and the effects of those drivers on therapeutic response.

As described herein, the inventors have determined that subjects identified by methods described herein as having certain UC mutational subtypes (e.g., UC mutational subtype TP53, ARID1A, or HM) are characterized has having an increased likelihood of responding to immunotherapeutic agents, for example immune checkpoint inhibitors (ICI). Conversely, the inventors have determined that subjects having other UC mutational subtypes (e.g., UC mutational subtype KDM6A or FGFR3) are characterized has having an increased likelihood of responding to non-ICI therapeutic agents, such as PARP inhibitors, anti-FGFR3 agents, ERBB2 inhibitors, cisplatin, etc. Thus, the techniques developed by the inventors and described herein improve patient treatment and associated outcomes by increasing patient comfort, and avoiding toxic side effects of chemotherapy that is not expected to be effective for the subject.

### Urothelial Cancers

Aspects of the disclosure relate to identifying the tumor microenvironment (TME) type (also referred to as the urothelial cancer (UC) type) of a subject. As used herein, the term “subject” means any mammal, including mice, rabbits, and humans. In one embodiment, the subject is a human or non-human primate. The terms “individual” or “subject” may be used interchangeably with “patient.” In some embodiments, the biological sample may be any sample from a subject known or suspected of having cancerous cells or pre-cancerous cells.

In some embodiments, a subject has, is suspected of having, or at risk of developing cancer. As used herein, “cancer” refers to any malignant and/or invasive growth or tumor caused by abnormal cell growth in a subject, including solid tumors, blood cancer, bone marrow or lymphoid cancer, etc. A subject “having cancer” exhibits one or more signs or symptoms of cancer, for example the presence of cancerous cells (e.g., tumor cells). In some embodiments, a subject having cancer has been diagnosed as having cancer by a clinician (e.g., physician) and/or has received a positive result of a laboratory test that indicates the subject as having cancer. A subject “suspected of having cancer” exhibits one or more signs or symptoms of cancer (e.g., presence of a tumor or tumor cells, fever, swelling, bleeding, etc.) but has not been diagnosed by a clinician as having cancer. A subject “at risk of having cancer” may or may not exhibit one or more signs or symptoms of cancer but may comprise one or more genetic mutations that increases the risk that the subject will develop cancer (e.g., relative to a normal healthy subject not having such mutations).

In some embodiments, the cancer is a bladder cancer. Examples of bladder cancers include but are not limited to transitional cell (urothelial) bladder cancer (e.g., plasmacytoid, nested, micropapillary, lipoid cell, sarcomatoid, microcystic, lymphoepithelioma-like, inverted papilloma-like, clear cell, etc.), squamous cell bladder cancer, adenocarcinoma of the bladder, sarcoma of the bladder, and small cell cancer of the bladder.

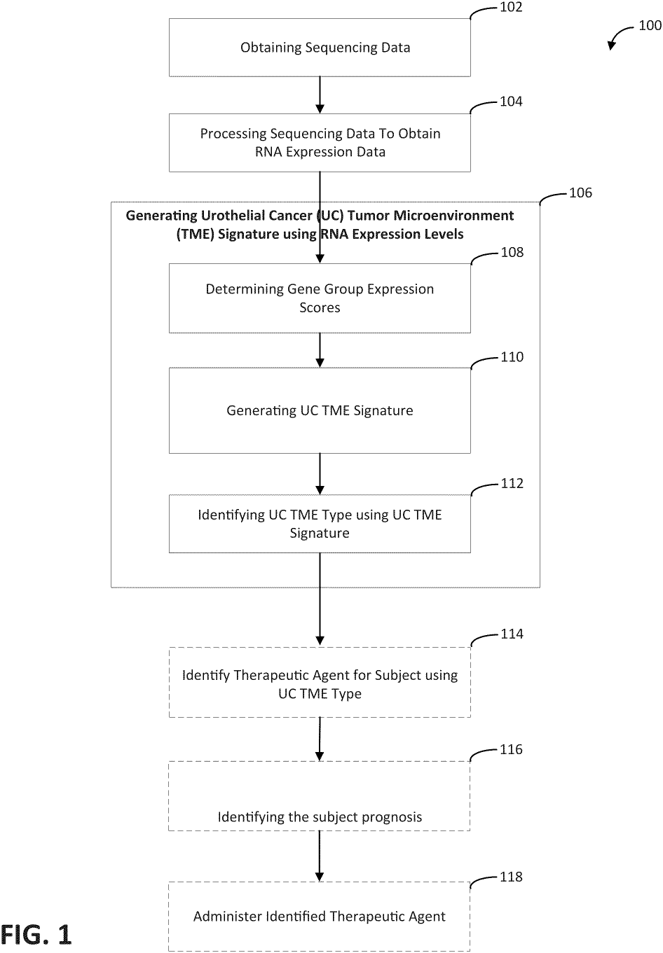

FIG. 1 is a flowchart of an illustrative process 100 for determining a UC TME signature for a subject, using the determined UC TME signature to identify the UC TME type for the subject, and using the UC TME type of the subject to identify whether or not the subject is likely to respond to a therapy, e.g., an immunotherapy, anti-FGFR3 agent, platinum-based agent, etc.

Various (e.g., some or all) acts of process 100 may be implemented using any suitable computing device(s). For example, in some embodiments, one or more acts of the illustrative process 100 may be implemented in a clinical or laboratory setting. For example, one or more acts of the process 100 may be implemented on a computing device that is located within the clinical or laboratory setting. In some embodiments, the computing device may directly obtain RNA expression data from a sequencing apparatus located within the clinical or laboratory setting. For example, a computing device included in the sequencing apparatus may directly obtain the RNA expression data from the sequencing apparatus. In some embodiments, the computing device may indirectly obtain RNA expression data from a sequencing apparatus that is located within or external to the clinical or laboratory setting. For example, a computing device that is located within the clinical or laboratory setting may obtain expression data via a communication network, such as Internet or any other suitable network, as aspects of the technology described herein are not limited to any particular communication network.

Additionally or alternatively, one or more acts of the illustrative process 100 may be implemented in a setting that is remote from a clinical or laboratory setting. For example, the one or more acts of process 100 may be implemented on a computing device that is located externally from a clinical or laboratory setting. In this case, the computing device may indirectly obtain RNA expression data that is generated using a sequencing apparatus located within or external to a clinical or laboratory setting. For example, the expression data may be provided to computing device via a communication network, such as Internet or any other suitable network.

It should be appreciated that, in some embodiments, not all acts of process 100, as illustrated in FIG. 1, may be implemented using one or more computing devices. For example, the act 118 of administering one or more therapeutic agents to the subject may be implemented manually (e.g., by a clinician).

Process 100 begins at act 102 where sequencing data for a subject is obtained. In some embodiments, the sequencing data may be obtained by sequencing a biological sample (e.g., bladder tissue biopsy and/or tumor tissue) obtained from the subject using any suitable sequencing technique. The sequencing data may include sequencing data of any suitable type, from any suitable source, and be in any suitable format. Examples of sequencing data, sources of sequencing data, and formats of sequencing data are described herein including in the section called “Obtaining RNA Expression Data.”

As one illustrative example, in some embodiments, the sequencing data may comprise bulk sequencing data. The bulk sequencing data may comprise at least 1 million reads, at least 5 million reads, at least 10 million reads, at least 20 million reads, at least 50 million reads, or at least 100 million reads. In some embodiments, the sequencing data comprises bulk RNA sequencing (RNA-seq) data, single cell RNA sequencing (scRNA-seq) data, or next generation sequencing (NGS) data. In some embodiments, the sequencing data comprises microarray data.

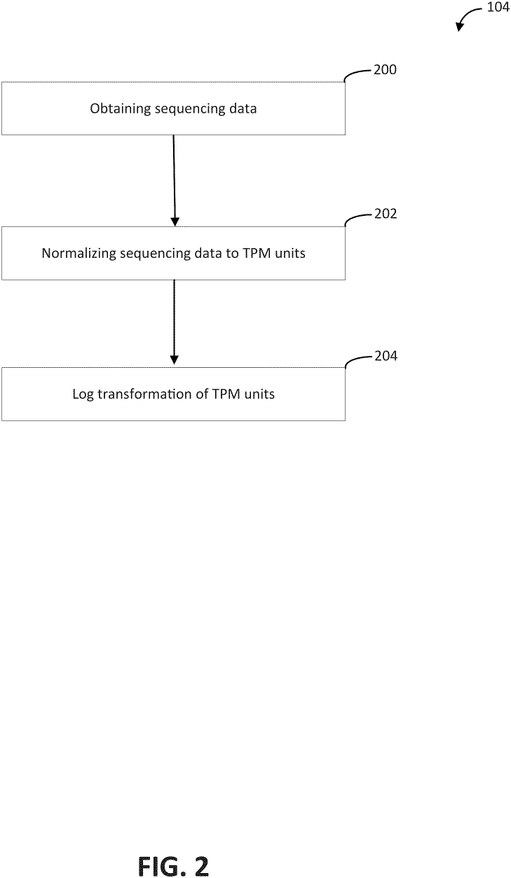

Next, process 100 proceeds to act 104, where the sequencing data obtained at act 102 is processed to obtain RNA expression data. This may be done in any suitable way and may involve normalizing bulk sequencing data to transcripts-per-million (TPM) units (or other units) and/or log transforming the RNA expression levels in TPM units. Converting the data to TPM units and normalization are described herein including with reference to FIG. 2.

Next, process 100 proceeds to act 106, where a urothelial cancer (UC) tumor microenvironment (TME) signature is generated for the subject using the RNA expression data generated at act 104 (e.g., from bulk-sequencing data, converted to TPM units and subsequently log-normalized, as described herein including with reference to FIG. 2).

As described herein, in some embodiments, a UC TME signature comprises two or more (e.g., 2, 3, 4, 5, 6, 7, 8, 9, 10, 11, 12, 13, 14, 15, 16, 17, 18, 19, 20, 21, 22, 23, 24, 25, 26, 27, 28, etc.) gene group scores. In some embodiments, the two or more gene group scores comprise gene group scores (which may also be referred to as gene group enrichment scores or gene group expression scores) for some or all of the gene groups shown in Table 1.

Accordingly, act 106 comprises: act 108 where the gene group scores are determined, act 110 where the UC TME signature is generated using the gene group determined at act 108, and act 112 where the UC TME type is determined by using the UC TME signature determined at act 110. In some embodiments, determining the gene group scores comprises determining, for each of multiple (e.g., some or all of the) gene groups listed in Table 1, a respective gene group score. In some embodiments, determining the gene group scores comprises determining respective gene group scores for 2, 3, 4, 5, 6, 7, 8, 9, 10, 11, 12, 13, 14, 15, 16, 17, 18, 19, 20, 21, 22, 23, 24, 25, 26, 27, or 28 gene groups (e.g., gene groups listed in Table 1). The gene group score for a particular gene group may be determined using RNA expression levels for at least some of the genes in the gene group (e.g., the RNA expression levels obtained at act 104). The RNA expression levels may be processed using a gene set enrichment analysis (GSEA) technique to determine the score for the particular gene group.

For example, in some embodiments, determining the UC TME signature comprises: determining gene group scores using the RNA expression levels for at least three genes from each of at least two of the gene groups, the gene groups including: MHC type I group: HLA-C, TAPBP, HLA-B, B2M, TAP2, HLA-A, TAP1, NLRC5; MHC type II group: HLA-DQB1, HLA-DMA, HLA-DMB, HLA-DRA, CIITA, HLA-DQA1, HLA-DPB1, HLA-DRB1, HLA-DPA1; Coactivation molecules group: TNFRSF4, CD27, CD80, CD40LG, TNFRSF9, CD40, CD28, ICOSLG, CD83, TNFSF9, CD70, TNFSF4, ICOS, CD86; Effector cells group: ZAP70, GZMB, GZMK, IFNG, FASLG, EOMES, TBX21, GZMA, CD8A, GNLY, PRF1, CD8B; Natural killer cells group: NKG7, FGFBP2, CD244, KLRK1, KIR2DL4, CD226, KLRF1, GNLY, GZMB, KLRC2, NCR1, GZMH, IFNG, SH2D1B, NCR3, EOMES, CD160; T cells group: TRBC2, CD3E, CD3G, ITK, CD28, TRBC1, TRAT1, TBX21, CD5, TRAC, CD3D; T-helper cells type 1 group: IL21, TBX21, IL12RB2, CD40LG, IFNG, IL2, STAT4; T-helper cells type 2 group: IL13, CCR4, IL10, IL4, IL5; B cells group: CD22, TNFRSF13C, STAP1, CD79B, PAX5, CR2, TNFRSF13B, CD79A, TNFRSF17, FCRL5, MS4A1, CD19, BLK; Macrophages group: MRC1, SIGLEC1, MSR1, CD163, CSF1R, CD68, IL4I1, IL10; Macrophages type 1 group: CMKLR1, SOCS3, IRF5, NOS2, IL1B, IL12B, IL23A, TNF, IL12A; Antitumor cytokines group: CCL3, IL21, IFNB1, IFNA2, TNF, TNFSF10; Checkpoint inhibition group: PDCD1, BTLA, HAVCR2, CD274, VSIR, LAG3, TIGIT, PDCD1LG2, CTLA4; T-regulatory cells group: IKZF2, TNFRSF18, IL10, FOXP3, CCR8, IKZF4, CTLA4; Neutrophils group: CD177, FFAR2, PGLYRP1, CXCR1, MPO, CXCR2, ELANE, CTSG, PRTN3, FCGR3B; MDSC group: ARG1, IL6, CYBB, IL10, PTGS2, IDO1, IL4I1; Protumor cytokines group: TGFB2, MIF, IL6, TGFB3, TGFB1, IL22, IL10; Cancer associated fibroblasts (CAF) group: COL6A3, PDGFRB, COL6A1, MFAP5, COL5A1, FAP, PDGFRA, FGF2, ACTA2, COL6A2, FBLN1, CD248, COL1A1, MMP2, COL1A2, MMP3, LUM, CXCL12, LRP1; Matrix group: LAMC2, TNC, COL11A1, VTN, LAMB3, COL1A1, FN1, LAMA3, LGALS9, COL1A2, COL4A1, COL5A1, ELN, LGALS7, COL3A1; Matrix remodeling group: ADAMTS4, ADAMTS5, CA9, LOX, MMP1, MMP11, MMP12, MMP2, MMP3, MMP7, MMP9, PLOD2; Angiogenesis group: VEGFC, VEGFA, PDGFC, KDR, CDH5, VEGFB, PGF, TEK, ANGPT2, CXCR2, FLT1, CXCL8, VWF, ANGPT1, CXCL5; Endothelium group: KDR, CDH5, NOS3, VCAM1, VWF, FLT1, MMRN1, CLEC14A, ENG, MMRN2; Proliferation rate group: CCND1, CCNB1, CETN3, CDK2, E2F1, AURKA, BUB1, AURKB, PLK1, MCM6, ESCO2, MYBL2, MKI67, MCM2, CCNE1; and Epithelial to mesenchymal transition group: CDH2, ZEB1, ZEB2, TWIST1, SNAI1, SNAI2, TWIST2; Luminal differentiation group: PWRN1, PWRN3, GSTM5, GSTM4, GSTM2, ZNF321P, ZNF320, ZNF66, ZNF737, KRT20, UPK1B, FOXA1, ACER2, SEMA5A, PPARG, GATA3, SNX31, UPK2, UPK1A; Basal differentiation group: TM4SF19, SERPINB13, SERPINB3, SERPINB4, SPRR2F, SPRR2E, SPRR2A, SPRR2D, KRT17, KRT16, KRT14, DSG3, KRT5, KRT6C, KRT6A, KRT6B; Neuroendocrine differentiation group: PLEKHG4B, GNG4, PEG10, SOX2, TUBB2B, CHGB, SYP, ENO2, SV2A, MSI1, RND2, APLP1; and FGFR3 co-expressed group: FGFR3, TP63, IRS1, WNT7B, CAPNS2, ZNF385A, SMAD3, SLC2A9, DUOXA1, SYTL1, SEMA4B, CLCA4, PLCH2, SSH3, PTPN13, TMPRSS4.

In some embodiments, determining the UC TME signature comprises: determining gene group scores using the RNA expression levels for all genes in each of the following gene groups: MHC type I group: HLA-C, TAPBP, HLA-B, B2M, TAP2, HLA-A, TAP1, NLRC5; MHC type II group: HLA-DQB1, HLA-DMA, HLA-DMB, HLA-DRA, CIITA, HLA-DQA1, HLA-DPB1, HLA-DRB1, HLA-DPA1; Coactivation molecules group: TNFRSF4, CD27, CD80, CD40LG, TNFRSF9, CD40, CD28, ICOSLG, CD83, TNFSF9, CD70, TNFSF4, ICOS, CD86; Effector cells group: ZAP70, GZMB, GZMK, IFNG, FASLG, EOMES, TBX21, GZMA, CD8A, GNLY, PRF1, CD8B; Natural killer cells group: NKG7, FGFBP2, CD244, KLRK1, KIR2DL4, CD226, KLRF1, GNLY, GZMB, KLRC2, NCR1, GZMH, IFNG, SH2D1B, NCR3, EOMES, CD160; T cells group: TRBC2, CD3E, CD3G, ITK, CD28, TRBC1, TRAT1, TBX21, CD5, TRAC, CD3D; T-helper cells type 1 group: IL21, TBX21, IL12RB2, CD40LG, IFNG, IL2, STAT4; T-helper cells type 2 group: IL13, CCR4, IL10, IL4, IL5; B cells group: CD22, TNFRSF13C, STAP1, CD79B, PAX5, CR2, TNFRSF13B, CD79A, TNFRSF17, FCRL5, MS4A1, CD19, BLK; Macrophages group: MRC1, SIGLEC1, MSR1, CD163, CSF1R, CD68, IL4I1, IL10; Macrophages type 1 group: CMKLR1, SOCS3, IRF5, NOS2, IL1B, IL12B, IL23A, TNF, IL12A; Antitumor cytokines group: CCL3, IL21, IFNB1, IFNA2, TNF, TNFSF10; Checkpoint inhibition group: PDCD1, BTLA, HAVCR2, CD274, VSIR, LAG3, TIGIT, PDCD1LG2, CTLA4; T-regulatory cells group: IKZF2, TNFRSF18, IL10, FOXP3, CCR8, IKZF4, CTLA4; Neutrophils group: CD177, FFAR2, PGLYRP1, CXCR1, MPO, CXCR2, ELANE, CTSG, PRTN3, FCGR3B; MDSC group: ARG1, IL6, CYBB, IL10, PTGS2, IDO1, IL4I1; Protumor cytokines group: TGFB2, MIF, IL6, TGFB3, TGFB1, IL22, IL10; Cancer associated fibroblasts (CAF) group: COL6A3, PDGFRB, COL6A1, MFAP5, COL5A1, FAP, PDGFRA, FGF2, ACTA2, COL6A2, FBLN1, CD248, COL1A1, MMP2, COL1A2, MMP3, LUM, CXCL12, LRP1; Matrix group: LAMC2, TNC, COL11A1, VTN, LAMB3, COL1A1, FN1, LAMA3, LGALS9, COL1A2, COL4A1, COL5A1, ELN, LGALS7, COL3A1; Matrix remodeling group: ADAMTS4, ADAMTS5, CA9, LOX, MMP1, MMP11, MMP12, MMP2, MMP3, MMP7, MMP9, PLOD2; Angiogenesis group: VEGFC, VEGFA, PDGFC, KDR, CDH5, VEGFB, PGF, TEK, ANGPT2, CXCR2, FLT1, CXCL8, VWF, ANGPT1, CXCL5; Endothelium group: KDR, CDH5, NOS3, VCAM1, VWF, FLT1, MMRN1, CLEC14A, ENG, MMRN2; Proliferation rate group: CCND1, CCNB1, CETN3, CDK2, E2F1, AURKA, BUB1, AURKB, PLK1, MCM6, ESCO2, MYBL2, MKI67, MCM2, CCNE1; and Epithelial to mesenchymal transition group: CDH2, ZEB1, ZEB2, TWIST1, SNAI1, SNAI2, TWIST2; Luminal differentiation group: PWRN1, PWRN3, GSTM5, GSTM4, GSTM2, ZNF321P, ZNF320, ZNF66, ZNF737, KRT20, UPK1B, FOXA1, ACER2, SEMA5A, PPARG, GATA3, SNX31, UPK2, UPK1A; Basal differentiation group: TM4SF19, SERPINB13, SERPINB3, SERPINB4, SPRR2F, SPRR2E, SPRR2A, SPRR2D, KRT17, KRT16, KRT14, DSG3, KRT5, KRT6C, KRT6A, KRT6B; Neuroendocrine differentiation group: PLEKHG4B, GNG4, PEG10, SOX2, TUBB2B, CHGB, SYP, ENO2, SV2A, MSI1, RND2, APLP1; and FGFR3 co-expressed group: FGFR3, TP63, IRS1, WNT7B, CAPNS2, ZNF385A, SMAD3, SLC2A9, DUOXA1, SYTL1, SEMA4B, CLCA4, PLCH2, SSH3, PTPN13, TMPRSS4.

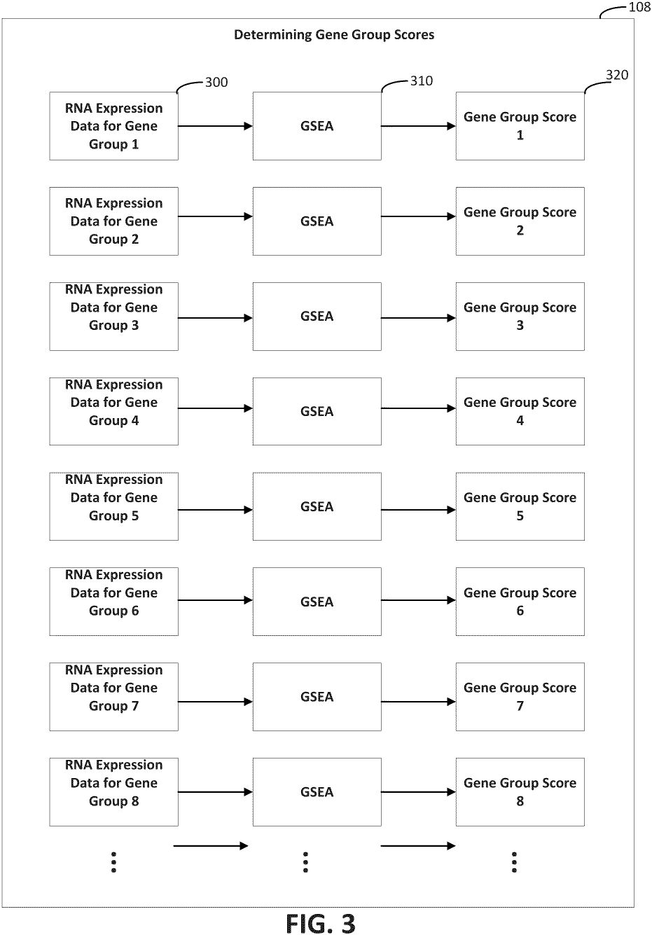

Aspects of determining the gene group scores are described herein, including with reference to FIG. 3 and in the Section titled “Gene Expression Signatures”.

As described above, at act 110, the UC TME signature is generated. In some embodiments, the UC TME signature consists of only gene group scores for one or more (e.g., all) of the gene groups listed in Table 1. In some embodiments, the UC TME signature comprises gene group scores for at least 2, 3, 4, 5, 6, 7, 8, 9, 10, 11, 12, 13, 14, 15, 16, 17, 18, 19, 20, 21, 22, 23, 24, 25, 26, 27, or 28 gene groups listed in Table 1. In some embodiments, each gene group score for a particular gene group is determined using RNA expression levels of some or all (e.g., at least 2, 3, 4, 5, 6, 7, 8, 9, 10, 11, 12, 13, 14, 15, 16, 17, 18, 19, 20, etc.) of the genes of each gene group listed in Table 1. In other embodiments, the UC TME signature includes one or more other gene group scores in addition to the gene group scores listed in Table 1.

Next, process 100 proceeds to act 112, where a UC TME type is identified for the subject using the UC TME signature generated at act 110. This may be done in any suitable way. For example, in some embodiments, the each of the possible UC TME types is associated with a respective plurality of UC TME signature clusters. In such embodiments, a UC TME type for the subject may be identified by associating the UC TME signature of the subject with a particular one of the plurality of UC TME signature clusters; and identifying the UC TME type for the subject as the UC TME type corresponding to the particular one of the plurality of UC TME signature clusters to which the UC TME signature of the subject is associated. Examples of UC TME types are described herein. Aspects of identifying a UC TME type for a subject are described herein including in the section below titled “Generating TME Signature and Identifying TME Type.”

As described above, a subject’s UC TME type is identified at act 112. In some embodiments, the UC TME type of a subject is identified to be one of the following UC TME types: Immune Desert (D) type, Immune Enriched (IE) type, Fibrotic (F) type, Immune Enriched -Fibrotic (IE/F) type, Immune Desert type, FGFR-altered (D/FGFR) type, Fibrotic - Basal (Bas) type, and Neuroendocrine-like (NE) type.

Optionally, process 100 proceeds to act 114, where the subject’s likelihood of responding to a therapy is identified using the UC TME type identified at act 112. In some embodiments, when a subject is identified as having a UC TME type IE, IE/F, or NE-like at act 112, the subject is identified as having an increased likelihood of responding to an immunotherapy (e.g., an anti-PD-L1 antibody, such as atezolizumab) relative to a subject having other UC TME types, at act 114. In some embodiments, when a subject is identified as having a UC TME type D at act 112, the subject is identified as having an increased likelihood of responding to an anti-FGRF3 therapy relative to a subject having other UC TME types, at act 114. In some embodiments, when a subject is identified as having a UC TME type D at act 112, the subject is identified as having an increased likelihood of responding to a PARP inhibitor or an ERBB2 inhibitor relative to a subject having other UC TME types, at act 114. In some embodiments, when a subject is identified as having a UC TME type F at act 112, the subject is identified as having an increased likelihood of responding to a PARP inhibitor or a TGF-beta inhibitor relative to a subject having other UC TME types, at act 114. In some embodiments, when a subject is identified as having a UC TME type Bas at act 112, the subject is identified as having an increased likelihood of responding to chemotherapy or radiotherapy relative to therapy with an ICI, at act 114. Aspects of identifying whether or not a subject is likely to respond to a therapy are described herein including in the section below titled “Therapeutic Indications.”

In some embodiments, process 100 completes after act 112 completes. In some such embodiments, the determined UC TME signature and/or identified UC TME type, and/or the identified likelihood the subject will respond to a therapy may be stored for subsequent use, provided to one or more recipients (e.g., a clinician, a researcher, etc.), and/or used to update the UC TME signature clusters (as described herein below).

However, in some embodiments, one or more other acts are performed after act 112. For example, in the illustrated embodiment of FIG. 1, process 100 may include one or more of optional acts 114, 116, and 118 shown using dashed lines in FIG. 1. For example, at act 116, a prognosis may be identified for the subject. In another example, when a subject is identified as having an increased likelihood of responding to immunotherapy at act 114, and/or having a particular prognosis at act 116, the subject is administered one or more immunotherapies at act 118. Examples of immunotherapies and other therapies are provided herein.

It should be appreciated that although acts 114, 116, and 118 are indicated as optional in the example of FIG. 1, in other embodiments, one or more other acts may be optional (in addition to or instead of acts 114, 116, and 118). For example, in some embodiments, acts 102 and 104 may be optional (e.g., when the sequencing data is obtained and processed to obtain RNA expression data previously, process 100 may begin at act 106 by accessing the previously obtained RNA expression data). In some embodiments, the process 100 may comprise acts 102, 104, 106, 114 and 118, without act 116. In some embodiments, the process 100 may comprise acts 102, 104, 106, 116, and 118, without act 114.

TABLE 1

Gene groups used to generate UC TME Signatures

| | |
|---|---|
| MHC type I | HLA-C, TAPBP, HLA-B, B2M, TAP2, HLA-A, TAP1, NLRC5 |
| MHC type II | HLA-DQB1, HLA-DMA, HLA-DMB, HLA-DRA, CIITA, HLA-DQA1, HLA-DPB1, HLA-DRB1, HLA-DPA1 |
| Coactivation molecules | TNFRSF4, CD27, CD80, CD40LG, TNFRSF9, CD40, CD28, ICOSLG, CD83, TNFSF9, CD70, TNFSF4, ICOS, CD86 |
| Effector cells | ZAP70, GZMB, GZMK, IFNG, FASLG, EOMES, TBX21, GZMA, CD8A, GNLY, |
| | PRF1, CD8B |
| Natural killer cells | NKG7, FGFBP2, CD244, KLRK1, KIR2DL4, CD226, KLRF1, GNLY, GZMB, KLRC2, NCR1, GZMH, IFNG, SH2D1B, NCR3, EOMES, CD160 |
| T cells | TRBC2, CD3E, CD3G, ITK, CD28, TRBC1, TRAT1, TBX21, CD5, TRAC, CD3D |
| T-helper cells type 1 | IL21, TBX21, IL12RB2, CD40LG, IFNG, IL2, STAT4 |
| T-helper cells type 2 | IL13, CCR4, IL10, IL4, IL5 |
| B cells | CD22, TNFRSF13C, STAP1, CD79B, PAX5, CR2, TNFRSF13B, CD79A, TNFRSF17, FCRL5, MS4A1, CD19, BLK |
| Macrophages | MRC1, SIGLEC1, MSR1, CD163, CSF1R, CD68, IL411, IL10 |
| Macrophages type 1 | CMKLR1, SOCS3, IRF5, NOS2, IL1B, IL12B, IL23A, TNF, IL12A |
| Antitumor cytokines | CCL3, IL21, IFNB1, IFNA2, TNF, TNFSF10 |
| Checkpoint inhibition | PDCD1, BTLA, HAVCR2, CD274, VSIR, LAG3, TIGIT, PDCD1LG2, CTLA4 |
| T-regulatory cells | IKZF2, TNFRSF18, IL10, FOXP3, CCR8, IKZF4, CTLA4 |
| Neutrophils | CD177, FFAR2, PGLYRP1, CXCR1, MPO, CXCR2, ELANE, CTSG, PRTN3, FCGR3B |
| MDSC | ARG1, IL6, CYBB, IL10, PTGS2, IDO1, IL411 |
| Protumor cytokines | TGFB2, MIF, IL6, TGFB3, TGFB1, IL22, IL10 |
| CAF | COL6A3, PDGFRB, COL6A1, MFAP5, COL5A1, FAP, PDGFRA, FGF2, ACTA2, COL6A2, FBLN1, CD248, COL1A1, MMP2, COL1A2, MMP3, LUM, CXCL12, LRP1 |
| Matrix | LAMC2, TNC, COL11A1, VTN, LAMB3, COL1A1, FN1, LAMA3, LGALS9, COL1A2, COL4A 1, COL5A1, ELN, LGALS7, COL3A 1 |
| Matrix remodeling | ADAMTS4, ADAMTS5, CA9, LOX, MMP1, MMP11, MMP12, MMP2, MMP3, MMP7, MMP9, PLOD2 |
| Angiogenesis | VEGFC, VEGFA, PDGFC, KDR, CDH5, VEGFB, PGF, TEK, ANGPT2, CXCR2, FLT1, CXCL8, VWF, ANGPT1, CXCL5 |
| Endothelium | KDR, CDH5, NOS3, VCAM1, VWF, FLT1, MMRN1, CLEC14A, ENG, MMRN2 |
| Proliferation_rate | CCND1, CCNB1, CETN3, CDK2, E2F1, AURKA, BUB1, AURKB, PLK1, MCM6, ESCO2, MYBL2, MKI67, MCM2, CCNE1 |
| Epithelial to mesenchymal transition | CDH2, ZEB1, ZEB2, TWIST1, SNAI1, SNAI2, TWIST2 |
| Luminal differentiation | PWRN1, PWRN3, GSTM5, GSTM4, GSTM2, ZNF321P, ZNF320, ZNF66, ZNF737, KRT20, UPK1B, FOXA1, ACER2, SEMA5A, PPARG, GATA3, SNX31, UPK2, UPK1A |
| Basal differentiation | TM4SF19, SERPINB13, SERPINB3, SERPINB4, SPRR2F, SPRR2E, SPRR2A, SPRR2D, KRT17, KRT16, KRT14, DSG3, KRT5, KRT6C, KRT6A, KRT6B |
| Neuroendocrine differentiation | PLEKHG4B, GNG4, PEG10, SOX2, TUBB2B, CHGB, SYP, ENO2, SV2A, MSI1, RND2, APLP1 |
| FGFR3 co-expressed | FGFR3, TP63, IRS1, WNT7B, CAPNS2, ZNF385A, SMAD3, SLC2A9, DUOXA1, SYTL1, SEMA4B, CLCA4, PLCH2, SSH3, PTPN13, TMPRSS4 |

### Biological Samples

Aspects of the disclosure relate to methods for identifying the urothelial cancer type (UCT) of a subject by analyzing gene expression data obtained from a biological sample that has been obtained from the subject.

The biological sample may be from any source in the subject’s body including, but not limited to, any fluid [such as blood (e.g., whole blood, blood serum, or blood plasma), saliva, tears, synovial fluid, cerebrospinal fluid, pleural fluid, pericardial fluid, ascitic fluid, and/or urine], hair, skin (including portions of the epidermis, dermis, and/or hypodermis), oropharynx, laryngopharynx, esophagus, stomach, bronchus, salivary gland, tongue, oral cavity, nasal cavity, vaginal cavity, anal cavity, bone, bone marrow, brain, thymus, spleen, small intestine, appendix, colon, rectum, anus, liver, biliary tract, pancreas, kidney, ureter, bladder, urethra, uterus, vagina, vulva, ovary, cervix, scrotum, penis, prostate, testicle, seminal vesicles, and/or any type of tissue (e.g., muscle tissue, epithelial tissue, connective tissue, or nervous tissue). In some embodiments, the tissue sample comprises a bladder or urothelial tissue sample.

The biological sample may be any type of sample including, for example, a sample of a bodily fluid, one or more cells, a piece of tissue, or some or all of an organ. In some embodiments, a tissue sample may be obtained from a subject using a surgical procedure (e.g., laparoscopic surgery, microscopically controlled surgery, or endoscopy), bone marrow biopsy, punch biopsy, endoscopic biopsy, or needle biopsy (e.g., a fine-needle aspiration, core needle biopsy, vacuum-assisted biopsy, or image-guided biopsy).

A sample of lymph node or blood, in some embodiments, refers to a sample comprising cells, e.g., cells from a blood sample or lymph node sample. In some embodiments, the sample comprises non-cancerous cells. In some embodiments, the sample comprises pre-cancerous cells. In some embodiments, the sample comprises cancerous cells. In some embodiments, the sample comprises blood cells. In some embodiments, the sample comprises lymph node cells. In some embodiments, the sample comprises lymph node cells and blood cells.

A sample of blood may be a sample of whole blood or a sample of fractionated blood. In some embodiments, the sample of blood comprises whole blood. In some embodiments, the sample of blood comprises fractionated blood. In some embodiments, the sample of blood comprises buffy coat. In some embodiments, the sample of blood comprises serum. In some embodiments, the sample of blood comprises plasma. In some embodiments, the sample of blood comprises a blood clot.

In some embodiments, a sample of blood is collected to obtain the cell-free nucleic acid (e.g., cell-free DNA) in the blood.

In some embodiments, the sample may be from a cancerous tissue or organ or a tissue or organ suspected of having one or more cancerous cells. In some embodiments, the sample may be from a healthy (e.g., non-cancerous) tissue or organ. In some embodiments, a sample from a subject (e.g., a biopsy from a subject) may include both healthy and cancerous cells and/or tissue. In certain embodiments, one sample will be taken from a subject for analysis. In some embodiments, more than one (e.g., 2, 3, 4, 5, 6, 7, 8, 9, 10, 11, 12, 13, 14, 15, 16, 17, 18, 19, 20, or more) samples may be taken from a subject for analysis. In some embodiments, one sample from a subject will be analyzed. In certain embodiments, more than one (e.g., 2, 3, 4, 5, 6, 7, 8, 9, 10, 11, 12, 13, 14, 15, 16, 17, 18, 19, 20, or more) samples may be analyzed. If more than one sample from a subject is analyzed, the samples may be procured at the same time (e.g., more than one sample may be taken in the same procedure), or the samples may be taken at different times (e.g., during a different procedure including a procedure 1, 2, 3, 4, 5, 6, 7, 8, 9, 10 days; 1, 2, 3, 4, 5, 6, 7, 8, 9, 10 weeks; 1, 2, 3, 4, 5, 6, 7, 8, 9, 10 months, 1, 2, 3, 4, 5, 6, 7, 8, 9, 10 years, or 1, 2, 3, 4, 5, 6, 7, 8, 9, 10 decades after a first procedure). A second or subsequent sample may be taken or obtained from the same region (e.g., from the same tumor or area of tissue) or a different region (including, e.g., a different tumor). A second or subsequent sample may be taken or obtained from the subject after one or more treatments, and may be taken from the same region or a different region. As a non-limiting example, the second or subsequent sample may be useful in determining whether the cancer in each sample has different characteristics (e.g., in the case of samples taken from two physically separate tumors in a patient) or whether the cancer has responded to one or more treatments (e.g., in the case of two or more samples from the same tumor prior to and subsequent to a treatment).

Any of the biological samples described herein may be obtained from the subject using any known technique. In some embodiments, the biological sample may be obtained from a surgical procedure (e.g., laparoscopic surgery, microscopically controlled surgery, or endoscopy), bone marrow biopsy, punch biopsy, endoscopic biopsy, or needle biopsy (e.g., a fine-needle aspiration, core needle biopsy, vacuum-assisted biopsy, or image-guided biopsy). In some embodiments, each of the at least one biological sample is a bodily fluid sample, a cell sample, or a tissue biopsy.

Any of the biological samples from a subject described herein may be stored using any method that preserves stability of the biological sample. In some embodiments, preserving the stability of the biological sample means inhibiting components (e.g., DNA, RNA, protein, or tissue structure or morphology) of the biological sample from degrading until they are measured so that when measured, the measurements represent the state of the sample at the time of obtaining it from the subject. In some embodiments, a biological sample is stored in a composition that is able to penetrate the same and protect components (e.g., DNA, RNA, protein, or tissue structure or morphology) of the biological sample from degrading. As used herein, degradation is the transformation of a component from one form to another form such that the first form is no longer detected at the same level as before degradation.

In some embodiments, the biological sample is stored using cryopreservation. Non-limiting examples of cryopreservation include, but are not limited to, step-down freezing, blast freezing, direct plunge freezing, snap freezing, slow freezing using a programmable freezer, and vitrification. In some embodiments, the biological sample is stored using lyophilization. In some embodiments, a biological sample is placed into a container that already contains a preservant (e.g., RNALater to preserve RNA) and then frozen (e.g., by snap-freezing), after the collection of the biological sample from the subject. In some embodiments, such storage in frozen state is done immediately after collection of the biological sample. In some embodiments, a biological sample may be kept at either room temperature or 4° C. for some time (e.g., up to an hour, up to 8 h, or up to 1 day, or a few days) in a preservant or in a buffer without a preservant, before being frozen.

Non-limiting examples of preservants include formalin solutions, formaldehyde solutions, RNALater or other equivalent solutions, TriZol or other equivalent solutions, DNA/RNA Shield or equivalent solutions, EDTA (e.g., Buffer AE (10 mM Tris·Cl; 0.5 mM EDTA, pH 9.0)) and other coagulants, and Acids Citrate Dextrose (e.g., for blood specimens).

In some embodiments, special containers may be used for collecting and/or storing a biological sample. For example, a vacutainer may be used to store blood. In some embodiments, a vacutainer may comprise a preservant (e.g., a coagulant, or an anticoagulant). In some embodiments, a container in which a biological sample is preserved may be contained in a secondary container, for the purpose of better preservation, or for the purpose of avoid contamination.

Any of the biological samples from a subject described herein may be stored under any condition that preserves stability of the biological sample. In some embodiments, the biological sample is stored at a temperature that preserves stability of the biological sample. In some embodiments, the sample is stored at room temperature (e.g., 25° C.). In some embodiments, the sample is stored under refrigeration (e.g., 4° C.). In some embodiments, the sample is stored under freezing conditions (e.g., -20° C.). In some embodiments, the sample is stored under ultralow temperature conditions (e.g., -50° C. to -800° C.). In some embodiments, the sample is stored under liquid nitrogen (e.g., -1700° C.). In some embodiments, a biological sample is stored at -60° C. to -8° C.(e.g., -70° C.) for up to 5 years (e.g., up to 1 month, up to 2 months, up to 3 months, up to 4 months, up to 5 months, up to 6 months, up to 7 months, up to 8 months, up to 9 months, up to 10 months, up to 11 months, up to 1 year, up to 2 years, up to 3 years, up to 4 years, or up to 5 years). In some embodiments, a biological sample is stored as described by any of the methods described herein for up to 20 years (e.g., up to 5 years, up to 10 years, up to 15 years, or up to 20 years).

### Obtaining RNA Expression Data

Aspects of the disclosure relate to methods of determining a urothelial cancer TME type of a subject using sequencing data or RNA expression data obtained from a biological sample from the subject.

The RNA expression data used in methods described herein typically is derived from sequencing data obtained from the biological sample.

The sequencing data may be obtained from the biological sample using any suitable sequencing technique and/or apparatus. In some embodiments, the sequencing apparatus used to sequence the biological sample may be selected from any suitable sequencing apparatus known in the art including, but not limited to, Illumina™, SOLid™, Ion Torrent™, PacBio™, a nanopore-based sequencing apparatus, a Sanger sequencing apparatus, or a 454™ sequencing apparatus. In some embodiments, sequencing apparatus used to sequence the biological sample is an Illumina sequencing (e.g., NovaSeq™, NextSeq™, HiSeq™, MiSeq™, or MiniSeq™) apparatus.

After the sequencing data is obtained, it is processed in order to obtain the RNA expression data. RNA expression data may be acquired using any method known in the art including, but not limited to whole transcriptome sequencing, whole exome sequencing, total RNA sequencing, mRNA sequencing, targeted RNA sequencing, RNA exome capture sequencing, next generation sequencing, and/or deep RNA sequencing. In some embodiments, RNA expression data may be obtained using a microarray assay.

In some embodiments, the sequencing data is processed to produce RNA expression data. In some embodiments, RNA sequence data is processed by one or more bioinformatics methods or software tools, for example RNA sequence quantification tools (e.g., Kallisto) and genome annotation tools (e.g., Gencode v23), in order to produce expression data. The Kallisto software is described in Nicolas L Bray, Harold Pimentel, Páll Melsted and Lior Pachter, Near-optimal probabilistic RNA-seq quantification, Nature Biotechnology 34, 525-527 (2016), doi:10.1038/nbt.3519, which is incorporated by reference in its entirety herein.

In some embodiments, microarray expression data is processed using a bioinformatics R package, such as “affy” or “limma,” in order to produce expression data. The “affy” software is described in Bioinformatics. 2004 Feb 12;20(3):307-15. doi: 10.1093/bioinformatics/btg405. “affy--analysis of Affymetrix GeneChip data at the probe level” by Laurent Gautier 1, Leslie Cope, Benjamin M Bolstad, Rafael A Irizarry PMID: 14960456 DOI: 10.1093/bioinformatics/btg405, which is incorporated by reference herein in its entirety. The “limma” software is described in Ritchie ME, Phipson B, Wu D, Hu Y, Law CW, Shi W, Smyth GK “limma powers differential expression analyses for RNA-sequencing and microarray studies.” Nucleic Acids Res. 2015 Apr 20;43(7):e47. 20. doi.org/10.1093/nar/gkv007PMID: 25605792, PMCID: PMC4402510, which is incorporated by reference herein its entirety.

In some embodiments, sequencing data and/or expression data comprises more than 5 kilobases (kb). In some embodiments, the size of the obtained RNA data is at least 10 kb. In some embodiments, the size of the obtained RNA sequencing data is at least 100 kb. In some embodiments, the size of the obtained RNA sequencing data is at least 500 kb. In some embodiments, the size of the obtained RNA sequencing data is at least 1 megabase (Mb). In some embodiments, the size of the obtained RNA sequencing data is at least 10 Mb. In some embodiments, the size of the obtained RNA sequencing data is at least 100 Mb. In some embodiments, the size of the obtained RNA sequencing data is at least 500 Mb. In some embodiments, the size of the obtained RNA sequencing data is at least 1 gigabase (Gb). In some embodiments, the size of the obtained RNA sequencing data is at least 10 Gb. In some embodiments, the size of the obtained RNA sequencing data is at least 100 Gb. In some embodiments, the size of the obtained RNA sequencing data is at least 500 Gb.

In some embodiments, the expression data is acquired through bulk RNA sequencing. Bulk RNA sequencing may include obtaining expression levels for each gene across RNA extracted from a large population of input cells (e.g., a mixture of different cell types.) In some embodiments, the expression data is acquired through single cell sequencing (e.g., scRNA-seq). Single cell sequencing may include sequencing individual cells.

In some embodiments, bulk sequencing data comprises at least 1 million reads, at least 5 million reads, at least 10 million reads, at least 20 million reads, at least 50 million reads, or at least 100 million reads. In some embodiments, bulk sequencing data comprises between 1 million reads and 5 million reads, 3 million reads and 10 million reads, 5 million reads and 20 million reads, 10 million reads and 50 million reads, 30 million reads and 100 million reads, or 1 million reads and 100 million reads (or any number of reads including, and between).

In some embodiments, the expression data comprises next-generation sequencing (NGS) data. In some embodiments, the expression data comprises microarray data.

Expression data (e.g., indicating expression levels) for a plurality of genes may be used for any of the methods or compositions described herein. The number of genes which may be examined may be up to and inclusive of all the genes of the subject. In some embodiments, expression levels may be determined for all of the genes of a subject. As a non-limiting example, four or more, five or more, six or more, seven or more, eight or more, nine or more, ten or more, eleven or more, twelve or more, 13 or more, 14 or more, 15 or more, 16 or more, 17 or more, 18 or more, 19 or more, 20 or more, 21 or more, 22 or more, 23 or more, 24 or more, 25 or more, 26 or more, 27 or more, 28 or more, 29 or more, 30 or more, 35 or more, 40 or more, 50 or more, 60 or more, 70 or more, 80 or more, 90 or more, 100 or more, 125 or more, 150 or more, 175 or more, 200 or more, 225 or more, 250 or more, 275 or more, or 300 or more genes may be used for any evaluation described herein. As another set of non-limiting examples, the expression data may include, for each gene group listed in Table 1, expression data for at least 5, at least 10, at least 15, at least 20, or at least 25 genes selected from each gene group.

In some embodiments, RNA expression data is obtained by accessing the RNA expression data from at least one computer storage medium on which the RNA expression data is stored. Additionally or alternatively, in some embodiments, RNA expression data may be received from one or more sources via a communication network of any suitable type. For example, in some embodiment, the RNA expression data may be received from a server (e.g., a SFTP server, or Illumina BaseSpace).

The RNA expression data obtained may be in any suitable format, as aspects of the technology described herein are not limited in this respect. For example, in some embodiments, the RNA expression data may be obtained in a text-based file (e.g., in a FASTQ, FASTA, BAM, or SAM format). In some embodiments, a file in which sequencing data is stored may contains quality scores of the sequencing data. In some embodiments, a file in which sequencing data is stored may contain sequence identifier information.

Expression data, in some embodiments, includes gene expression levels. Gene expression levels may be detected by detecting a product of gene expression such as mRNA and/or protein. In some embodiments, gene expression levels are determined by detecting a level of a mRNA in a sample. As used herein, the terms “determining” or “detecting” may include assessing the presence, absence, quantity and/or amount (which can be an effective amount) of a substance within a sample, including the derivation of qualitative or quantitative concentration levels of such substances, or otherwise evaluating the values and/or categorization of such substances in a sample from a subject.

FIG. 2 shows an exemplary process 104 for processing sequencing data to obtain RNA expression data from sequencing data. Process 104 may be performed by any suitable computing device or devices, as aspects of the technology described herein are not limited in this respect. For example, process 104 may be performed by a computing device part of a sequencing apparatus. In other embodiments, process 104 may be performed by one or more computing devices external to the sequencing apparatus.

Process 104 begins at act 200, where sequencing data is obtained from a biological sample obtained from a subject. The sequencing data is obtained by any suitable method, for example, using any of the methods described herein including in the Section titled “Biological Samples.”

In some embodiments, the sequencing data obtained at act 200 comprises RNA-seq data. In some embodiments, the biological sample comprises blood or tissue. In some embodiments, the biological sample comprises one or more tumor cells, for example, one or more bladder tumor cells.

Next, process 104 proceeds to act 202 where the sequencing data obtained at act 200 is normalized to transcripts per kilobase million (TPM) units. The normalization may be performed using any suitable software and in any suitable way. For example, in some embodiments, TPM normalization may be performed according to the techniques described in Wagner et al. (Theory Biosci. (2012) 131:281-285), which is incorporated by reference herein in its entirety. In some embodiments, the TPM normalization may be performed using a software package, such as, for example, the gcrma package. Aspects of the gcrma package are described in Wu J, Gentry RIwcfJMJ (2021). “gcrma: Background Adjustment Using Sequence Information. R package version 2.66.0.,” which is incorporated by reference in its entirety herein. In some embodiments, RNA expression level in TPM units for a particular gene may be calculated according to the following formula:

$$\begin{matrix}A ⋅ \frac{1}{\sum \left(A\right)} ⋅ {10}^{6} \\ W h e r e A = \frac{t o t a l r e a d s m a p p e d t o g e n e ⋅ {10}^{3}}{g e n e l e n g t h i n b p}\end{matrix}$$

Next, process 104 proceeds to act 204, where the RNA expression levels in TPM units (as determined at act 202) may be log transformed. Process 104 is illustrative and there are variations. For example, in some embodiments, one or both of acts 202 and 204 may be omitted. Thus, in some embodiments, the RNA expression levels may not be normalized to transcripts per million units and may, instead, be converted to another type of unit (e.g., reads per kilobase million (RPKM) or fragments per kilobase million (FPKM) or any other suitable unit). Additionally or alternatively, in some embodiments, the log transformation may be omitted. Instead, no transformation may be applied in some embodiments, or one or more other transformations may be applied in lieu of the log transformation.

RNA expression data obtained by process 104 can include the sequence data generated by a sequencing protocol (e.g., the series of nucleotides in a nucleic acid molecule identified by next-generation sequencing, sanger sequencing, etc.) as well as information contained therein (e.g., information indicative of source, tissue type, etc.) which may also be considered information that can be inferred or determined from the sequence data. In some embodiments, expression data obtained by process 104 can include information included in a FASTA file, a description and/or quality scores included in a FASTQ file, an aligned position included in a BAM file, and/or any other suitable information obtained from any suitable file.

### Urothelial Cancer Signatures

Aspects of the disclosure relate to processing of expression data to determine one or more gene expression signatures (e.g., a urothelial cancer TME signature). In some embodiments, expression data (e.g., RNA expression data) is processed using a computing device to determine the one or more gene expression signatures. In some embodiments, the computing device may be operated by a user such as a doctor, clinician, researcher, patient, or other individual. For example, the user may provide the expression data as input to the computing device (e.g., by uploading a file), and/or may provide user input specifying processing or other methods to be performed using the expression data.

In some embodiments, expression data may be processed by one or more software programs running on computing device.

In some embodiments, methods described herein comprise an act of determining a UC TME signature comprising gene group scores for respective gene groups in a plurality of gene groups. In some embodiments, a UC TME signature comprises gene group scores for at least one (e.g., 1, 2, 3, 4, 5, 6, 7, 8, 9, 10, 11, 12, 13, 14, 15, 16, 17, 18, 19, 20, 21, 22, 23, 24, 25, 26, 27, or 28) of the gene groups listed in Table 1.

The number of genes in a gene group used to determine a gene group score may vary. In some embodiments, all RNA expression levels for all genes in a particular gene group may be used to determine a gene group score for the particular gene group. In other embodiments, RNA expression data for fewer than all genes may be used (e.g., RNA expression levels for at least two genes, at least three genes, at least five genes, between 2 and 10 genes, between 5 and 15 genes, between 3 and 20 genes, or any other suitable range within these ranges).

In some embodiments, a TME signature comprises a gene group score for the MHC I group. In some embodiments, this gene group score may be calculated using RNA expression levels of at least three genes (e.g., at least 3, at least 4, at least 5, at least 6, or at least 7) in the MCH I group, which is defined by its constituent genes: HLA-C, TAPBP, HLA-B, B2M, TAP2, HLA-A, TAP1, and NLRC5.

In some embodiments, a TME signature comprises a gene group score for the MHC II group. In some embodiments, this gene group score may be calculated using RNA expression levels of at least three genes (e.g., at least 3, at least 4, at least 5, at least 6, at least 7, at least 8, or at least 9) in the MCH II group, which is defined by its constituent genes: HLA-DQB1, HLA-DMA, HLA-DMB, HLA-DRA, CIITA, HLA-DQA1, HLA-DPB1, HLA-DRB1, and HLA-DPA1.

In some embodiments, a TME signature comprises a gene group score for the Coactivation molecules group. In some embodiments, this gene group score may be calculated using RNA expression levels of at least three genes (e.g., at least 3, at least 4, at least 5, at least 6, at least 7, at least 8, at least 9, at least 10, at least 11, at least 12, at least 13, or at least 14) in the Coactivation molecules group, which is defined by its constituent genes: TNFRSF4, CD27, CD80, CD40LG, TNFRSF9, CD40, CD28, ICOSLG, CD83, TNFSF9, CD70, TNFSF4, ICOS, and CD86.

In some embodiments, a TME signature comprises a gene group score for the Effector cells group. In some embodiments, this gene group score may be calculated using RNA expression levels of at least three genes (e.g., at least 3, at least 4, at least 5, at least 6, at least 7, at least 8, at least 9, at least 10, at least 11, or at least 12) in the Effector cells group, which is defined by its constituent genes: ZAP70, GZMB, GZMK, IFNG, FASLG, EOMES, TBX21, GZMA, CD8A, GNLY, PRF1, and CD8B.

In some embodiments, a TME signature comprises a gene group score for the Natural killer cells group. In some embodiments, this gene group score may be calculated using RNA expression levels of at least three genes (e.g., at least 3, at least 4, at least 5, at least 6, at least 7, at least 8, at least 9, at least 10, at least 11, at least 12, at least 13, at least 14, at least 15, at least 16, or at least 17) in the Natural killer cells group, which is defined by its constituent genes: NKG7, FGFBP2, CD244, KLRK1, KIR2DL4, CD226, KLRF1, GNLY, GZMB, KLRC2, NCR1, GZMH, IFNG, SH2D1B, NCR3, EOMES, and CD160.

In some embodiments, a TME signature comprises a gene group score for the T cells group. In some embodiments, this gene group score may be calculated using RNA expression levels of at least three genes (e.g., at least 3, at least 4, at least 5, at least 6, at least 7, at least 8, at least 9, at least 10, or at least 11) in the T cells group, which is defined by its constituent genes: TRBC2, CD3E, CD3G, ITK, CD28, TRBC1, TRAT1, TBX21, CD5, TRAC, and CD3D.

In some embodiments, a TME signature comprises a gene group score for the T-helper cells type 1 group. In some embodiments, this gene group score may be calculated using RNA expression levels of at least three genes (e.g., at least 3, at least 4, at least 5, at least 6, or at least 7) in the T-helper cells type 1 group, which is defined by its constituent genes: IL21, TBX21, IL12RB2, CD40LG, IFNG, IL2, and STAT4.

In some embodiments, a TME signature comprises a gene group score for the T-helper cells type 2 group. In some embodiments, this gene group score may be calculated using RNA expression levels of at least three genes (e.g., at least 3, at least 4, or at least 5) in the T-helper cells type 2 group, which is defined by its constituent genes: IL13, CCR4, IL10, IL4, and IL5.

In some embodiments, a TME signature comprises a gene group score for the B cells group. In some embodiments, this gene group score may be calculated using RNA expression levels of at least three genes (e.g., at least 3, at least 4, at least 5, at least 6, at least 7, at least 8, at least 9, at least 10, at least 11, at least 12, or at least 13) in the B cells group, which is defined by its constituent genes: CD22, TNFRSF13C, STAP1, CD79B, PAX5, CR2, TNFRSF13B, CD79A, TNFRSF17, FCRL5, MS4A1, CD19, and BLK.

In some embodiments, a TME signature comprises a gene group score for the Macrophages group. In some embodiments, this gene group score may be calculated using RNA expression levels of at least three genes (e.g., at least 3, at least 4, at least 5, at least 6, at least 7, or at least 8) in the Macrophages group, which is defined by its constituent genes: MRC1, SIGLEC1, MSR1, CD163, CSF1R, CD68, IL4I1, and IL10.

In some embodiments, a TME signature comprises a gene group score for the Macrophages type 1 (M1) group. In some embodiments, this gene group score may be calculated using RNA expression levels of at least three genes (e.g., at least 3, at least 4, at least 5, at least 6, at least 7, at least 8, or at least 9) in the Macrophages type 1 (M1) group, which is defined by its constituent genes: CMKLR1, SOCS3, IRF5, NOS2, IL1B, IL12B, IL23A, TNF, and IL12A.

In some embodiments, a TME signature comprises a gene group score for the Antitumor cytokines group. In some embodiments, this gene group score may be calculated using RNA expression levels of at least three genes (e.g., at least 3, at least 4, at least 5, or at least 6) in the Antitumor cytokines group, which is defined by its constituent genes: CCL3, IL21, IFNB1, IFNA2, TNF, and TNFSF10.

In some embodiments, a TME signature comprises a gene group score for the Checkpoint inhibition group. In some embodiments, this gene group score may be calculated using RNA expression levels of at least three genes (e.g., at least 3, at least 4, at least 5, at least 6, at least 7, at least 8, or at least 9) in the Checkpoint inhibition group, which is defined by its constituent genes: PDCD1, BTLA, HAVCR2, CD274, VSIR, LAG3, TIGIT, PDCD1LG2, and CTLA4.

In some embodiments, a TME signature comprises a gene group score for the T-regulatory cells group. In some embodiments, this gene group score may be calculated using RNA expression levels of at least three genes (e.g., at least 3, at least 4, at least 5, at least 6, or at least 7) in the T-regulatory cells group, which is defined by its constituent genes: IKZF2, TNFRSF18, IL10, FOXP3, CCR8, IKZF4, and CTLA4.

In some embodiments, a TME signature comprises a gene group score for the Neutrophils group. In some embodiments, this gene group score may be calculated using RNA expression levels of at least three genes (e.g., at least 3, at least 4, at least 5, at least 6, at least 7, at least 8, at least 9, or at least 10) in the Neutrophils group, which is defined by its constituent genes: CD177, FFAR2, PGLYRP1, CXCR1, MPO, CXCR2, ELANE, CTSG, PRTN3, and FCGR3B.

In some embodiments, a TME signature comprises a gene group score for the MDSC group. In some embodiments, this gene group score may be calculated using RNA expression levels of at least three genes (e.g., at least 3, at least 4, at least 5, at least 6, or at least 7) in the MDSC group, which is defined by its constituent genes: ARG1, IL6, CYBB, IL10, PTGS2, IDO1, and IL4I1.

In some embodiments, a TME signature comprises a gene group score for the Protumor cytokines group. In some embodiments, this gene group score may be calculated using RNA expression levels of at least three genes (e.g., at least 3, at least 4, at least 5, at least 6, or at least 7) in the Protumor cytokines group, which is defined by its constituent genes: TGFB2, MIF, IL6, TGFB3, TGFB1, IL22, and IL10.

In some embodiments, a TME signature comprises a gene group score for the Cancer associated fibroblasts (CAF) group. In some embodiments, this gene group score may be calculated using RNA expression levels of at least three genes (e.g., at least 3, at least 4, at least 5, at least 6, at least 7, at least 8, at least 9, at least 10, at least 11, at least 12, at least 13, at least 14, at least 15, at least 16, at least 17, at least 18, or at least 19) in the Cancer associated fibroblasts (CAF) group, which is defined by its constituent genes: COL6A3, PDGFRB, COL6A1, MFAP5, COL5A1, FAP, PDGFRA, FGF2, ACTA2, COL6A2, FBLN1, CD248, COL1A1, MMP2, COL1A2, MMP3, LUM, CXCL12, and LRP1.

In some embodiments, a TME signature comprises a gene group score for the Matrix group. In some embodiments, this gene group score may be calculated using RNA expression levels of at least three genes (e.g., at least 3, at least 4, at least 5, at least 6, at least 7, at least 8, at least 9, at least 10, at least 11, at least 12, at least 13, at least 14, or at least 15) in the Matrix group, which is defined by its constituent genes: LAMC2, TNC, COL11A1, VTN, LAMB3, COL1A1, FN1, LAMA3, LGALS9, COL1A2, COL4A1, COL5A1, ELN, LGALS7, and COL3A1.

In some embodiments, a TME signature comprises a gene group score for the Matrix remodeling group. In some embodiments, this gene group score may be calculated using RNA expression levels of at least three genes (e.g., at least 3, at least 4, at least 5, at least 6, at least 7, at least 8, at least 9, at least 10, at least 11, or at least 12) in the Matrix remodeling group, which is defined by its constituent genes: ADAMTS4, ADAMTS5, CA9, LOX, MMP1, MMP11, MMP12, MMP2, MMP3, MMP7, MMP9, and PLOD2.

In some embodiments, a TME signature comprises a gene group score for the Angiogenesis group. In some embodiments, this gene group score may be calculated using RNA expression levels of at least three genes (e.g., at least 3, at least 4, at least 5, at least 6, at least 7, at least 8, at least 9, at least 10, at least 11, at least 12, at least 13, at least 14, or at least 15) in the Angiogenesis group, which is defined by its constituent genes: VEGFC, VEGFA, PDGFC, KDR, CDH5, VEGFB, PGF, TEK, ANGPT2, CXCR2, FLT1, CXCL8, VWF, ANGPT1, and CXCL5.

In some embodiments, a TME signature comprises a gene group score for the Endothelium group. In some embodiments, this gene group score may be calculated using RNA expression levels of at least three genes (e.g., at least 3, at least 4, at least 5, at least 6, at least 7, at least 8, at least 9, or at least 10) in the Endothelium group, which is defined by its constituent genes: KDR, CDH5, NOS3, VCAM1, VWF, FLT1, MMRN1, CLEC14A, ENG, and MMRN2.

In some embodiments, a TME signature comprises a gene group score for the Proliferation rate group. In some embodiments, this gene group score may be calculated using RNA expression levels of at least three genes (e.g., at least 3, at least 4, at least 5, at least 6, at least 7, at least 8, at least 9, at least 10, at least 11, at least 12, at least 13, at least 14, or at least 15) in the Proliferation rate group, which is defined by its constituent genes: CCND1, CCNB1, CETN3, CDK2, E2F1, AURKA, BUB1, AURKB, PLK1, MCM6, ESCO2, MYBL2, MKI67, MCM2, and CCNE1.

In some embodiments, a TME signature comprises a gene group score for the Epithelial to mesenchymal transition (EMT) group. In some embodiments, this gene group score may be calculated using RNA expression levels of at least three genes (e.g., at least 3, at least 4, at least 5, at least 6, or at least 7) in the Epithelial to mesenchymal transition (EMT) group, which is defined by its constituent genes: CDH2, ZEB1, ZEB2, TWIST1, SNAI1, SNAI2, and TWIST2.

In some embodiments, a TME signature comprises a gene group score for the Luminal differentiation group. In some embodiments, this gene group score may be calculated using RNA expression levels of at least three genes (e.g., at least 3, at least 4, at least 5, at least 6, at least 7, at least 8, at least 9, at least 10, at least 11, at least 12, at least 13, at least 14, at least 15, at least 16, at least 17, at least 18, or at least 19) in the Luminal differentiation group, which is defined by its constituent genes: PWRN1, PWRN3, GSTM5, GSTM4, GSTM2, ZNF321P, ZNF320, ZNF66, ZNF737, KRT20, UPK1B, FOXA1, ACER2, SEMA5A, PPARG, GATA3, SNX31, UPK2, and UPK1A.

In some embodiments, a TME signature comprises a gene group score for the Basal differentiation group. In some embodiments, this gene group score may be calculated using RNA expression levels of at least three genes (e.g., at least 3, at least 4, at least 5, at least 6, at least 7, at least 8, at least 9, at least 10, at least 11, at least 12, at least 13, at least 14, at least 15, or at least 16) in the Basal differentiation group, which is defined by its constituent genes: TM4SF19, SERPINB13, SERPINB3, SERPINB4, SPRR2F, SPRR2E, SPRR2A, SPRR2D, KRT17, KRT16, KRT14, DSG3, KRT5, KRT6C, KRT6A, and KRT6B.

In some embodiments, a TME signature comprises a gene group score for the Neuroendocrine differentiation group. In some embodiments, this gene group score may be calculated using RNA expression levels of at least three genes (e.g., at least 3, at least 4, at least 5, at least 6, at least 7, at least 8, at least 9, at least 10, at least 11, or at least 12) in the Neuroendocrine differentiation group, which is defined by its constituent genes: PLEKHG4B, GNG4, PEG10, SOX2, TUBB2B, CHGB, SYP, ENO2, SV2A, MSI1, RND2, and APLP1.

In some embodiments, a TME signature comprises a gene group score for the FGFR3 co-expressed group. In some embodiments, this gene group score may be calculated using RNA expression levels of at least three genes (e.g., at least 3, at least 4, at least 5, at least 6, at least 7, at least 8, at least 9, at least 10, at least 11, at least 12, at least 13, at least 14, at least 15, or at least 16) in the FGFR3 co-expressed group, which is defined by its constituent genes: FGFR3, TP63, IRS1, WNT7B, CAPNS2, ZNF385A, SMAD3, SLC2A9, DUOXA1, SYTL1, SEMA4B, CLCA4, PLCH2, SSH3, PTPN13, and TMPRSS4.

In some embodiments, determining a UC TME signature comprises determining a respective gene group score for each of at least two of the following gene groups, using, for a particular gene group, RNA expression levels for at least three genes in the particular gene group to determine the gene group score for the particular group, the gene groups including: MHC type I group: HLA-C, TAPBP, HLA-B, B2M, TAP2, HLA-A, TAP1, NLRC5; MHC type II group: HLA-DQB1, HLA-DMA, HLA-DMB, HLA-DRA, CIITA, HLA-DQA1, HLA-DPB1, HLA-DRB1, HLA-DPA1; Coactivation molecules group: TNFRSF4, CD27, CD80, CD40LG, TNFRSF9, CD40, CD28, ICOSLG, CD83, TNFSF9, CD70, TNFSF4, ICOS, CD86; Effector cells group: ZAP70, GZMB, GZMK, IFNG, FASLG, EOMES, TBX21, GZMA, CD8A, GNLY, PRF1, CD8B; Natural killer cells group: NKG7, FGFBP2, CD244, KLRK1, KIR2DL4, CD226, KLRF1, GNLY, GZMB, KLRC2, NCR1, GZMH, IFNG, SH2D1B, NCR3, EOMES, CD160; T cells group: TRBC2, CD3E, CD3G, ITK, CD28, TRBC1, TRAT1, TBX21, CD5, TRAC, CD3D; T-helper cells type 1 group: IL21, TBX21, IL12RB2, CD40LG, IFNG, IL2, STAT4; T-helper cells type 2 group: IL13, CCR4, IL10, IL4, IL5; B cells group: CD22, TNFRSF13C, STAP1, CD79B, PAX5, CR2, TNFRSF13B, CD79A, TNFRSF17, FCRL5, MS4A1, CD19, BLK; Macrophages group: MRC1, SIGLEC1, MSR1, CD163, CSF1R, CD68, IL4Il, IL10; Macrophages type 1 group: CMKLR1, SOCS3, IRF5, NOS2, IL1B, IL12B, IL23A, TNF, IL12A; Antitumor cytokines group: CCL3, IL21, IFNB1, IFNA2, TNF, TNFSF10; Checkpoint inhibition group: PDCD1, BTLA, HAVCR2, CD274, VSIR, LAG3, TIGIT, PDCD1LG2, CTLA4; T-regulatory cells group: IKZF2, TNFRSF18, IL10, FOXP3, CCR8, IKZF4, CTLA4; Neutrophils group: CD177, FFAR2, PGLYRP1, CXCR1, MPO, CXCR2, ELANE, CTSG, PRTN3, FCGR3B; MDSC group: ARG1, IL6, CYBB, IL10, PTGS2, IDO1, IL4I1; Protumor cytokines group: TGFB2, MIF, IL6, TGFB3, TGFB1, IL22, IL10; Cancer associated fibroblasts (CAF) group: COL6A3, PDGFRB, COL6A1, MFAP5, COL5A1, FAP, PDGFRA, FGF2, ACTA2, COL6A2, FBLN1, CD248, COL1A1, MMP2, COL1A2, MMP3, LUM, CXCL12, LRP1; Matrix group: LAMC2, TNC, COL11A1, VTN, LAMB3, COL1A1, FN1, LAMA3, LGALS9, COL1A2, COL4A1, COL5A1, ELN, LGALS7, COL3A1; Matrix remodeling group: ADAMTS4, ADAMTS5, CA9, LOX, MMP1, MMP11, MMP12, MMP2, MMP3, MMP7, MMP9, PLOD2; Angiogenesis group: VEGFC, VEGFA, PDGFC, KDR, CDH5, VEGFB, PGF, TEK, ANGPT2, CXCR2, FLT1, CXCL8, VWF, ANGPT1, CXCL5; Endothelium group: KDR, CDH5, NOS3, VCAM1, VWF, FLT1, MMRN1, CLEC14A, ENG, MMRN2; Proliferation rate group: CCND1, CCNB1, CETN3, CDK2, E2F1, AURKA, BUB1, AURKB, PLK1, MCM6, ESCO2, MYBL2, MKI67, MCM2, CCNE1; and Epithelial to mesenchymal transition group: CDH2, ZEB1, ZEB2, TWIST1, SNAI1, SNAI2, TWIST2; Luminal differentiation group: PWRN1, PWRN3, GSTM5, GSTM4, GSTM2, ZNF321P, ZNF320, ZNF66, ZNF737, KRT20, UPK1B, FOXA1, ACER2, SEMA5A, PPARG, GATA3, SNX31, UPK2, UPK1A; Basal differentiation group: TM4SF19, SERPINB13, SERPINB3, SERPINB4, SPRR2F, SPRR2E, SPRR2A, SPRR2D, KRT17, KRT16, KRT14, DSG3, KRT5, KRT6C, KRT6A, KRT6B; Neuroendocrine differentiation group: PLEKHG4B, GNG4, PEG10, SOX2, TUBB2B, CHGB, SYP, ENO2, SV2A, MSI1, RND2, APLP1; and FGFR3 co-expressed group: FGFR3, TP63, IRS1, WNT7B, CAPNS2, ZNF385A, SMAD3, SLC2A9, DUOXA1, SYTL1, SEMA4B, CLCA4, PLCH2, SSH3, PTPN13, TMPRSS4. Lists of gene groups are provided in Table 1.

As described above, aspects of the disclosure relate to determining a urothelial cancer TME signature for a subject. That signature may include gene group scores (e.g., gene group scores generated using RNA expression data for gene groups listed in Table 1). Aspects of determining of TME signatures is described next with reference to FIG. 3.

In some embodiments, a TME signature comprises gene group scores generated using a gene set enrichment analysis (GSEA) technique to determine a gene group score for one or more (e.g., 1, 2, 3, 4, 5, 6, 7, 8, 9, 10, 11, 12, 13, 14, 15, 16, 17, 18, 19, 20, 21, 22, 23, 24, 25, 26, 27, or 28) gene groups listed in Table 1. In some embodiments, a TME signature comprises gene group scores generated using a gene set enrichment analysis (GSEA) technique to determine a gene group score for three or more (e.g., 3, 4, 5, 6, 7, 8, 9, 10, 11, 12, 13, 14, 15, 16, 17, 18, 19, 20, 21, 22, 23, 24, 25, 26, 27, or 28) gene groups listed in Table 1.

In some embodiments, each gene group score is generated using a gene set enrichment analysis (GSEA) technique, using RNA expression levels of at least some genes in the gene group. In some embodiments, using a GSEA technique comprises using single-sample GSEA. Aspects of single sample GSEA (ssGSEA) are described in Barbie et al. Nature. 2009 Nov 5; 462(7269): 108-112, the entire contents of which are incorporated by reference herein. In some embodiments, ssGSEA is performed according to the following formula:

$$s s G S E A s c o r e = \frac{{\sum }_{i}^{N} {r}_{i}^{1.25}}{{\sum }_{i}^{N} {r}_{i}^{0.25}} - \frac{\left(M - N + 1\right)}{2}$$

where rᵢ represents the rank of the ith gene in expression matrix, where N represents the number of genes in the gene set (e.g., the number of genes in the first gene group when ssGSEA is being used to determine a gene group score for the first gene group using expression levels of the genes in the first gene group), and where M represents total number of genes in expression matrix. Additional, suitable techniques of performing GSEA are known in the art and are contemplated for use in the methods described herein without limitation. In some embodiments, a TME signature is calculated by performing ssGSEA on expression data from a plurality of subjects, for example expression data from one or more cohorts of subjects, such as GSE124305, GSE87304, GSE128959, GSE83586, GSE70691, GSE48075, GSE13507, GSE69795, GSE32894, GSE154261, GSE133624, and TGCA-BLCA, etc., in order to produce a plurality of enrichment scores.

FIG. 3 depicts an illustrative example of how gene group scores may be determined as part of act 108 of process 100. As shown in the example of FIG. 3, a “TME signature” comprises multiple gene group scores 320 determined for respective multiple gene groups. Each gene group score, for a particular gene group, is computed by performing GSEA 310 (e.g., using ssGSEA) on RNA expression data for one or more (e.g., at least two, at least three, at least four, at least five, at least six, etc., or all) genes in the particular gene group 300.

For example, as shown in FIG. 3, a gene group score (labelled “Gene Group Score 1”) for gene group 1 (e.g., the T reg group) is computed from RNA expression data for one or more genes in gene group 1. As another example, a gene group score (labelled “Gene Group Score 2”) for gene group 2 (e.g., the T cells group) is computed from RNA expression data for one or more genes in gene group 2. As another example, a gene group score (labelled “Gene Group Score 3”) for gene group 3 (e.g., the NK cells group) is computed from RNA expression data for one or more genes in gene group 3. As another example, a gene group score (labelled “Gene Group Score 4”) for gene group 4 (e.g., the B cells group) is computed from RNA expression data for one or more genes in gene group 4. As another example, a gene group score (labelled “Gene Group Score 5”) for gene group 5 (e.g., the MDSC group) is computed from RNA expression data for one or more genes in gene group 5. As another example, a gene group score (labelled “Gene Group Score 6”) for gene group 6 (e.g., the CAF group) is computed from RNA expression data for one or more genes in gene group 6. As another example, a gene group score (labelled “Gene Group Score 7”) for gene group 7 (e.g., the Proliferation rate group) is computed from RNA expression data for one or more genes in gene group 7. As another example, a gene group score (labelled “Gene Group Score 8”) for gene group 8 (e.g., the coactivation molecules group) is computed from RNA expression data for one or more genes in gene group 8.

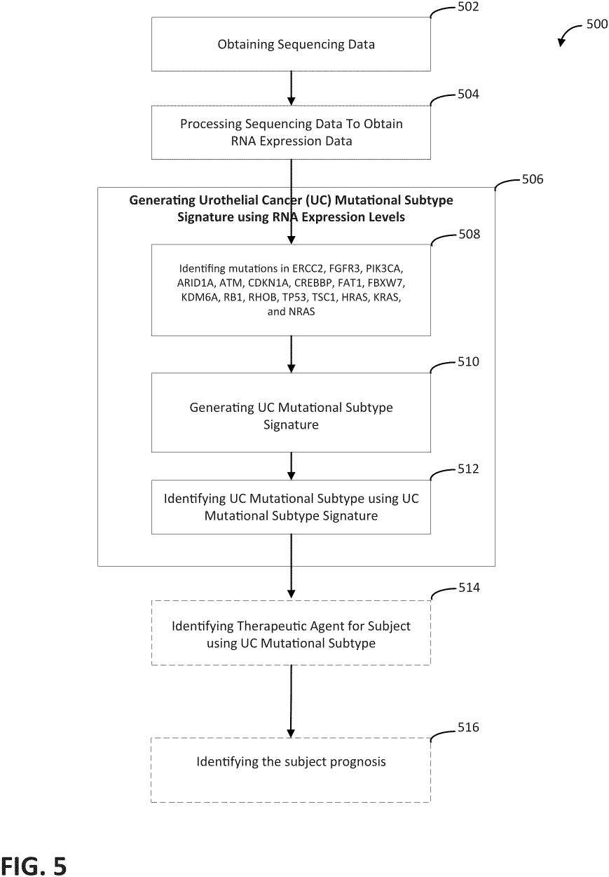

Although the example of FIG. 3 shows that the TME signature includes eight gene group scores for a respective set of eight gene groups, it should be appreciated that in other embodiments, the TME signature may include scores for any suitable number of gene groups (e.g., not just 8; the number of groups could be fewer or greater than 8). As indicated by the vertical ellipsis in FIG. 5, determining gene group scores of a TME signature may comprise determining gene group scores for 9, 10, 11, 12, 13, 14, 15, 16, 17, 18, 19, 20, or more gene groups using RNA expression data from one or more respective genes in each respective gene group, as aspects of the technology described herein are not limited in this respect. In another example, a TME signature may include scores for only a subset of the gene groups listed in Table 1. As another example, the gene group score may include one or more scores for one or more gene groups other than those gene groups listed in Table 1 (either in addition to the score(s) for the groups in Table 1 or instead of one or more of the scores for the groups in Table 1).

In some embodiments, RNA expression levels for a particular gene group may be embodied in at least one data structure having fields storing the expression levels. The data structure or data structures may be provided as input to software comprising code that implements a GSEA technique (e.g., the ssGSEA technique) and processes the expression levels in the at least one data structure to compute a score for the particular gene group.

The number of genes in a gene group used to determine a gene group score may vary. In some embodiments, all RNA expression levels for all genes in a particular gene group may be used to determine a gene group score for the particular gene group. In other embodiments, RNA expression data for fewer than all genes may be used (e.g., RNA expression levels for at least two genes, at least three genes, at least five genes, between 2 and 10 genes, between 5 and 15 genes, or any other suitable range within these ranges).

In some embodiments, RNA expression levels for a particular gene group may be embodied in at least one data structure having fields storing the expression levels. The data structure or data structures may be provided as input to software comprising code that is configured to perform suitable scaling (e.g., median scaling) to produce a score for the particular gene group.

In some embodiments, ssGSEA is performed on expression data comprising three or more (e.g., 3, 4, 5, 6, 7, 8, 9, 10, 11, 12, 13, 14, 15, 16, 17, 18, 19, or 20) gene groups set forth in Table 1. In some embodiments, each of the gene groups separately comprises one or more (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, or more) genes listed in Table 1. In some embodiments, a TME signature is produced by performing ssGSEA on all of the gene groups in Table 1, each gene group including all listed genes in Table 1. In some embodiments, one or more (e.g., a plurality) of gene group scores are normalized in order to produce a TME signature for the expression data (e.g., expression data of the subject or of a cohort of subjects). In some embodiments, the gene group scores are normalized by median scaling. In some embodiments, the gene group scores are normalized by rank estimation and median scaling. In some embodiments, median scaling comprises clipping the range of gene group scores, for example clipping to about -1.0 to about + 1.0, -2.0 to about +3.0, -3.0 to about +3.0, -4.0 to +4.0, -5.0 to about +5.0. In some embodiments, median scaling produces a TME signature of the subject.

In some embodiments, a TME signature of a subject processed using a clustering algorithm to identify a tumor microenvironment type (e.g. a UC TME type). In some embodiments, the clustering comprises unsupervised clustering. In some embodiments, the unsupervised clustering comprises a dense clustering approach. In some embodiments, the unsupervised clustering comprises a hierarchical clustering approach. In some embodiments, clustering comprises calculating intersample similarity (e.g., using a Pearson correlation coefficient that, for example, may take on values in the range of [-1,1]), converting the distance matrix into a graph where each sample forms a node and two nodes form an edge with a weight equal to their Pearson correlation coefficient, removing edges with weight lower than a specified threshold, and applying a Louvain community detection algorithm to calculate graph partitioning into clusters. In some embodiments, the optimum weight threshold for observed clusters was calculated by employing minimum DaviesBouldin, maximum Calinski-Harabasz, and Silhouette techniques. In some embodiments, separations with low-populated clusters (< 5% of samples) are excluded.

In some embodiments, a TME signature of a subject is compared to pre-existing clusters of TME types and assigned a TME type based on that comparison.

Some aspects of determining gene group scores for gene groups are also described in U.S. Pat. Publication No. 2020-0273543, entitled “SYSTEMS AND METHODS FOR GENERATING, VISUALIZING AND CLASSIFYING MOLECULAR FUNCTIONAL PROFILES”, the entire contents of which are incorporated by reference herein.

### Generating TME Signature and Identifying TME Type

As described herein, FIGS. 1-3 illustrate the determination of a subject’s urothelial cancer TME signature, identification of the subject’s TME type using the TME signature, and identification of whether the subject is likely to respond to a therapy based on the identified TME type.

As described herein, in some embodiments, one of a plurality of different urothelial cancer TME types may be identified for the subject using the TME signature determined for the subject using the techniques described herein. In some embodiments, the plurality of UC TME types comprises an Immune Desert (D) type, Immune Enriched (IE) type, Fibrotic (F) type, Immune Enriched -Fibrotic (IE/F) type, Immune Desert type, FGFR-altered (D/FGFR) type, Fibrotic - Basal (Bas) type, and Neuroendocrine-like (NE) type, as described herein and further below.

In some embodiments, each of the plurality of TME types is associated with a respective TME signature cluster in a plurality of TME signature clusters. The TME type for a subject may be determined by: (1) associating the TME signature of the subject with a particular one of the plurality of TME signature clusters; and (2) identifying the TME type for the subject as the TME type corresponding to the particular one of the plurality of TME signature clusters to which the TME signature of the subject is associated.

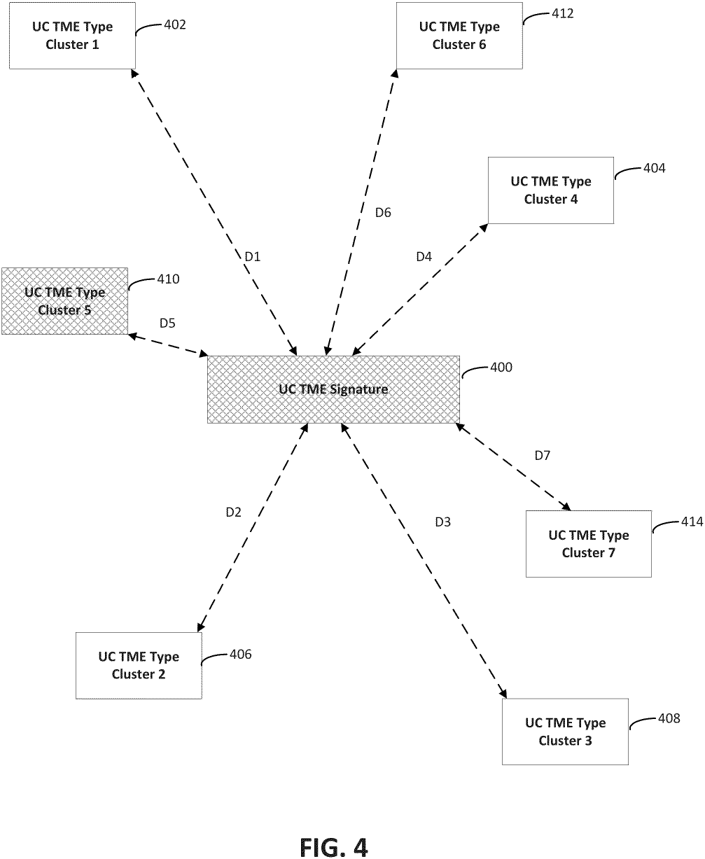

FIG. 4 shows an illustrative UC TME signature 400. In some embodiments, the TME signature (e.g., UC TME signature) comprises at least three gene group scores for gene groups listed in Table 1. However, it should be appreciated, that a TME signature may include fewer scores than the number of scores shown in FIG. 3 (e.g., by omitting scores for one or more of the gene groups listed in Table 1) or more scores than the number of scores shown in FIG. 3 (e.g., by including scores for one or more other gene groups in addition to or instead of the gene groups listed in Table 1). In some embodiments, a TME signature may be embodied in at least one data structure comprising fields storing the gene group scores part of the TME signature.

In some embodiments, the TME signature clusters may be generated by: (1) obtaining TME signatures (using the techniques described herein) for a plurality of subjects; and (2) clustering the TME signatures so obtained into the plurality of clusters. Any suitable clustering technique may be used for this purpose including, but not limited to, a dense clustering algorithm, spectral clustering algorithm, k-means clustering algorithm, hierarchical clustering algorithm, and/or an agglomerative clustering algorithm.

For example, intersample similarity may be calculated using a Pearson correlation. A distance matrix may be converted into a graph where each sample forms a node and two nodes form an edge with a weight equal to their Pearson correlation coefficient. Edges with weight lower than a specified threshold may be removed. A Louvain community detection algorithm may be applied to calculate graph partitioning into clusters. To mathematically determine the optimum weight threshold for observed clusters minimum DaviesBouldin, maximum Calinski-Harabasz, and Silhouette techniques may be employed. Separations with low-populated clusters (< 5% of samples) may be excluded.

Accordingly, in some embodiments, generating the TME signature clusters involves: (A) obtaining multiple sets of RNA expression data obtained by sequencing biological samples from multiple respective subjects, each of the multiple sets of RNA expression data indicating RNA expression levels for genes in a first plurality of gene groups (e.g., one or more of the gene groups in Table 1); (B) generating multiple TME signatures from the multiple sets of RNA expression data, each of the multiple TME signatures comprising gene group scores for respective gene groups, the generating comprising, for each particular one of the multiple TME signatures: (i) determining the TME signature by determining the gene group scores using the RNA expression levels in the particular set of RNA expression data for which the particular one TME signature is being generated, and (ii) clustering the multiple signatures to obtain the plurality of TME signature clusters.

The resulting TME signature clusters may each contain any suitable number of TME signatures (e.g., at least 10, at least 100, at least 500, at least 500, at least 1000, at least 5000, between 100 and 10,000, between 500 and 20,000, or any other suitable range within these ranges), as aspects of the technology described herein are not limited in this respect.

The number of TME signature clusters in this example is seven. And although, in some embodiments, it may be possible that the number of clusters is different, it should be appreciated that an important aspect of the present disclosure is the inventors’ discovery that urothelial cancer may be characterized into seven TME types based upon the generation of TME signatures using methods described herein.

For example, as shown in FIG. 4, a subject’s UC TME signature 400 may be associated with one of seven UC TME clusters: 402, 404, 406, 408, 410, 412, and 414. Each of the clusters 402, 404, 406, 408, 410, 412, and 414 may be associated with respective UC TME type. In this example, the UC TME signature 400 is compared to each cluster (e.g., using a distance-based comparison or any other suitable metric) and, based on the result of the comparison, the UC TME signature 400 is associated with the closest signature cluster (when a distance-based comparison is performed, or the “closest” in the sense of whatever metric or measure of distance is used). In this example, UC TME signature 400 is associated with UC TME Type Cluster 5 410 (as shown by the consistent shading) because the measure of distance D5 between the UC TME signature 400 and (e.g., a centroid or other point representative of) cluster 410 is smaller than the measures of the distance D1, D2, D3,D4, D6, and D7 between the UC TME signature 400 and (e.g., a centroid or other point(s) representative of) clusters 402, 404, 406, 408, 412, and 414, respectively.

In some embodiments, a subject’s TME signature may be associated with one of seven urothelial cancer TME signature clusters by using a machine learning technique (e.g., such as k-nearest neighbors (KNN) or any other suitable classifier) to assign the TME signature to one of the seven urothelial cancer TME signature clusters. The machine learning technique may be trained to assign TME signatures on the meta-cohorts represented by the signatures in the clusters.

In some embodiments, UC TME types comprise an Immune Desert (D) type, Immune Enriched (IE) type, Fibrotic (F) type, Immune Enriched -Fibrotic (IE/F) type, Immune Desert type, FGFR-altered (D/FGFR) type, Fibrotic - Basal (Bas) type, and Neuroendocrine-like (NE) type. The urothelial cancer TME types described herein may be described by qualitative characteristics, for example high signals for certain gene expression signatures or scores or low signals for certain other gene expression signatures or scores. In some embodiments, a “high” signal refers to a gene expression signal or score (e.g., an enrichment score) that is at least 1-fold, 2-fold, 3-fold, 4-fold, 5-fold, 6-fold, 7-fold, 8-fold, 9-fold, 10-fold, 20-fold, 50-fold, 100-fold, 1000-fold, or more increased relative to the score of the same gene or gene group in a subject having a different type of urothelial cancer (e.g., a different TME type within the same type cancer, for example urothelial cancer). In some embodiments, a “low” signal refers to a gene expression signal or score (e.g., an enrichment score,) that is at least 1-fold, 2-fold, 3-fold, 4-fold, 5-fold, 6-fold, 7-fold, 8-fold, 9-fold, 10-fold, 20-fold, 50-fold, 100-fold, 1000-fold, or more decreased relative to the score of the same gene or gene group in a subject having a different type of TME (e.g., a different TME type within the same type of cancer, for example urothelial cancer).

The tumor microenvironment of UC may contain variable numbers of immune cells, stromal cells, blood vessels and extracellular matrix.

In some embodiments, UC TME type Immune Desert, FGFR-altered is characterized by a “desert” TME with an increased proportion of malignant cells with active signature of luminal differentiation, high frequency of FGFR3 mutations, CDKN2A deletions, and high FGFR3 expression relative to other UC TME types. In some embodiments, this UC TME type has low immune infiltration, and patients have a moderate level of ICI response. The UCs of this type predominantly have a papillary phenotype, and the lowest tumor stage and grade relative to other UC TME types. In some embodiments, Desert, FGFR-altered UC TME type is characterized by a hyperactivated FGFR3 axis. In some embodiments, Desert, FGFR-altered UC TME type patients are suitable targets for anti-FGFR therapy, for example erdafitinib.

In some embodiments, Immune Desert UC TME type is characterized by a “desert” TME with malignant cells that show active signature of luminal differentiation, higher genomic instability, frequent mutations in TP53 and RB1, MCL1 amplifications, RB1 deletions, high expression of ERBB2 and APOBEC3B and high proliferation relative to other UC TME types. In some embodiments, subjects of this UC TME type have a moderate rate of ICI response. ERBB2 is a potential target for therapy in patients having this UC TME type. A large number of genomic rearrangements present in this UC TME type are also targets for PARP inhibitors. In some embodiments, Desert type patients are suitable targets for ERBB2-targeting therapy or PARP inhibitors.

In some embodiments, Immune Enriched UC TME type is characterized by an “anti-tumor immunity” TME enriched for T-, B- and NK-cells. Malignant cells present an active signature of luminal differentiation, high frequency of ARID1B mutations, MCL1 amplifications, and high expression of PD1. In some embodiments, patients with this UC TME type have the highest ICI response rate and the best overall survival (OS) rate relative to other UC TME types. In some embodiments, Immune Enriched UC TME type patients are suitable targets for ICI therapies, for example PD-1 inhibitors, PD-L1 inhibitors, or CTLA-4 inhibitors.

In some embodiments, Fibrotic UC TME type is characterized by a “mesenchymal” TME enriched for cancer-associated fibroblasts (CAFs), angiogenesis, endothelium and protumor cytokines. Malignant cells show a high rate of TNFRSF14 deletions, activation of the TGFB signaling and epithelial-to-mesenchymal transition (EMT) relative to other UC TME types. In some embodiments, Fibrotic UC TME type patients have the lowest proportion of malignant cells relative to other UC TME types. In some embodiments, Fibrotic UC TME type patients are characterized by high activity of stromal components and the TGFb pathway, and may be candidates for the TGFb-inhibitors, which can change the tumor microenvironment (TME) from pro-tumor to anti-tumor. This UC TME type is also characterized by low activity of DNA damage repair genes, or mutations in these genes, in particular in BRCA1, and can be targeted by PARP inhibitors. In some embodiments, Fibrotic UC TME type patients are suitable targets for TGFb-inhibitors or PARP inhibitors.

In some embodiments, Immune Enriched, UC TME type is characterized by a “mixed” TME enriched for angiogenesis, macrophages, MDSC, T- and NK-cells. Malignant cells present an active signature of basal differentiation, a high frequency of RB1 and EP300 mutations, and activation of NFkB and JAK-STAT pathways, relative to other UC TME types. The UCs of this TME type are prone to invasion. Patients show a high response rate and overall survival (OS) in the context of ICI therapy. In some embodiments, Immune Enriched, Fibrotic UC TME type patients are suitable targets for ICI therapies.

In some embodiments, Fibrotic, Basal UC TME type (also referred to as “Basal”) is characterized by a “mesenchymal” TME enriched for cancer-associated fibroblasts (CAFs) and extracellular matrix (ECM) components. Malignant cells show the highest activity of basal differentiation signature relative to other UC TME types, and activation of hypoxia and matrix remodeling pathways. Basal UC TME patients have the worst ICI response rate and worst prognosis for cisplatin-based and ICI therapies of all UC TME types described herein. In some embodiments, the Fibrotic, Basal UC TME type is characterized by very high-risk of disease progression and treatment resistance. In some embodiments, Fibrotic, Basal UC TME type patients are suitable targets for aggressive treatments, including radiotherapy and chemotherapy, early in the course of disease.

In some embodiments, Neuroendocrine-like UC TME type is characterized by a “desert” TME with high proportion of malignant cells with active signature of neuroendocrine differentiation, and tendency to have a high rate of TP53 and RB1 mutations relative to other UC TME types. The UCs of this TME type show a tendency to invasion, non-papillary histology, high tumor stage and low grade. NE-like UC TME patients have the worst OS on cisplatin-based therapy, but the best outcome on ICI therapy relative to other UC TME types. In some embodiments, NE-like UC TME subjects have the best overall survival (OS) for atezolizumab treatment of all UC TME types. In some embodiments, Neuroendocrine-like type patients are suitable targets for ICI therapies, such as atezolizumab.

Tables 2-4 below describe examples of urothelial cancer TME signatures and gene group scores produced by ssGSEA analysis and normalization (e.g., median scaling) of expression data from one or more urothelial cancer subjects.

TABLE 2

Representative gene group score values for UC TME types- 25^{th} percentile.

| Gene Group | Fibrotic | Fibrotic, Basal | Immune Desert | Immune Desert, FGFR-Altered | Immune-Enriched, Fibrotic | Immune-Enriched, Non′-fibrotic | Neuroen docrine-like |
|---|---|---|---|---|---|---|---|
| MHCI | -0.552862 | 0.069567 | -0.892747 | -1.117996 | 0.595351 | 0.122938 | -1.714654 |
| MHCII | -0.105692 | -0.574391 | -1.113298 | -1.581801 | 0.467142 | 0.357506 | -1.974138 |
| Coactivation molecules | -0.223266 | -0.261255 | -0.995294 | -1.038883 | 0.518279 | 0.470903 | -1.304764 |
| Effector cells | -0.365011 | -0.248988 | -0.843043 | -0.935191 | 1.027803 | 0.643198 | -0.786903 |
| NK cells | -0.490589 | -0.279863 | -0.853303 | -0.904665 | 0.837922 | 0.668967 | -0.919352 |
| T cells | -0.272846 | -0.48294 | -0.954172 | -0.912639 | 0.506964 | 0.770252 | -1.343557 |
| B cells | -0.344703 | -0.609432 | -0.666422 | -0.819023 | -0.191205 | 0.781986 | -0.645564 |
| M1 signatures | -0.532483 | -0.323009 | -0.906751 | -0.965925 | 0.136898 | -0.108055 | -1.78195 |
| Th1 signature | -0.510765 | -0.122792 | -0.865176 | -0.896308 | 0.615597 | 0.429284 | -1.074794 |
| Antitumor cytokines | -0.654401 | 0.241184 | -1.049644 | -0.965912 | 0.653189 | -0.09576 | -1.827309 |
| Checkpoint inhibition | -0.273643 | -0.16761 | -0.93474 | -0.975165 | 1.093387 | 0.446828 | -1.237018 |
| Treg | -0.6862 | -0.722741 | -0.702991 | -0.785194 | -0.651497 | 0.352406 | -2.297901 |
| Neutrophil signature | -0.093376 | -0.349252 | -0.895979 | -0.846463 | -0.281018 | -0.342287 | -1.297896 |
| MDSC | -0.105459 | -0.022302 | -1.06322 | -1.065932 | 0.748467 | -0.087638 | -1.616704 |
| Macrophages | 0.059671 | -0.193313 | -0.975728 | -1.092276 | 0.770628 | 0.067464 | -1.313158 |
| Th2 signature | -0.400223 | -0.577306 | -0.915059 | -0.772018 | -0.186603 | -0.13085 | -1.288389 |
| Protumor cytokines | 0.330982 | 0.026926 | -1.204881 | -0.76479 | 0.377978 | -0.629616 | -1.217306 |
| CAF | 0.628469 | -0.224284 | -1.171957 | -1.117209 | -0.165467 | -0.544925 | -1.675461 |
| Matrix | 0.147787 | 0.515038 | -1.243248 | -0.840199 | 0.137198 | -0.78148 | -1.175865 |
| Matrix remodeling | -0.081517 | 0.457237 | -1.054805 | -0.887943 | 0.460813 | -0.66822 | -0.947066 |
| Angiogenesis | 0.221564 | -0.034406 | -1.132473 | -0.804286 | -0.14041 | -0.985928 | -1.342917 |
| Endothelium | 0.583161 | -1.175352 | -1.015395 | -0.592961 | -0.524 | -0.338208 | -2.164902 |
| Proliferation rate | -1.34903 | -0.168569 | 0.209503 | -1.199934 | -0.112684 | -0.923673 | -1.151458 |
| EMT signature | 0.559356 | -0.032445 | -1.357514 | -0.797692 | 0.156422 | -0.636178 | -0.105192 |
| Luminal | -0.943263 | -2.065646 | 0.016378 | 0.110416 | -2.102315 | -0.591775 | -2.268381 |
| Basal | -0.652625 | 1.306993 | -0.855232 | -0.50585 | -0.130424 | -0.654474 | -1.176254 |
| FGFR3 co-expressed genes | -1.137634 | -0.21699 | -1.353072 | 0.3374 | -1.64406 | -0.879529 | -2.450807 |
| Neuroendocrine | -0.464592 | -0.439172 | 0.211258 | -1.056461 | -0.656325 | -0.633114 | 3.314999 |

TABLE 3

Representative gene group score values for UC TME types- 50^{th} percentile.

| Gene Group | Fibrotic | Fibrotic, Basal | Immune Desert | Immune Desert, FGFR-Altered | Immune-Enriched, Fibrotic | Immune-Enriched, Non-fibrotic | Neuroe ndocri ne-like |
|---|---|---|---|---|---|---|---|
| MHCI | -0.142125 | 0.555226 | -0.342055 | -0.665326 | 0.990077 | 0.65983 | - 0.77003 9 |
| MHCII | 0.29145 | 0.008338 | -0.407503 | -0.850955 | 0.763798 | 0.690522 | - 1.21878 5 |
| Coactivation_mo lecules | 0.272217 | 0.159599 | -0.46925 | -0.639088 | 1.131998 | 0.990134 | - 0.93801 9 |
| Effector cells | 0.083957 | 0.245337 | -0.4519 | -0.566423 | 1.676015 | 1.171291 | - 0.46019 3 |
| NK cells | 0.054807 | 0.227139 | -0.424062 | -0.54011 | 1.487083 | 1.126682 | - 0.37183 7 |
| T cells | 0.241325 | -0.03202 | -0.492149 | -0.504337 | 1.1095 | 1.268067 | - 1.00777 7 |
| B cells | 0.411027 | -0.125639 | -0.215483 | -0.459366 | 0.30386 | 1.618321 | - 0.31463 4 |
| M1 signatures | 0.191015 | 0.264975 | -0.342009 | -0.449028 | 0.788906 | 0.433736 | - 1.31001 8 |
| Th1_signature | -0.005436 | 0.343068 | -0.498201 | -0.454153 | 1.414314 | 1.043331 | - 0.49637 7 |
| Antitumor cytokines | -0.111114 | 0.719589 | -0.450954 | -0.489226 | 1.064282 | 0.388793 | - 1.43192 8 |
| Checkpoint inhibition | 0.116217 | 0.291348 | -0.540982 | -0.609762 | 1.727417 | 0.988483 | - 0.65572 3 |
| Treg | -0.120422 | -0.025415 | -0.242265 | -0.300407 | 0.217925 | 0.910613 | - 1.97045 8 |
| Neutrophil signature | 0.720525 | 0.22941 | -0.450811 | -0.31695 | 0.527465 | 0.22073 | - 0.74637 3 |
| MDSC | 0.376669 | 0.476399 | -0.639986 | -0.619203 | 1.341167 | 0.411089 | - 0.76272 5 |
| Macrophages | 0.558456 | 0.201523 | -0.601355 | -0.676549 | 1.199628 | 0.533567 | - 0.87691 8 |
| Th2 signature | 0.26745 | -0.08526 | -0.376776 | -0.222015 | 0.457455 | 0.613326 | - 0.75243 6 |
| Protumor cytokines | 0.968856 | 0.563373 | -0.768338 | -0.391824 | 0.97007 | -0.124211 | - 0.47504 9 |
| CAF | 0.971123 | 0.383513 | -0.586564 | -0.541458 | 0.402546 | 0.026354 | - 0.99414 5 |
| Matrix | 0.581967 | 0.966377 | -0.735214 | -0.343104 | 0.798251 | -0.25881 | - 0.70738 4 |
| Matrix remodeling | 0.526767 | 1.029001 | -0.582347 | -0.434684 | 0.875161 | -0.236937 | - 0.61468 2 |
| Angiogenesis | 0.767313 | 0.483528 | -0.596837 | -0.245766 | 0.506551 | -0.366247 | - 0.71190 2 |
| Endothelium | 1.114367 | -0.518015 | -0.457124 | -0.078934 | 0.058112 | 0.169504 | - 1.42148 |
| Proliferation rate | -0.814462 | 0.426058 | 0.583073 | -0.49101 | 0.419932 | -0.249352 | - 1.28888 |
| EMT signature | 1.012582 | 0.362825 | -0.866766 | -0.402099 | 0.668983 | -0.154021 | - 0.52412 7 |
| Luminal | -0.185071 | -1.39697 | 0.453011 | 0.481518 | -1.500862 | 0.063207 | - 1.92613 8 |
| Basal | -0.221594 | 1.879019 | -0.544287 | -0.081648 | 0.620969 | -0.253559 | - 0.91065 1 |
| FGFR3 co-expressed genes | -0.448846 | 0.258771 | -0.625896 | 0.735213 | -0.932169 | -0.198318 | - 2.05587 9 |
| Neuroendocrine | 0.071217 | 0.231089 | 0.85018 | -0.53571 | -0.009183 | -0.030415 | 4 |

TABLE 4

Representative gene group score values for UC TME types- 75^{th} percentile

| Gene Group | Fibrotic | Fibrotic, Basal | Immune Desert | Immune Desert, FGFR-Altered | Immune-Enriched, Fibrotic | Immune-Enriched, Non-fibrotic | Neuroendocrine-like |
|---|---|---|---|---|---|---|---|
| MHCI | 0.31534 | 0.947403 | 0.146367 | -0.12023 | 1.249494 | 1.073812 | -0.173948 |
| MHCII | 0.669703 | 0.439868 | 0.107651 | - 0.264851 | 1.047782 | 1.063371 | -0.460124 |
| Coactivation molecules | 0.866952 | 0.634955 | -0.045877 | - 0.170461 | 1.795457 | 1.668665 | -0.424567 |
| Effector cells | 0.674454 | 0.763555 | -0.085723 | - | 2.320957 | 1.853648 | -0.21814 |
| | | | | 0.152386 | | | |
| NK cells | 0.605201 | 0.771988 | -0.099245 | -0.11975 | 2.256992 | 1.762082 | 0.024039 |
| T cells | 0.720516 | 0.41569 | -0.050514 | 0.016608 | 1.787902 | 1.882693 | -0.383368 |
| B cells | 1.536794 | 0.492004 | 0.294924 | 0.015024 | 1.09659 | 2.595049 | 0.099288 |
| M1 signatures | 0.974053 | 0.978705 | 0.251432 | 0.132794 | 1.55655 | 1.059034 | -0.45355 |
| Th1 signature | 0.649177 | 1.045353 | -0.044802 | 0.067635 | 2.137028 | 1.730455 | -0.015935 |
| Antitumor cytokines | 0.429406 | 1.173762 | 0.017429 | 0.023086 | 1.55781 | 0.865672 | -0.912279 |
| Checkpoint inhibition | 0.668712 | 0.753041 | -0.091837 | 0.224873 | 2.382227 | 1.646407 | -0.17413 |
| Treg | 0.482074 | 0.662535 | 0.415097 | 0.339713 | 0.993348 | 1.481425 | -0.997932 |
| Neutrophil signature | 1.627153 | 0.826727 | 0.146512 | 0.2154 | 1.207568 | 0.966709 | -0.370816 |
| MDSC | 0.958493 | 0.954816 | -0.156055 | 0.150689 | 1.727184 | 1.026326 | -0.239773 |
| Macrophages | 1.09067 | 0.692219 | -0.120632 | 0.317175 | 1.827791 | 0.986629 | -0.489378 |
| Th2 signature | 1.017557 | 0.471701 | 0.269157 | 0.459688 | 1.072233 | 1.41926 | -0.492989 |
| Protumor cytokines | 1.719354 | 1.094608 | -0.217368 | 0.016043 | 1.409053 | 0.473738 | 0.35332 |
| CAF | 1.368355 | 0.818819 | -0.057161 | 0.00288 | 0.877489 | 0.551865 | -0.251934 |
| Matrix | 0.981858 | 1.439683 | -0.274107 | 0.081148 | 1.185565 | 0.233368 | -0.182566 |
| Matrix remodeling | 1.070997 | 1.527993 | -0.030622 | 0.096017 | 1.343456 | 0.195807 | -0.005963 |
| Angiogenesis | 1.476163 | 1.016725 | -0.008193 | 0.300159 | 1.14472 | 0.137833 | 0.029293 |
| Endothelium | 1.762299 | 0.040889 | 0.096155 | 0.584934 | 0.573579 | 0.801043 | -0.599275 |
| Proliferation_rate | -0.087196 | 0.833973 | 0.92781 | 0.162662 | 0.862046 | 0.397232 | 1.442424 |
| EMT signature | 1.520251 | 0.883754 | -0.304695 | 0.044755 | 1.25164 | 0.368775 | 1.2171 |
| Luminal | 0.299832 | -0.807632 | 0.781482 | 0.760786 | -0.899397 | 0.473808 | -1.528539 |
| Basal | 0.348975 | 2.360386 | 0.006953 | 0.460281 | 1.365889 | 0.382425 | -0.491387 |
| FGFR3 co-expressed genes | 0.108734 | 0.641698 | -0.039224 | 1.00806 | -0.217202 | 0.309091 | -1.450174 |
| Neuroendocrine | 0.634428 | 0.765998 | 1.463436 | 0.039957 | 0.798454 | 0.639719 | 4 |

In some embodiments, the present disclosure provides methods for identifying a subject having, suspected of having, or at risk of having UC as having an increased likelihood of having a good prognosis (e.g., as measured by overall survival (OS) or progression-free survival (PFS). In some embodiments, the method comprises determining a UC TME type of the subject as described herein.

In some embodiments, the methods comprise identifying the subject as having a decreased risk of UC progression relative to other UC TME types. In some embodiments, “decreased risk of UC progression” may indicate better prognosis of UC or decreased likelihood of having advanced disease in a subject. In some embodiments, “decreased risk of UC progression” may indicate that the subject who has UC is expected to be more responsive to certain treatments. For instance, “decreased risk of UC progression” indicates that a subject is at least 10%, 20%, 30%, 40%, 50%, 60%, 70%, 80%, 90%, or 100% likely to experience a progression-free survival event (e.g., relapse, retreatment, or death) than another UC patient or population of UC patients (e.g., patients having UC, but not the same UC TME type as the subject).

In some embodiments, the methods further comprise identifying the subject as having an increased risk of UC progression relative to other UC TME types. In some embodiments, “increased risk of UC progression” may indicate less positive prognosis of UC or increased likelihood of having advanced disease in a subject. In some embodiments, “increased risk of BC progression” may indicate that the subject who has UC is expected to be less responsive or unresponsive to certain treatments and show less or no improvements of disease symptoms. For instance, “increased risk of UC progression” indicates that a subject is at least 10%, 20%, 30%, 40%, 50%, 60%, 70%, 80%, 90%, or 100% more likely to experience a progression-free survival event (e.g., relapse, retreatment, or death) than another UC patient or population of UC patients (e.g., patients having UC, but not the same UC TME type as the subject).

In some embodiments, the methods described herein comprise the use of at least one computer hardware processor to perform the determination.

In some embodiments, the present disclosure provides a method for providing a prognosis, predicting survival, or stratifying patient risk of a subject suspected of having, or at risk of having UC. In some embodiments, the method comprises determining a UC TME type of the subject as described herein.

### Updating TME Clusters Based on New Data

Techniques for generating urothelial cancer TME clusters are described herein. It should be appreciated that the TME clusters may be updated as additional TME signatures are computed for patients. In some embodiments, the TME signature of the subject is one of a threshold number TME signatures for a threshold number of subjects. In some embodiments, when the threshold number of TME signatures is generated the TME signature clusters are updated. For example, once a threshold number of new TME signatures are obtained (e.g., 1 new signature, 10 new signatures, 100 new signatures, 500 new signatures, any suitable threshold number of signatures in the range of 10-1,000 signatures), the new signatures may be combined with the TME signatures previously used to generate the TME clusters and the combined set of old and new TME signatures may be clustered again (e.g., using any of the clustering algorithms described herein or any other suitable clustering algorithm) to obtain an updated set of TME signature clusters.

In this way, data obtained from a future patient may be analyzed in a way that takes advantage of information learned from patients whose TME signature was computed prior to that of the future patient. In this sense, the machine learning techniques described herein (e.g., the unsupervised clustering machine learning techniques) are adaptive and learn with the accumulation of new patient data. This facilitates improved characterization of the TME type that future patients may have and may improve the selection of treatment for those patients.

### Urothelial Cancer Mutational Subtypes

Aspects of the disclosure relate to methods for determining a urothelial cancer (UC) mutational subtype of a subject having, suspected of having, or at risk of having a urothelial cancer, the method comprising using at least one computer hardware processor to perform obtaining RNA expression data for the subject, the RNA expression data indicating RNA expression levels for genes of the subject; generating a UC mutational subtype signature for the subject using the RNA expression data, the generating comprising analyzing the RNA expression data to identify the presence or absence of one or more mutations in the one or more of the following genes: ERCC2, FGFR3, PIK3CA, ARID1A, ATM, CDKN1A, CREBBP, FAT1, FBXW7, KDM6A, RB1, RHOB, TP53, TSC1 HRAS, KRAS, and NRAS; and identifying, using the UC mutational subtype signature and from among a plurality of UC mutational subtypes, a UC mutational subtype for the subject.

Turning to the figures, FIG. 5 provides a description of one example of a process for using a computer hardware processor to perform a method of identifying the urothelial cancer (UC) mutational subtype of a subject, according to some aspects of the invention 500. First, sequencing data is obtained 502. Methods of obtaining sequencing data are described throughout the specification including in the section entitled Sequencing Data and Gene Expression Data. Next, the sequencing data is processed to obtain gene expression data 504. Gene expression data is used to determine a urothelial cancer (UC) mutational subtype for the subject 506. In some embodiments, the determining comprises processing the gene expression data to identify one or more mutations in one or more of the following genes, ERCC2, FGFR3, PIK3CA, ARID1A, ATM, CDKN1A, CREBBP, FAT1, FBXW7, KDM6A, RB1, RHOB, TP53, TSC1 HRAS, KRAS, and NRAS, 508. In some embodiments, the mutations are identified by performing filtering on the gene expression data to identify the one or more mutations, thereby generating a UC Mutational Subtype signature, 510.

As described above, at act 510, the UC mutational subtype signature is generated. In some embodiments, the UC mutational subtype signature consists of only of identification of the presence or absence of one or more mutations in at least some of the following genes, ERCC2, FGFR3, PIK3CA, ARID1A, ATM, CDKN1A, CREBBP, FAT1, FBXW7, KDM6A, RB1, RHOB, TP53, TSC1 HRAS, KRAS, and NRAS. In some embodiments, the UC mutational subtype signature comprises identification of the presence or absence of one or more mutations in each of the following genes: ERCC2, FGFR3, PIK3CA, ARID1A, ATM, CDKN1A, CREBBP, FAT1, FBXW7, KDM6A, RB1, RHOB, TP53, TSC1 HRAS, KRAS, and NRAS. In other embodiments, the UC mutational subtype signature includes identification of one or more mutations in one or more other genes in addition to the genes listed in FIG. 5.

Next, process 500 proceeds to act 512, where a UC mutational subtype is identified for the subject using the UC mutational subtype signature generated at act 510. This may be done in any suitable way. For example, in some embodiments, the each of the possible UC mutational subtypes is associated with a respective plurality of UC mutational subtype signature clusters. In such embodiments, a UC mutational subtype for the subject may be identified by associating the UC mutational subtype signature of the subject with a particular one of the plurality of UC mutational subtype signature clusters; and identifying the UC mutational subtype for the subject as the UC mutational subtype corresponding to the particular one of the plurality of UC mutational subtype signature clusters to which the UC mutational subtype signature of the subject is associated. Examples of UC mutational subtypes are described herein.

As described above, a subject’s UC mutational subtype is identified at act 512. In some embodiments, the UC mutational subtype of a subject is identified to be one of the following UC mutational subtypes: TP53-altered type, KDM6A-altered type, FGFR3-altered type, ARID1A-altered type, and Hypermutated (“HM”) type.

In some embodiments, the TP53-altered UC mutational subtype is characterized by frequent mutations in TP53 and RB1 genes. TP53-altered subtype patients have a moderate rate of ICI response but a low overall survival (OS) rate relative to other UC mutational subtypes.

In some embodiments, the KDM6A-altered UC mutational subtype is characterized by frequent mutations in the KDM6A gene. KDM6A subtype patients have a relatively low rate of ICI response and low overall survival (OS) rate relative to other UC mutational subtypes.

In some embodiments, FGFR3-altered UC mutational subtype is characterized by frequent mutations in FGFR3 and PIK3CA genes. FGFR3-altered subtype patients are candidates for anti-FGFR3 therapy. They have a relatively low rate of ICI response and a low overall survival (OS) rate.

In some embodiments, ARID1A-altered UC mutational subtype is characterized by frequent mutations in the ARID1A gene. ARID1A subtype patients have a high overall survival (OS) rate on anti-PDL1 therapy and moderate OS rate on cisplatin-based therapy.

In some embodiments, Hypermutated UC mutational subtype is characterized by high mutational burden (more than 20 mutations per megabase). Patients with Hypermutated subtype have the highest overall survival (OS) rate and highest response to ICI therapy of the UC mutational subtypes described herein.

Table 5 below describes examples of urothelial cancer mutational subtype signature clusters.

Mutational Clusters, % of samples with mutations in particular gene

| | ARID1A | FGFR3 | HM | KDM6A | NoSMG | TP53 |
|---|---|---|---|---|---|---|
| Hugo Symbol | | % | % | % | % | % |
| ARID1A | 61.54 | 8.18 | 31.25 | 8.59 | 6.25 | 4.32 |
| FGFR3 | 1.92 | 33.64 | 0 | 10.94 | 0 | 1.85 |
| TP53 | 26.92 | 9.09 | 37.5 | 28.12 | 20.83 | 59.26 |
| KDM6A | 13.46 | 13.64 | 31.25 | 41.41 | 4.17 | 8.02 |
| ERCC2 | 3.85 | 0.91 | 31.25 | 18.75 | 0 | 2.47 |
| RB1 | 14.42 | 1.82 | 25 | 4.69 | 7.29 | 26.54 |
| PIK3CA | 14.42 | 27.27 | 50 | 10.16 | 1.04 | 12.35 |
| CDKN1A | 6.73 | 16.36 | 12.5 | 2.34 | 0 | 5.56 |
| ATM | 3.85 | 7.27 | 37.5 | 15.62 | 5.21 | 7.41 |
| FBXW7 | 10.58 | 1.82 | 18.75 | 9.38 | 1.04 | 1.85 |
| KMT2D | 15.38 | 20.91 | 56.25 | 14.84 | 10.42 | 22.84 |
| NCOR1 | 3.85 | 5.45 | 31.25 | 4.69 | 3.12 | 4.94 |
| HRAS | 3.85 | 0 | 12.5 | 7.03 | 0 | 1.23 |
| EP300 | 6.73 | 8.18 | 37.5 | 12.5 | 5.21 | 12.35 |
| FAT1 | 4.81 | 11.82 | 25 | 3.91 | 3.12 | 11.11 |
| KMT2A | 6.73 | 4.55 | 25 | 7.03 | 1.04 | 11.11 |
| TSC1 | 2.88 | 12.73 | 12.5 | 1.56 | 4.17 | 5.56 |
| LRP1B | 6.73 | 8.18 | 31.25 | 6.25 | 7.29 | 8.02 |
| ELF3 | 9.62 | 7.27 | 18.75 | 14.06 | 5.21 | 4.94 |
| TRRAP | 4.81 | 6.36 | 25 | 6.25 | 5.21 | 5.56 |
| KRAS | 2.88 | 0.91 | 0 | 5.47 | 0 | 2.47 |
| RHOB | 2.88 | 3.64 | 0 | 5.47 | 0 | 6.79 |
| CREBBP | 10.58 | 6.36 | 18.75 | 9.38 | 4.17 | 6.79 |
| ERBB3 | 10.58 | 8.18 | 12.5 | 3.12 | 6.25 | 6.79 |
| ERBB2 | 4.81 | 8.18 | 18.75 | 10.16 | 7.29 | 7.41 |
| ZFP36L1 | 4.81 | 2.73 | 12.5 | 7.03 | 5.21 | 3.7 |

Optionally, process 500 proceeds to act 514, where the subject’s likelihood of responding to a therapy is identified using the UC mutational subtype identified at act 512. In some embodiments, when a subject is identified as having a UC mutational subtype TP53, ARID1A or HM at act 512, the subject is identified as having an increased likelihood of responding to an immunotherapy (e.g., an anti-PD-L1 antibody, such as atezolizumab) relative to a subject having other UC mutational subtypes, at act 514. In some embodiments, when a subject is identified as having a UC mutational subtype FGFR3-altered at act 512, the subject is identified as having an increased likelihood of responding to an anti-FGRF3 therapy relative to a subject having other UC mutational subtypes, at act 514. In some embodiments, when a subject is identified as having a UC mutational subtype KDM6A at act 512, the subject is identified as having an increased likelihood of responding to chemotherapy or radiotherapy relative to therapy with an ICI, at act 514.

In some embodiments, process 500 completes after act 512 completes. In some such embodiments, the determined UC mutational subtype and/or the identified likelihood the subject will respond to a therapy may be stored for subsequent use, provided to one or more recipients (e.g., a clinician, a researcher, etc.), and/or used to update the UC mutational subtype signature clusters.

However, in some embodiments, one or more other acts are performed after act 512. For example, in the illustrated embodiment of FIG. 5, process 500 may include one or more of optional acts 514 or 516 shown using dashed lines in FIG. 5. For example, at act 516, a prognosis may be identified for the subject.

It should be appreciated that although acts 514 and 516 are indicated as optional in the example of FIG. 5, in other embodiments, one or more other acts may be optional (in addition to or instead of acts 514 and 516). For example, in some embodiments, acts 502 and 504 may be optional (e.g., when the sequencing data is obtained and processed to obtain RNA expression data previously, process 500 may begin at act 506 by accessing the previously obtained RNA expression data). In some embodiments, the process 500 may comprise acts 502, 504, 506, 512 and 516, without act 514. In some embodiments, the process 500 may comprise acts 502, 504, 506, and 514 without act 516.

### Therapeutic Indications

Aspects of the disclosure relate to methods of identifying or selecting a therapeutic agent for a subject based upon determination of the subject’s urothelial cancer TME type or the subject UC mutational subtype. The disclosure is based, in part, on the recognition that subjects having certain UC TME types and/or UC mutational subtypes have an increased likelihood of responding to certain therapies (e.g., immunotherapeutic agents, anti-FGFR3 agents, platinum-based agents, etc.) relative to subjects having other UC TME types and/or UC mutational subtypes.

In some embodiments, the therapeutic agents are immuno-oncology (IO) agents. An IO agent may be a small molecule, peptide, protein (e.g., antibody, such as monoclonal antibody), interfering nucleic acid, or a combination of any of the foregoing. In some embodiments, the IO agents comprise a PD1 inhibitor, PD-L1 inhibitor, or PD-L2 inhibitor. Examples of IO agents include but are not limited to cemiplimab, nivolumab, pembrolizumab, avelumab, durvalumab, atezolizumab, BMS1166, BMS202, etc. In some embodiments, the IO agents comprise a combination of atezolizumab and albumin-bound paclitaxel, pembrolizumab and albumin-bound paclitaxel, pembrolizumab and paclitaxel, or pembrolizumab and Gemcitabine and Carboplatin.

In some embodiments, the therapeutic agents are anti-FGFR agents. An anti-FGFR agent may be a small molecule, peptide, protein (e.g., antibody, such as monoclonal antibody), interfering nucleic acid, or a combination of any of the foregoing. In some embodiments, an anti-FGFR agent is an anti-FGFR2 agent, or an anti-FGFR3 agent. In some embodiments, an anti-FGFR agent comprises lenvatinib, ponatinib, regorafenib, dovitinib, lucitanib, cediranib, intedanib, brivanib, futibatinib, or erdafitinib. In some embodiments, the anti-FGFR agent comprises erdafitinib. In some embodiments, the anti-FGFR agent comprises futibatinib.

In some embodiments, the therapeutic agents are platinum-based therapeutic agents. Examples of platinum-based therapeutic agents include but are not limited to cisplatin, carboplatin, and oxaliplatin. In some embodiments, the platinum-based therapeutic agent comprises cisplatin.

In some embodiments, the therapeutic agents are TGF-beta inhibitors. Examples of TGFbeta inhibitors include but are not limited to fresolimumab, LY2382770, galunisertib, and TEW-7197.

In some embodiments, the therapeutic agents are poly ADP ribose polymerase (PARP) inhibitors. Examples of PARP inhibitors include but are not limited to veliparib, fluzoparib, talazoparib, olaparib, rucaparib, and niraparib.

In some embodiments, methods described by the disclosure further comprise a step of administering one or more therapeutic agents to the subject based upon the determination of the subject’s TME type. In some embodiments, a subject is administered one or more (e.g., 1, 2, 3, 4, 5, or more) IO agents. In some embodiments, a subject is administered one or more (e.g., 1, 2, 3, 4, 5, or more) anti-FGFR agents. In some embodiments, a subject is administered one or more (e.g., 1, 2, 3, 4, 5, or more) platinum-based agents. In some embodiments, a subject is administered one or more (e.g., 1, 2, 3, 4, 5, or more) PARP inhibitors. In some embodiments, a subject is administered one or more (e.g., 1, 2, 3, 4, 5, or more) TGF-beta inhibitors.

Aspects of the disclosure relate to methods of treating a subject having (or suspected or at risk of having) urothelial cancer based upon a determination of the urothelial cancer TME type of the subject. In some embodiments, the methods comprise administering one or more (e.g., 1, 2, 3, 4, 5, or more) therapeutic agents to the subject. In some embodiments, the therapeutic agent (or agents) administered to the subject are selected from small molecules, peptides, nucleic acids, radioisotopes, cells (e.g., CAR T-cells, etc.), and combinations thereof. Examples of therapeutic agents include chemotherapies (e.g., cytotoxic agents, etc.), immunotherapies (e.g., immune checkpoint inhibitors, such as PD-1 inhibitors, PD-L1 inhibitors, etc.), antibodies (e.g., anti-HER2 antibodies), cellular therapies (e.g. CAR T-cell therapies), gene silencing therapies (e.g., interfering RNAs, CRISPR, etc.), antibody-drug conjugates (ADCs), and combinations thereof.

In some embodiments, a subject is administered an effective amount of a therapeutic agent. “An effective amount” as used herein refers to the amount of each active agent required to confer therapeutic effect on the subject, either alone or in combination with one or more other active agents. Effective amounts vary, as recognized by those skilled in the art, depending on the particular condition being treated, the severity of the condition, the individual patient parameters including age, physical condition, size, gender and weight, the duration of the treatment, the nature of concurrent therapy (if any), the specific route of administration and like factors within the knowledge and expertise of the health practitioner. These factors are well known to those of ordinary skill in the art and can be addressed with no more than routine experimentation. It is generally preferred that a maximum dose of the individual components or combinations thereof be used, that is, the highest safe dose according to sound medical judgment. It will be understood by those of ordinary skill in the art, however, that a patient may insist upon a lower dose or tolerable dose for medical reasons, psychological reasons, or for virtually any other reasons.

Empirical considerations, such as the half-life of a therapeutic compound, generally contribute to the determination of the dosage. For example, antibodies that are compatible with the human immune system, such as humanized antibodies or fully human antibodies, may be used to prolong half-life of the antibody and to prevent the antibody being attacked by the host’s immune system. Frequency of administration may be determined and adjusted over the course of therapy, and is generally (but not necessarily) based on treatment, and/or suppression, and/or amelioration, and/or delay of a cancer. Alternatively, sustained continuous release formulations of an anti-cancer therapeutic agent may be appropriate. Various formulations and devices for achieving sustained release are known in the art.

In some embodiments, dosages for an anti-cancer therapeutic agent as described herein may be determined empirically in individuals who have been administered one or more doses of the anti-cancer therapeutic agent. Individuals may be administered incremental dosages of the anti-cancer therapeutic agent. To assess efficacy of an administered anti-cancer therapeutic agent, one or more aspects of a cancer (e.g., tumor microenvironment, tumor formation, tumor growth, or TME types, etc.) may be analyzed.

Generally, for administration of any of the anti-cancer antibodies described herein, an initial candidate dosage may be about 2 mg/kg. For the purpose of the present disclosure, a typical daily dosage might range from about any of 0.1 µg/kg to 3 µg /kg to 30 µg /kg to 300 µg /kg to 3 mg/kg, to 30 mg/kg to 100 mg/kg or more, depending on the factors mentioned above. For repeated administrations over several days or longer, depending on the condition, the treatment is sustained until a desired suppression or amelioration of symptoms occurs or until sufficient therapeutic levels are achieved to alleviate a cancer, or one or more symptoms thereof. An exemplary dosing regimen comprises administering an initial dose of about 2 mg/kg, followed by a weekly maintenance dose of about 1 mg/kg of the antibody, or followed by a maintenance dose of about 1 mg/kg every other week. However, other dosage regimens may be useful, depending on the pattern of pharmacokinetic decay that the practitioner (e.g., a medical doctor) wishes to achieve. For example, dosing from one-four times a week is contemplated. In some embodiments, dosing ranging from about 3 µg /mg to about 2 mg/kg (such as about 3 µg /mg, about 10 µg /mg, about 30 µg /mg, about 100 µg /mg, about 300 µg /mg, about 1 mg/kg, and about 2 mg/kg) may be used. In some embodiments, dosing frequency is once every week, every 2 weeks, every 4 weeks, every 5 weeks, every 6 weeks, every 7 weeks, every 8 weeks, every 9 weeks, or every 10 weeks; or once every month, every 2 months, or every 3 months, or longer. The progress of this therapy may be monitored by conventional techniques and assays and/or by monitoring TME types as described herein. The dosing regimen (including the therapeutic used) may vary over time.

Dosing of immuno-oncology agents is well-known, for example as described by Louedec et al. Vaccines (Basel). 2020 Dec; 8(4): 632. For example, dosages of pembrolizumab, for example, include administration of 200 mg every 3 weeks or 400 mg every 6 weeks, by infusion over 30 minutes.

When the anti-cancer therapeutic agent is not an antibody, it may be administered at the rate of about 0.1 to 300 mg/kg of the weight of the patient divided into one to three doses, or as disclosed herein. In some embodiments, for an adult patient of normal weight, doses ranging from about 0.3 to 5.00 mg/kg may be administered. The particular dosage regimen, e.g., dose, timing, and/or repetition, will depend on the particular subject and that individual’s medical history, as well as the properties of the individual agents (such as the half-life of the agent, and other considerations well known in the art).

For the purpose of the present disclosure, the appropriate dosage of an anti-cancer therapeutic agent will depend on the specific anti-cancer therapeutic agent(s) (or compositions thereof) employed, the type and severity of cancer, whether the anti-cancer therapeutic agent is administered for preventive or therapeutic purposes, previous therapy, the patient’s clinical history and response to the anti-cancer therapeutic agent, and the discretion of the attending physician. Typically, the clinician will administer an anti-cancer therapeutic agent, such as an antibody, until a dosage is reached that achieves the desired result.

Administration of an anti-cancer therapeutic agent can be continuous or intermittent, depending, for example, upon the recipient’s physiological condition, whether the purpose of the administration is therapeutic or prophylactic, and other factors known to skilled practitioners. The administration of an anti-cancer therapeutic agent (e.g., an anti-cancer antibody) may be essentially continuous over a preselected period of time or may be in a series of spaced dose, e.g., either before, during, or after developing cancer.

As used herein, the term “treating” refers to the application or administration of a composition including one or more active agents to a subject, who has a cancer, a symptom of a cancer, or a predisposition toward a cancer, with the purpose to cure, heal, alleviate, relieve, alter, remedy, ameliorate, improve, or affect the cancer or one or more symptoms of urothelial cancer, or the predisposition toward urothelial cancer.

Alleviating urothelial cancer includes delaying the development or progression of the disease, or reducing disease severity. Alleviating the disease does not necessarily require curative results. As used therein, “delaying” the development of a disease (e.g., a cancer) means to defer, hinder, slow, retard, stabilize, and/or postpone progression of the disease. This delay can be of varying lengths of time, depending on the history of the disease and/or individuals being treated. A method that “delays” or alleviates the development of a disease, or delays the onset of the disease, is a method that reduces probability of developing one or more symptoms of the disease in a given time frame and/or reduces extent of the symptoms in a given time frame, when compared to not using the method. Such comparisons are typically based on clinical studies, using a number of subjects sufficient to give a statistically significant result.

“Development” or “progression” of a disease means initial manifestations and/or ensuing progression of the disease. Development of the disease can be detected and assessed using clinical techniques known in the art. Alternatively, or in addition to the clinical techniques known in the art, development of the disease may be detectable and assessed based on other criteria. However, development also refers to progression that may be undetectable. For purpose of this disclosure, development or progression refers to the biological course of the symptoms. “Development” includes occurrence, recurrence, and onset. As used herein “onset” or “occurrence” of a cancer includes initial onset and/or recurrence.

Examples of the antibody anti-cancer agents include, but are not limited to, alemtuzumab (Campath), trastuzumab (Herceptin), Ibritumomab tiuxetan (Zevalin), Brentuximab vedotin (Adcetris), Ado-trastuzumab emtansine (Kadcyla), blinatumomab (Blincyto), Bevacizumab (Avastin), Cetuximab (Erbitux), ipilimumab (Yervoy), nivolumab (Opdivo), pembrolizumab (Keytruda), atezolizumab (Tecentriq), avelumab (Bavencio), durvalumab (Imfinzi), and panitumumab (Vectibix).

Examples of an immunotherapy include, but are not limited to, a PD-1 inhibitor or a PD-L1 inhibitor, a CTLA-4 inhibitor, adoptive cell transfer, therapeutic cancer vaccines, oncolytic virus therapy, T-cell therapy, and immune checkpoint inhibitors.

Examples of radiation therapy include, but are not limited to, ionizing radiation, gamma-radiation, neutron beam radiotherapy, electron beam radiotherapy, proton therapy, brachytherapy, systemic radioactive isotopes, and radiosensitizers.

Examples of a surgical therapy include, but are not limited to, a curative surgery (e.g., tumor removal surgery), a preventive surgery, a laparoscopic surgery, and a laser surgery.

Examples of the chemotherapeutic agents include, but are not limited to, R-CHOP, Carboplatin or Cisplatin, Docetaxel, Gemcitabine, Nab-Paclitaxel, Paclitaxel, Pemetrexed, and Vinorelbine. Additional examples of chemotherapy include, but are not limited to, Platinating agents, such as Carboplatin, Oxaliplatin, Cisplatin, Nedaplatin, Satraplatin, Lobaplatin, Triplatin, Tetranitrate, Picoplatin, Prolindac, Aroplatin and other derivatives; Topoisomerase I inhibitors, such as Camptothecin, Topotecan, irinotecan/SN38, rubitecan, Belotecan, and other derivatives; Topoisomerase II inhibitors, such as Etoposide (VP-16), Daunorubicin, a doxorubicin agent (e.g., doxorubicin, doxorubicin hydrochloride, doxorubicin analogs, or doxorubicin and salts or analogs thereof in liposomes), Mitoxantrone, Aclarubicin, Epirubicin, Idarubicin, Amrubicin, Amsacrine, Pirarubicin, Valrubicin, Zorubicin, Teniposide and other derivatives; Antimetabolites, such as Folic family (Methotrexate, Pemetrexed, Raltitrexed, Aminopterin, and relatives or derivatives thereof); Purine antagonists (Thioguanine, Fludarabine, Cladribine, 6-Mercaptopurine, Pentostatin, clofarabine, and relatives or derivatives thereof) and Pyrimidine antagonists (Cytarabine, Floxuridine, Azacitidine, Tegafur, Carmofur, Capacitabine, Gemcitabine, hydroxyurea, 5-Fluorouracil (5FU), and relatives or derivatives thereof); Alkylating agents, such as Nitrogen mustards (e.g., Cyclophosphamide, Melphalan, Chlorambucil, mechlorethamine, Ifosfamide, mechlorethamine, Trofosfamide, Prednimustine, Bendamustine, Uramustine, Estramustine, and relatives or derivatives thereof); nitrosoureas (e.g., Carmustine, Lomustine, Semustine, Fotemustine, Nimustine, Ranimustine, Streptozocin, and relatives or derivatives thereof); Triazenes (e.g., Dacarbazine, Altretamine, Temozolomide, and relatives or derivatives thereof); Alkyl sulphonates (e.g., Busulfan, Mannosulfan, Treosulfan, and relatives or derivatives thereof); Procarbazine; Mitobronitol, and Aziridines (e.g., Carboquone, Triaziquone, ThioTEPA, triethylenemalamine, and relatives or derivatives thereof); Antibiotics, such as Hydroxyurea, Anthracyclines (e.g., doxorubicin agent, daunorubicin, epirubicin and relatives or derivatives thereof); Anthracenediones (e.g., Mitoxantrone and relatives or derivatives thereof); Streptomyces family antibiotics (e.g., Bleomycin, Mitomycin C, Actinomycin, and Plicamycin); and ultraviolet light.

In some aspects, the disclosure provides a method for treating urothelialcancer (UC), the method comprising administering one or more therapeutic agents (e.g., one or more anti-cancer agents, such as one or more immunotherapeutic agents) to a subject identified as having a particular urothelial cancer TME type, wherein the urothelial cancer TME type of the subject has been identified by method as described by the disclosure.

### Reports

In some aspects, methods disclosed herein comprise generating a report for assisting with the preparation of recommendation for prognosis and/or treatment. The generated report can provide summary of information, so that the clinician can identify the UC TME type or suitable therapy. The report as described herein may be a paper report, an electronic record, or a report in any format that is deemed suitable in the art. The report may be shown and/or stored on a computing device known in the art (e.g., handheld device, desktop computer, smart device, website, etc.). The report may be shown and/or stored on any device that is suitable as understood by a skilled person in the art.

In some embodiments, methods disclosed herein can be used for commercial diagnostic purposes. For example, the generated report may include, but is limited to, information concerning expression levels of one or more genes from any of the gene groups described herein, clinical and pathologic factors, patient’s prognostic analysis, predicted response to the treatment, classification of the UC TME environment (e.g., as belonging to one of the types described herein), the alternative treatment recommendation, and/or other information. In some embodiments, the methods and reports may include database management for the keeping of the generated reports. For instance, the methods as disclosed herein can create a record in a database for the subject (e.g., subject 1, subject 2, etc.) and populate the specific record with data for the subject. In some embodiments, the generated report can be provided to the subject and/or to the clinicians. In some embodiments, a network connection can be established to a server computer that includes the data and report for receiving or outputting. In some embodiments, the receiving and outputting of the date or report can be requested from the server computer.

### Computer Implementation

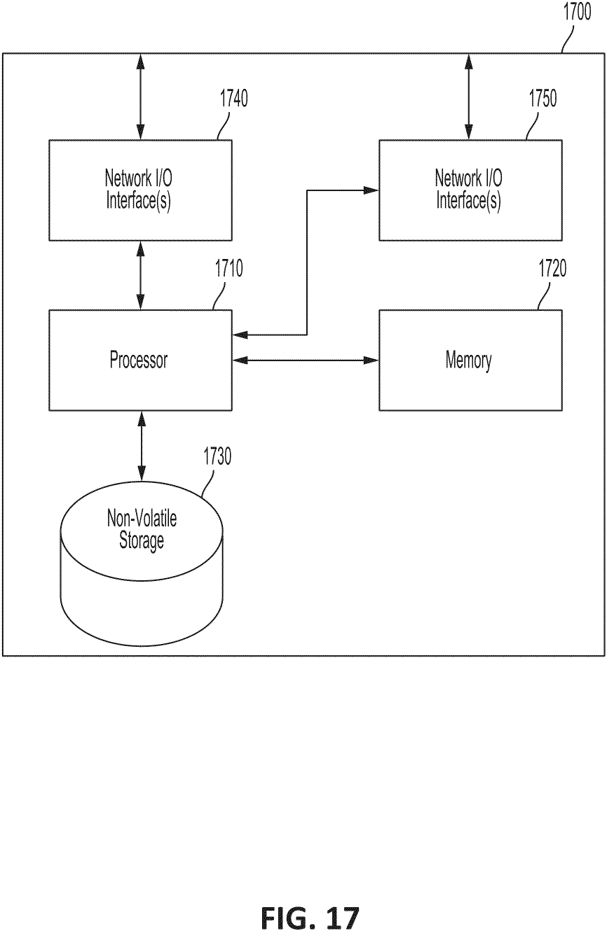

An illustrative implementation of a computer system 1700 that may be used in connection with any of the embodiments of the technology described herein (e.g., such as the method of FIG. 1, FIG. 2, FIG. 3, FIG. 5, etc.) is shown in FIG. 17. The computer system 1700 includes one or more processors 1710 and one or more articles of manufacture that comprise non-transitory computer-readable storage media (e.g., memory 1720 and one or more non-volatile storage media 1730). The processor 1710 may control writing data to and reading data from the memory 1720 and the non-volatile storage device 1730 in any suitable manner, as the aspects of the technology described herein are not limited to any particular techniques for writing or reading data. To perform any of the functionality described herein, the processor 1710 may execute one or more processor-executable instructions stored in one or more non-transitory computer-readable storage media (e.g., the memory 1720), which may serve as non-transitory computer-readable storage media storing processor-executable instructions for execution by the processor 1710.

Computing device 1700 may also include a network input/output (I/O) interface 1740 via which the computing device may communicate with other computing devices (e.g., over a network), and may also include one or more user I/O interfaces 1750, via which the computing device may provide output to and receive input from a user. The user I/O interfaces may include devices such as a keyboard, a mouse, a microphone, a display device (e.g., a monitor or touch screen), speakers, a camera, and/or various other types of I/O devices.

The above-described embodiments can be implemented in any of numerous ways. For example, the embodiments may be implemented using hardware, software, or a combination thereof. When implemented in software, the software code can be executed on any suitable processor (e.g., a microprocessor) or collection of processors, whether provided in a single computing device or distributed among multiple computing devices. It should be appreciated that any component or collection of components that perform the functions described above can be generically considered as one or more controllers that control the above-discussed functions. The one or more controllers can be implemented in numerous ways, such as with dedicated hardware, or with general purpose hardware (e.g., one or more processors) that is programmed using microcode or software to perform the functions recited above.

In this respect, it should be appreciated that one implementation of the embodiments described herein comprises at least one computer-readable storage medium (e.g., RAM, ROM, EEPROM, flash memory or other memory technology, CD-ROM, digital versatile disks (DVD) or other optical disk storage, magnetic cassettes, magnetic tape, magnetic disk storage or other magnetic storage devices, or other tangible, non-transitory computer-readable storage medium) encoded with a computer program (i.e., a plurality of executable instructions) that, when executed on one or more processors, performs the above-discussed functions of one or more embodiments. The computer-readable medium may be transportable such that the program stored thereon can be loaded onto any computing device to implement aspects of the techniques discussed herein. In addition, it should be appreciated that the reference to a computer program which, when executed, performs any of the above-discussed functions, is not limited to an application program running on a host computer. Rather, the terms computer program and software are used herein in a generic sense to reference any type of computer code (e.g., application software, firmware, microcode, or any other form of computer instruction) that can be employed to program one or more processors to implement aspects of the techniques discussed herein.

The foregoing description of implementations provides illustration and description but is not intended to be exhaustive or to limit the implementations to the precise form disclosed. Modifications and variations are possible in light of the above teachings or may be acquired from practice of the implementations. In other implementations the methods depicted in these figures may include fewer operations, different operations, differently ordered operations, and/or additional operations. Further, non-dependent blocks may be performed in parallel.

It will be apparent that example aspects, as described above, may be implemented in many different forms of software, firmware, and hardware in the implementations illustrated in the figures. Further, certain portions of the implementations may be implemented as a “module” that performs one or more functions. This module may include hardware, such as a processor, an application-specific integrated circuit (ASIC), or a field-programmable gate array (FPGA), or a combination of hardware and software.

Having thus described several aspects and embodiments of the technology set forth in the disclosure, it is to be appreciated that various alterations, modifications, and improvements will readily occur to those skilled in the art. Such alterations, modifications, and improvements are intended to be within the spirit and scope of the technology described herein. For example, those of ordinary skill in the art will readily envision a variety of other means and/or structures for performing the function and/or obtaining the results and/or one or more of the advantages described herein, and each of such variations and/or modifications is deemed to be within the scope of the embodiments described herein. Those skilled in the art will recognize or be able to ascertain using no more than routine experimentation many equivalents to the specific embodiments described herein. It is, therefore, to be understood that the foregoing embodiments are presented by way of example only and that, within the scope of the appended claims and equivalents thereto, inventive embodiments may be practiced otherwise than as specifically described. In addition, any combination of two or more features, systems, articles, materials, kits, and/or methods described herein, if such features, systems, articles, materials, kits, and/or methods are not mutually inconsistent, is included within the scope of the present disclosure.

The above-described embodiments can be implemented in any of numerous ways. One or more aspects and embodiments of the present disclosure involving the performance of processes or methods may utilize program instructions executable by a device (e.g., a computer, a processor, or other device) to perform, or control performance of, the processes or methods. In this respect, various inventive concepts may be embodied as a computer readable storage medium (or multiple computer readable storage media) (e.g., a computer memory, one or more floppy discs, compact discs, optical discs, magnetic tapes, flash memories, circuit configurations in Field Programmable Gate Arrays or other semiconductor devices, or other tangible computer storage medium) encoded with one or more programs that, when executed on one or more computers or other processors, perform methods that implement one or more of the various embodiments described above. The computer readable medium or media can be transportable, such that the program or programs stored thereon can be loaded onto one or more different computers or other processors to implement various ones of the aspects described above. In some embodiments, computer readable media may be non-transitory media.

The terms “program” or “software” are used herein in a generic sense to refer to any type of computer code or set of computer-executable instructions that can be employed to program a computer or other processor to implement various aspects as described above. Additionally, it should be appreciated that according to one aspect, one or more computer programs that when executed perform methods of the present disclosure need not reside on a single computer or processor, but may be distributed in a modular fashion among a number of different computers or processors to implement various aspects of the present disclosure.

Computer-executable instructions may be in many forms, such as program modules, executed by one or more computers or other devices. Generally, program modules include routines, programs, objects, components, data structures, etc. that perform particular tasks or implement particular abstract data types. Typically the functionality of the program modules may be combined or distributed as desired in various embodiments.

Also, data structures may be stored in computer-readable media in any suitable form. For simplicity of illustration, data structures may be shown to have fields that are related through location in the data structure. Such relationships may likewise be achieved by assigning storage for the fields with locations in a computer-readable medium that convey relationship between the fields. However, any suitable mechanism may be used to establish a relationship between information in fields of a data structure, including through the use of pointers, tags or other mechanisms that establish relationship between data elements.

When implemented in software, the software code can be executed on any suitable processor or collection of processors, whether provided in a single computer or distributed among multiple computers.

Further, it should be appreciated that a computer may be embodied in any of a number of forms, such as a rack-mounted computer, a desktop computer, a laptop computer, or a tablet computer, as non-limiting examples. Additionally, a computer may be embedded in a device not generally regarded as a computer but with suitable processing capabilities, including a Personal Digital Assistant (PDA), a smartphone, a tablet, or any other suitable portable or fixed electronic device.

Also, a computer may have one or more input and output devices. These devices can be used, among other things, to present a user interface. Examples of output devices that can be used to provide a user interface include printers or display screens for visual presentation of output and speakers or other sound generating devices for audible presentation of output. Examples of input devices that can be used for a user interface include keyboards, and pointing devices, such as mice, touch pads, and digitizing tablets. As another example, a computer may receive input information through speech recognition or in other audible formats.

Such computers may be interconnected by one or more networks in any suitable form, including a local area network or a wide area network, such as an enterprise network, and intelligent network (IN) or the Internet. Such networks may be based on any suitable technology and may operate according to any suitable protocol and may include wireless networks, wired networks or fiber optic networks.

Also, as described, some aspects may be embodied as one or more methods. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

TABLE 6

Exemplary NCBI Accession Numbers for genes listed in Tables

| | |
|---|---|
| HLA-C | NM_002117; NM_001243042 |
| TAPBP | XM_017011227; XM_047419272; NM_001410875; XM_047419271; XM_011514828; NM_003190; NM_172208; NM_172209 |
| HLA-B | NM_005514 |
| B2M | NM_004048; XM_005254549 |
| TAP2 | NM_001290043; NM_000544; NM_018833 |
| HLA-A | XM_041680767; NM_001242758; XM_041680768; NM_002116 |
| TAP1 | NM_000593; NM_001292022 |
| NLRC5 | NM_001330552; NM_001384961; NM_001384969; NM_001384972; NM_001384973; NR_169518; NM_001384951; NM_001384959; NM_032206; NR_169513; XR_001752000; XM_006721300; XM_017023770; XM_047434760; XM_047434765; NM_001384950; NM_001384958; NM_001384964; NR_169520; NM_001384954; NM_001384966; NR_169514; NR_169517; XM_047434761; XM_047434763; NM_001384967; NM_001384971; XM_047434766; NM_001384965; NM_001384970; NR_169512; NR_169519; XM_047434762; XM_047434764; NM_001384953; NM_001384956; NM_001384957; NM_001384962; NM_001384963; NR_169515; NR_169516; NM_001384952; NM_001384955; NM_001384960; NM_001384968 |
| HLA-DQB1 | NM_001243962; NM_001243961; NM_002123 |
| HLA-DMA | NM_006120 |
| HLA-DMB | NM_002118 |
| HLA-DRA | NM_019111 |
| CIITA | XM_047434115; NM_001379332; XR_007064880; XM_006720880; XM_011522491; XM_047434119; NM_001379334; XM_047434118; XM_047434120; XM_047434123; NM_001379333; XM_011522486; NM_000246; NM_001286402; XM_047434122; XM_047434126; XR_001751904; XR_007064879; XM_047434114; XM_047434117; XM_047434125; NM_001286403; NM_001379331; XM_011522485; XM_047434127; XM_047434128; NR_104444; XM_011522484; XM_011522490; XM_047434116; XM_047434124; NM_001379330 |
| HLA-DQA1 | NM_002122; XM_006715079 |
| HLA-DPB1 | NM_002121 |
| HLA-DRB 1 | XM_024452553; NM_001359194; XM_047444767; XM_047444769; NM_001243965; NM_002124; XM_047444770; NM_001359193; XM_047443024; XM_047444768 |
| HLA-DPA1 | NM_001405020; NM_001242525; NM_033554; NM_001242524; XM_047418717 |
| TNFRSF4 | XM_011542074; NM_003327; XR_007063145; NM_001410709; XM_011542077; XM_011542075; XM_011542076 |
| CD27 | NM_001413263; NM_001413264; NM_001413265; NM_001413267; NM_001242; XM_011521042; XM_017020234; NM_001413266; NM_001413268; NR_182127; NR_182125; XM_047429900; NR_182126; NR_182128 |
| CD80 | NM_005191 |
| CD40LG | NM_000074 |
| TNFRSF9 | NM_001561; XM_047419672; XM_006710618 |
| CD40 | NM_001302753; NM_001322422; NM_152854; NM_001322421; NM_001362758; NM_001250; XM_047440601; NR_136327; XM_011529109; XM_005260619; XM_017028135; XM_017028136; NR_126502 |
| CD28 | NM_006139; NM_001243078; NM_001410981; NM_001243077; XM_011512194 |
| ICOSLG | NM_001395918; XM_011529514; NM_001283050; NM_001283051; NM_001283052; XM_047440732; NM_015259; XM_047440731; NM_001365759; XM_047440730; XM_011529516; XM_047440729 |
| CD83 | NM_001040280; NM_001251901; NM_004233 |
| TNFSF9 | NM_003811 |
| CD70 | NM_001252; NM_001330332 |
| TNFSF4 | XM_047429908; NM_003326; XM_047429896; NM_001297562; XM_047429902; XM_017002228; XM_011509964 |
| ICOS | XR_007073112; XM_047444022; NM_012092 |
| CD86 | NM_001206924; NM_006889; NM_176892; NM_001206925; NM_175862 |
| ZAP70 | NM_001378594; NM_207519; XR_007081582; NM_001079; XM_047445775; XM_047445774; XM_047445776; XR_007081583 |
| GZMB | NM_001346011; NM_004131; NR_144343 |
| GZMK | NM_002104 |
| IFNG | NM_000619 |
| FASLG | NM_001302746; NM_000639 |
| EOMES | NM_001278182; XM_005265510; NM_005442; NM_001278183 |
| TBX21 | NM_013351 |
| GZMA | NM_006144 |
| CD8A | NM_001382698; NM_001145873; NM_001768; NR_168478; NR_168479; NM_171827; NR_168480; NR_168481; NR_027353 |
| GNLY | XM_005264085; NM_001302758; XM_047442947; NM_006433; XM_005264084; NM_012483 |
| PRF1 | NM_005041; NM_001083116 |
| CD8B | NM_172102; NM_172100; NM_001178100; NM_004931; NM_172101; NM_172213; NM_172099; XM_011533164 |
| NKG7 | XM_006723228; XM_005258955; NM_001363693; NM_005601 |
| FGFBP2 | NM_031950 |
| CD244 | NM_001166663; XM_047422535; XM_011509622; NM_016382; NM_001166664; XM_011509623; XM_011509621 |
| KLRK1 | NM_007360.4 |
| KIR2DL4 | NM_001080770; NM_001080772; NM_002255; NM_001258383 |
| CD226 | NM_006566; XM_047437274; NM_001303619; XM_047437275; XM_047437276; XM_006722374; XM_005266642; XM_047437277; NM_001303618 |
| KLRF1 | NM_001291822; XM_017019415; XM_047428956; NM_001366534; NR_120305; NM_001291823; NM_016523; NR_159359; NR_159360; NR_159361 |
| KLRC2 | NM_002260 |
| NCR1 | NM_004829; NM_001145457; XM_011527530; XM_047439727; NM_001242357; XM_011527529; NM_001242356; NM_001145458 |
| GZMH | NM_001270781; NM_001270780; NM_033423 |
| SH2D1B | NM_053282 |
| NCR3 | NM_001145467; XM_011514459; XM_006715049; NM_001145466; NM_147130 |
| CD160 | NM_007053; XM_005272929; XM_011509104; NR_103845 |
| TRBC2 | NG_001333.2 |
| CD3E | NM_000733 |
| CD3G | XM_005271724; XM_006718941; NM_000073 |
| ITK | NM_005546 |
| TRBC1 | NG_001333.2 |
| TRAT1 | NM_016388; NM_001317747 |
| CD5 | NM_014207; NM_001346456 |
| TRAC | NG_001332.3 |
| CD3D | NM_001040651; NM_000732 |
| IL21 | NM_021803; NM_001207006 |
| IL12RB2 | NR_047584; XM_011541384; XM_047419669; XM_047419670; XM_005270827; XM_006710617; NM_001374259; XM_011541383; XM_047419667; NM_001258215; NM_001258216; XM_047419665; XM_047419666; XM_047419668; NM_001258214; NM_001319233; XM_005270828; XM_017001203; NM_001559; NR_047583 |
| IL2 | NM_000586 |
| STAT4 | XM_047445601; XM_047445609; XM_047445602; XM_047445604; NM_003151; XM_006712719; XM_047445606; XM_047445605; XM_047445607; NM_001243835; XM_047445603; XM_047445608; XM_047445600 |
| IL13 | NM_001354991; NM_001354992; NM_002188; NM_001354993 |
| CCR4 | XM_017005687; NM_005508 |
| IL10 | NM_000572; NR_168467; NR_168466; NM_001382624 |
| IL4 | NM_000589; NM_001354990; NM_172348 |
| IL5 | XM_047417148; XM_005271988; XM_011543373; NM_000879 |
| CD22 | NM_001185100; NM_001185099; NM_024916; NM_001185101; NM_001771; NM_001278417 |
| TNFRSF13C | NM_052945 |
| STAP1 | NM_012108; NM_001317769 |
| CD79B | NM_001039933; NM_021602; NM_000626; NM_001329050 |
| PAX5 | NM_001280547; NM_001280553; NM_016734; NM_001280548; NR_103999; NM_001280551; NM_001280555; NM_001280554; NM_001280552; NM_001280556; NM_001280549; NM_001280550; NR_104000 |
| CR2 | NM_001877; NM_001006658 |
| TNFRSF13B | NM_012452 |
| CD79A | NM_021601; NM_001783 |
| TNFRSF17 | NM_001192 |
| FCRL5 | XM_011510032; XM_047431422; XM_011510030; XM_011510033; XM_011510031; NM_031281; NM_001195388 |
| MS4A1 | NM_021950; NM_152866; NM_152867 |
| CD19 | NM_001178098; NM_001385732; NM_001770; XR_950871; NR_169755; XM_011545981 |
| BLK | XM_047422081; NM_001330465; XM_011543829; XM_011543824; XM_011543827; XM_047422083; XM_047422084; XM_011543828; XM_047422082; NM_001715; XM_011543825 |
| MRC1 | NM_002438; NM_001009567 |
| SIGLEC1 | NM_001367089; NM_023068 |
| MSR1 | NM_138716; NM_002445; XM_024447161; NM_138715; NM_001363744 |
| CD163 | XM_047429895; XM_024449278; NM_203416; NM_001370145; NM_001370146; NM_004244; NR_163255 |
| CSF1R | NM_001375320; NM_005211; NR_164679; NM_001349736; NM_001288705; NM_001375321; NR_109969 |
| CD68 | NM_001251; NM_001040059 |
| IL4I1 | NM_001385639; NM_172374; NM_152899; NR_047577; NM_001258018; NM_001258017 |
| CMKLR1 | NM_001142343; XM_047428313; NM_001142345; NM_001142344; NM_004072 |
| SOCS3 | NM_001378933; NM_003955; NM_001378932 |
| IRF5 | XM_047420336; NM_001242452; XM_006715974; NM_001364314; NM_032643; XM_011516160; XM_011516158; XM_047420340; NM_001347928; XM_047420337; NM_001098629; XM_011516159; XM_047420338; NM_001098627; NM_001098630; XM_047420339 |
| NOS2 | NM_153292; NM_000625 |
| IL1B | NM_000576; XM_047444175 |
| IL12B | NM_002187 |
| IL23A | NM_016584 |
| TNF | NM_000594 |
| IL12A | NM_000882; NM_001354583; NM_001354582; NM_001397992 |
| CCL3 | NR_168496; NR_168495; NM_002983; NR_168494 |
| IFNB 1 | NM_002176 |
| IFNA2 | NM_000605 |
| TNFSF10 | NR_033994; NM_001190943; NM_003810; NM_001190942 |
| PDCD1 | NM_005018; XM_006712573 |
| BTLA | NM_001085357; NM_181780; XM_011512447; XM_017005748; XM_047447496 |
| HAVCR2 | NM_032782 |
| CD274 | XM_047423262; NM_001314029; NM_001267706; NR_052005; NM_014143 |
| VSIR | NM_022153 |
| LAG3 | XM_047428839; NM_002286; NM_001414177; XM_011520956; NM_001414176 |
| TIGIT | XM_047447672; XM_047447671; NM_173799 |
| PDCD1LG2 | XM_005251600; NM_025239 |
| CTLA4 | NM_005214; NM_001037631 |
| IKZF2 | XM_011510804; XM_011510815; NM_001371277; XM_011510803; XM_011510808; XM_011510816; NM_001371275; XM_005246386; XM_011510807; XM_011510810; XM_047443723; XM_011510811; NM_001371276; XM_011510809; XM_047443722; XM_047443724; NM_001079526; NM_001371274; NM_001387220; NM_016260; XM_011510817; XM_011510818; XM_011510805; XM_011510812; XM_011510819; XM_047443721; XM_047443725; XM_047443727; XM_011510802; XM_047443726 |
| TNFRSF18 | NM_148901; NM_004195; XM_017002722; NM_148902 |
| FOXP3 | NM_001114377; NM_014009 |
| CCR8 | NM_005201 |
| IKZF4 | XM_017019810; XM_047429342; XM_047429345; XM_047429347; NM_001351089; NM_022465; XM_047429341; XM_047429346; XM_047429349; XM_047429351; NM_001351090; NM_001351091; XM_011538664; XM_011538669; XM_047429350; XM_047429352; XM_047429353; XM_017019806; XM_047429348; XM_005269089; XM_047429344; NM_001351092; XM_017019812 |
| CD177 | XM_017027021; XM_017027022; NM_020406 |
| FFAR2 | XM_047438699; NM_005306; NM_001370087; XM_017026711; XM_047438700 |
| PGLYRP1 | NM_005091 |
| CXCR1 | NM_000634 |
| MPO | NM_000250 |
| CXCR2 | XM_047444190; XM_047444188; NM_001168298; NM_001557; XM_005246530; XM_047444189; XM_017003991; XM_047444191; XM_047444187 |
| ELANE | NM_001972 |
| CTSG | NM_001911; XM_011536499 |
| PRTN3 | XM_011528136; NM_002777 |
| FCGR3B | NM_001271036; NM_001271037; NM_001244753; NM_000570; NM_001271035 |
| ARG1 | NM_001369020; NM_000045; NM_001244438; NR_160934 |
| IL6 | NM_001318095; NM_000600; NM_001371096; XM_005249745 |
| CYBB | XM_047441855; NM_000397 |
| PTGS2 | NM_000963 |
| IDO1 | NM_002164 |
| TGFB2 | NM_003238; NR_138149; NR_138148; NM_001135599 |
| MIF | NM_002415 |
| TGFB3 | NM_001329938; NM_003239; NM_001329939 |
| TGFB1 | NM_000660; XM_011527242 |
| IL22 | NM_020525 |
| COL6A3 | NM_057164; NM_057167; NM_057166; NM_004369; NM_057165 |
| PDGFRB | NM_001355016; NM_002609; NM_001355017; NR_149150 |
| COL6A1 | NM_001848 |
| MFAP5 | NM_001297709; NR_123733; NR_123734; NM_001297711; NM_003480; NM_001297710; NM_001297712 |
| COL5A1 | XM_017014266; NM_000093; NM_001278074 |
| FAP | XM_011510797; NM_004460; XM_011510796; XM_017003585; XR_001738668; XR_922891; NM_001291807 |
| PDGFRA | XM_047415767; NM_001347828; NM_001347829; XM_005265743; XM_017008281; NM_001347827; XM_047415766; NM_001347830; NM_006206; XM_006714041 |
| FGF2 | NM_001361665; NM_002006 |
| ACTA2 | NM_001406467; NM_001141945; NM_001406463; NM_001406464; NM_001406471; NM_001406466; NM_001406462; NM_001320855; NM_001613; NM_001406468; NM_001406469 |
| COL6A2 | NM_001849; NM_058175; NM_058174 |
| FBLN1 | NM_006485; NM_006486; NM_001996; NM_006487 |
| CD248 | NM_020404 |
| COL1A1 | XM_005257058; XM_005257059; XM_011524341; NM_000088 |
| MMP2 | NM_001302509; NM_001127891; NM_001302508; NM_001302510; NM_004530 |
| COL1A2 | NM_000089 |
| MMP3 | NM_002422 |
| LUM | NM_002345 |
| CXCL12 | NM_000609; NM_001277990; NM_199168; NM_001178134; NM_001033886 |
| LRP1 | NM_002332 |
| LAMC2 | NM_005562; NM_018891; XM_047420361; XM_047420358; XM_017001273 |
| TNC | XM_005251975; XM_006717096; XM_011518628; XM_017014681; XM_047423311; XM_047423321; XM_047423323; XM_047423328; XM_011518626; XM_047423312; XM_047423313; XM_047423317; XM_047423318; XM_005251973; XM_006717098; XM_047423322; XM_047423324; XM_047423327; XM_005251972; XM_006717097; XM_011518629; XM_047423309; XM_047423314; XM_047423325; NM_001410991; XM_017014680; XM_047423315; XM_047423329; XM_011518625; XM_017014679; XM_047423310; XM_047423320; XM_047423330; XM_047423331; XM_005251974; XM_006717101; XM_047423316; XM_047423319; XM_047423326; XM_047423332; XM_017014678; XM_024447530; NM_002160 |
| COL11A1 | XM_017000337; XM_017000335; XM_017000336; NR_134980; NM_080629; XR_007085257; XM_017000334; NM_001190709; NM_001854; NM_080630 |
| VTN | NM_000638 |
| LAMB3 | XM_005273124; XM_047420351; NM_001127641; XM_017001272; NM_000228; NM_001017402 |
| FN1 | NM_001306129; NM_001365519; NM_212474; NM_001306132; NM_001365517; NM_001365522; NM_001306131; NM_001365521; NM_212476; NM_212478; NM_212475; NM_001365523; NM_001365524; NM_002026; NM_001365520; NM_212482; NM_001365518; NM_054034; NM_001306130 |
| LAMA3 | XM_011525981; XM_017025743; XM_047437504; NM_001127717; NM_000227; XM_011525978; XM_011525979; XM_047437503; NM_198129; XM_011525980; XM_017025744; XM_047437506; XM_011525982; XM_047437505; NM_001302996; NR_130106; NM_001127718 |
| LGALS9 | XM_011524796; NM_001330163; NR_024043; XM_006721893; XM_006721895; NM_002308; XM_006721892; NM_009587 |
| COL4A1 | NM_001845; NM_001303110 |
| ELN | XM_011515869; XM_011515873; XM_017011814; XM_047419961; XM_047419973; XM_047419978; XM_005250187; XM_011515871; XM_011515872; XM_047419958; XM_047419962; XM_047419963; XM_047419965; NM_001278914; XM_005250188; XM_011515874; XM_047419957; XM_047419964; XM_047419966; XM_047419974; XM_047419979; NM_000501; NM_001278912; NM_001278939; XM_011515877; XM_047419960; XM_047419967; XM_047419971; XM_047419977; NM_001081753; XM_011515876; XM_047419955; XM_047419970; XM_047419975; NM_001081754; NM_001278917; XM_017011813; XM_047419954; XM_047419956; XM_047419968; XM_047419969; XM_047419972; XM_047419980; NM_001278915; NM_001278918; XM_011515868; XM_011515870; XM_047419981; NM_001081755; NM_001278916; XM_011515875; XM_047419959; XM_047419976; NM_001081752; NM_001278913 |
| LGALS7 | NM_002307.4 |
| COL3A1 | NM_000090; NM_001376916 |
| ADAMTS4 | NM_001320336; NM_005099; XM_047434904 |
| ADAMTS5 | XM_047440680; NM_007038 |
| CA9 | XM_047423849; NM_001216; XM_047423850 |
| LOX | NM_001317073; NM_002317; NM_001178102 |
| MMP1 | NM_001145938; NM_002421 |
| MMP11 | NM_005940; NR_133013 |
| MMP12 | NM_002426 |
| MMP7 | NM_002423 |
| MMP9 | NM_004994 |
| PLOD2 | XM_017006625; NM_000935; XM_047448320; NM_182943; XM_047448319 |
| VEGFC | NM_005429 |
| VEGFA | NM_001171625; NM_003376; NM_001033756; NM_001171624; NM_001171626; NM_001171630; NM_001025366; NM_001317010; NM_001025368; NM_001025370; NM_001171623; NM_001171622; NM_001171628; NM_001171629; NM_001204385; NM_001025367; NM_001025369; NM_001171627; NM_001204384; NM_001287044 |
| PDGFC | XM_047415970; XM_017008455; NM_016205; XM_047415971; XM_047415969; XM_047415972; NR_036641 |
| KDR | NM_002253 |
| CDH5 | XM_047433469; XM_047433470; NM_001114117; NM_001795; XM_047433471; XM_011522801 |
| VEGFB | NM_003377; NM_001243733 |
| PGF | NM_001293643; NM_002632; NM_001207012; XM_047431476 |
| TEK | NM_001375475; NM_000459; NM_001290077; NM_001290078; NM_001375476 |
| ANGPT2 | NM_001118888; NM_001386335; NM_001386337; NM_001118887; NM_001147; NM_001386336 |
| FLT1 | NM_001160030; NM_001159920; XM_011535014; XM_017020485; NM_001160031; NM_002019 |
| CXCL8 | NM_000584; NM_001354840 |
| VWF | NM_000552; XM_047429501 |
| ANGPT1 | NM_001314051; NM_001146; NM_001199859; XM_047421699; NM_139290 |
| CXCL5 | NM_002994 |
| NOS3 | NM_001160110; NM_000603; NM_001160109; NM_001160111 |
| VCAM1 | NM_080682; NM_001078; NM_001199834 |
| MMRN1 | NM_001410735; NM_001371403; XM_047449832; XM_047449831; NM_007351 |
| CLEC14A | NM_175060 |
| ENG | NM_000118; NM_001406715; NM_001114753; NM_001278138 |
| MMRN2 | NM_024756 |
| CCND1 | NM_053056 |
| CCNB1 | NM_001354844; NM_031966; NM_001354845 |
| CETN3 | NM_004365; NM_001297765; NM_001297768 |
| CDK2 | NM_001290230; XM_011537732; NM_052827; NM_001798 |
| E2F1 | XM_047439961; NM_005225 |
| AURKA | XM_047440428; XM_047440427; NM_001323304; NM_001323303; NM_198435; NM_198437; NM_198433; NM_198434; NM_198436; XM_017028034; XM_017028035; NM_001323305; NM_003600 |
| BUB1 | NM_004336; NM_001278617; XM_047445616; NM_001278616 |
| AURKB | NM_001313950; NM_001313953; XM_017025311; XM_047437050; NM_001313952; NM_004217; NM_001313954; NR_132730; NR_132731; NM_001284526; XM_047437051; XM_011524072; NM_001256834; NM_001313951; NM_001313955 |
| PLK1 | NM_005030 |
| MCM6 | NM_005915 |
| ESCO2 | NM_001017420; XR_949378; XR_007060703; XM_011544422; XM_011544421 |
| MYBL2 | NM_002466; NM_001278610 |
| MKI67 | NM_002417; NM_001145966; XM_006717864; XM_011539818 |
| MCM2 | NM_004526; XM_024453531; NR_073375 |
| CCNE1 | NM_001238; XM_011527440; NM_001322259; NM_001322261; XM_047439606; NM_001322262; NM_057182 |
| CDH2 | XM_011525788; NM_001308176; XM_017025514; NM_001792 |
| ZEB1 | XM_047425691; NM_001174096; NM_001323650; NM_001323655; NM_001323660; NM_001323677; XM_047425683; XM_047425688; NM_001174095; NM_001323638; NM_001323645; NM_001323647; NM_001323663; NM_001323666; NM_001323672; NM_030751; XM_047425684; XM_047425686; NM_001323662; XM_047425685; XM_047425682; XM_047425689; XM_047425693; XM_047425695; NM_001128128; NM_001174094; NM_001323641; NM_001323644; NM_001323659; NM_001323674; NM_001323675; NM_001323676; XM_047425687; NM_001323642; NM_001323648; NM_001323649; NM_001323652; NM_001323654; NM_001323671; XM_047425690; NM_001323643; NM_001323658; NM_001323665; NM_001323651; NM_001323656; NM_001323661; NM_001323664; NM_001323673; NM_001323678; XM_047425692; XM_047425694; XM_047425696; NM_001174093; NM_001323646; NM_001323653; NM_001323657 |
| ZEB2 | NM_001171653; NM_014795; NR_033258 |
| TWIST1 | NR_149001; NM_000474 |
| SNAI1 | NM_005985 |
| SNAI2 | NM_003068 |
| TWIST2 | NM_057179; XR_007069137; NM_001271893 |
| PWRN1 | NR_026646.1; XR_007069208.1; XR_007069209.1; XR_007069210.1; XR_007069211.1; XR_007069212.1; XR_007069213.1; XR_007069214.1; XR_007069215.1; XR_007069216.1; XR_007069217.1 |
| PWRN3 | NR_130780.1 |
| GSTM5 | NM_000851; XM_005270785; XM_005270784 |
| GSTM4 | NM_000850; NM_147148; XR_007059238; NM_147149; XM_017001085; XM_047418652; XM_047418654; NR_024538 |
| GSTM2 | XR_007070522; NM_000848; NM_001142368; XR_007070521; XM_047443675; XR_007059237; XR_007059236 |
| ZNF321P | NM_001202473 |
| ZNF320 | XM_047438301; XM_047438300; XM_047438309; XM_047438310; NM_001351775; XM_047438305; NM_001387574; XM_024451397; NM_001387576; XM_024451396; XM_047438304; XM_047438306; XM_047438307; NM_001351774; NM_001351776; NM_001387575; NM_001387577; XM_047438303; XM_047438308; NM_001387572; NM_001351773; NM_001351777; XR_002958275; NM_001387571; NM_001387570; NM_207333; XM_047438302; NM_001387573 |
| ZNF66 | NM_001355197.2; XM_047439350.1 |
| ZNF737 | XM_006722597; XM_047438007; NM_001159293; XM_011527598; XM_017026117; XM_005259697; XM_047438006; XM_011527599; XM_047438008 |
| KRT20 | NM_019010 |
| UPK1B | NM_006952 |
| FOXA1 | NM_004496 |
| ACER2 | XM_017014694; NM_001010887; XM_047423336; XM_011517858; XM_047423335; XR_002956780; XR_002956781; XM_011517859 |
| SEMA5A | XM_011514155; XM_011514158; XM_047417865; XM_006714506; XM_011514159; XM_047417866; XM_006714507; XM_011514157; XM_047417867; NM_003966; XM_011514156 |
| PPARG | NM_001354669; NM_001354670; NM_001374263; NM_001330615; NM_001374262; NM_005037; NM_001374261; NM_138711; NM_138712; NM_001374264; NM_001374266; NM_001354668; NM_015869; NM_001354666; NM_001354667; NM_001374265 |
| GATA3 | XM_005252443; XM_047425044; NM_002051; XM_005252442; XM_047425045; NM_001002295 |
| SNX31 | XM_024447085; XM_011516899; XM_017013154; XM_017013161; XM_017013163; XM_024447086; XM_017013157; XM_017013165; XM_017013162; XM_017013158; NM_152628; XM_017013153; XM_017013156; XM_017013159; XM_024447084; XM_047421413; XM_017013155; XM_017013160; XM_017013164; NM_001363720 |
| UPK2 | NM_006760 |
| UPK1A | NM_007000; NM_001281443 |
| TM4SF19 | NM_001204898; NM_001204897; NM_138461 |
| SERPINB 13 | NM_001348267; XM_011526029; NM_001348268; NM_012397; NM_001348269; NM_001307923; NM_001348270 |
| SERPINB3 | NM_006919 |
| SERPINB4 | NM_175041; NM_002974; XM_011526138 |
| SPRR2F | NM_001014450; NM_001382255 |
| SPRR2E | NM_001024209 |
| SPRR2A | NM_005988 |
| SPRR2D | NM_001382248; NM_006945 |
| KRT17 | NM_000422 |
| KRT16 | NM_005557 |
| KRT14 | NM_000526 |
| DSG3 | XM_011525850; NM_001944 |
| KRT5 | NM_000424 |
| KRT6C | NM_173086 |
| KRT6A | NM_005554 |
| KRT6B | NM_005555 |
| PLEKHG4B | NM_052909 |
| GNG4 | NM_001098722; XM_047418084; NM_001098721; XM_006711761; XM_011544167; NM_004485 |
| PEG10 | NM_001172438; NM_015068; NM_001172437; NM_001040152; NM_001184961; NM_001184962 |
| SOX2 | NM_003106 |
| TUBB2B | NM_178012 |
| CHGB | NM_001819 |
| SYP | NM_003179 |
| ENO2 | NM_001975 |
| SV2A | NM_001328675; NM_001328674; NM_001278719; NM_014849 |
| MSI1 | XM_011538362; XM_011538361; XM_011538366; XM_047428869; XM_047428871; NM_001414485; NM_001414493; XM_011538370; NM_001414487; NM_001414491; XM_047428870; NM_001414486; NM_001414492; NM_001414495; NM_001414497; NM_002442; XM_011538364; XM_011538371; NM_001414494; NM_001414498; XM_006719403; XM_006719404; XM_011538363; NM_001414488; NM_001414490; XM_011538368; XM_047428872; NM_001414489; NM_001414496 |
| RND2 | XM_011525317; NM_005440; XM_011525316 |
| APLP1 | XM_017026737; NM_005166; NM_001024807; XM_017026738 |
| FGFR3 | XM_047449823; XM_047449824; XM_006713869; XM_006713873; NM_022965; XM_006713868; NM_001354810; XM_011513422; XM_047449821; XM_047449822; NM_000142; XM_011513420; XM_047449820; XM_006713871; XM_006713870; NM_001163213; NM_001354809; NR_148971 |
| TP63 | NM_001114978; NM_001329144; NM_001329146; NM_001329964; NM_001329145; NM_003722; NM_001114979; NM_001114982; NM_001329149; NM_001114980; NM_001114981; NM_001329150; NM_001329148 |
| IRS 1 | NM_005544; XM_047444224; XM_047444223 |
| WNT7B | NM_058238; XM_011530366; NM_001410806 |
| CAPNS2 | NM_032330 |
| ZNF385A | XM_024448935; XM_024448936; NM_001130968; NM_001290002; XM_011538171; NM_001290004; XM_005268783; XM_011538169; XM_047428695; XM_011538168; NM_001290001; XM_011538170; XM_047428696; XM_047428694; NM_001130967; XM_047428697; XM_047428698; NM_015481 |
| SMAD3 | NM_001145103; NM_001145104; NM_001407011; NM_001407015; NM_001407017; NM_001407013; NM_001407016; XM_011521559; NM_001407014; NM_005902; NM_001145102; NM_001407012 |
| SLC2A9 | XM_011513859; XM_011513858; XM_011513866; XM_011513860; XM_011513865; XM_017008460; XM_047415974; NM_020041; XM_011513861; XM_024454151; XM_047415976; NM_001001290; XM_011513864; XM_011513867; XM_017008457; XM_024454153; XM_047415979; XM_011513856; XM_047415978; XM_006713968; XM_047415977; XM_047415980; XM_017008458; XM_024454152; XM_047415973; XM_017008459; XM_047415975 |
| DUOXA1 | XM_011522183; XM_047433288; NM_001276266; NM_001276267; XM_006720746; XM_047433293; NM_001384349; XM_006720752; XM_011522186; XM_047433292; NM_001276264; NM_001276265; XM_006720747; XM_006720743; XM_047433290; XM_006720745; NM_001276268; XM_006720751; XM_011522181; NM_144565; XM_047433289 |
| SYTL1 | XM_047432574; XM_006710990; XM_047432563; NM_001193308; NM_032872; XM_005246022; XM_047432582 |
| SEMA4B | NM_001324030; NM_001324032; NM_001324034; NM_001324031; NM_020210; NM_001393916; NM_001324029; NR_172049; NM_198925 |
| CLCA4 | NR_024602; XM_011541015; NM_012128 |
| PLCH2 | XM_047435038; NM_014638; NM_001303013; XM_047435023; NM_001303012; XM_047435025; XM_047435024; XM_047435029; XM_047435033; XM_047435039; XM_047435028; XM_047435034 |
| SSH3 | NM_017857; XM_047427177; NM_018276 |
| PTPN13 | XM_047416047; NM_080684; XM_017008512; NM_080685; XM_047416041; XM_047416043; XM_011532165; XM_047416040; XM_047416045; XM_047416046; XM_047416048; NM_006264; XM_017008511; XM_017008514; XM_017008515; XM_047416036; XM_047416039; XM_047416042; XM_017008513; XM_047416037; XM_017008516; XM_047416044; NM_080683; XM_047416038 |
| TMPRSS4 | XM_011542901; NM_001290094; XM_005271614; NM_001173552; NR_110734; NM_183247; XM_005271613; XM_011542902; XM_011542904; XM_005271615; NM_001083947; NM_001173551; NM_019894; XM_011542903; XM_047427260; NM_001290096; XM_047427259 |

### EXAMPLES

### Example 1

Since certain cancer therapies, such as cisplatin-based therapeutics and immune checkpoint inhibitors (ICIs), interact with the tumor microenvironment (TME), understanding TME is important to evaluating the efficacy of modern treatments. This example describes identification of urothelial cancer (UC) transcriptomic types that reflect both malignant cell properties and the tumor microenvironment (TME).

In the last decade, UC research has mainly focused on intrinsic features of malignant cells. It has previously been observed that UCs could be characterized according to between 2 and 7 molecular types according to transcriptomic and genomic data, and with different clinicopathological characteristics. However, very few of these previous classifications are based on the tumor microenvironment (TME). As described further below, a urothelial cancer (UC) classification system that assesses both the properties of malignant cells and the tumor microenvironment (TME) was produced.

A meta-cohort of 2418 UC samples from 13 datasets was collected; mutations were identified for 608 samples. The meta-cohort comprised samples from the following databases: GSE124305, GSE87304, GSE128959, GSE83586, GSE70691, GSE48075, GSE13507, GSE69795, GSE32894, GSE154261, GSE133624, and TGCA-BLCA. To estimate the activity of specific genes, urothelial cancer (UC) gene expression signatures were produced from RNA expression data in the datasets. The UC gene expression signatures comprise a gene groups (comprising two or more genes, for example as set forth in Table 1) whose expression or activity are representative of either distinct cell types (e.g., macrophages, tumor infiltrating lymphocytes, etc.), non-cellular components of the TME (e.g., immunosuppressive cytokines, extracellular matrix, etc.), malignant cell biological processes (e.g., proliferation, etc.), and canonical signaling pathway activation (e.g., TGFb, TP53, etc.) in UC.

A total of 24 gene expression signatures relating to immune, stromal and metabolic processes, and four (4) UC-specific gene signatures were used to create a UC TME signature. Examples of the gene groups and genes used to create the UC TME signatures are shown in Table 1. Methods of producing gene group scores for TME signatures are described, for example in International PCT Publication WO2018/231771, published on Dec. 18, 2018, the entire contents of which are herein incorporated by reference).

Gene signatures were produced by performing a single-sample gene set enrichment analysis (ssGSEA) technique using RNA expression data of the genes of each gene group. In some embodiments, the ssGSEA technique is performed according to the following algorithm:

$$s s G S E A s c o r e = \frac{{\sum }_{i}^{N} {r}_{i}^{1.25}}{{\sum }_{i}^{N} {r}_{i}^{0.25}} - \frac{\left(M - N + 1\right)}{2}$$

rᵢ - rank of ith gene in expression matrix
N - number of genes in geneset
M - total number of genes in expression matrix

After UC TME signatures were produced, clustering was performed to identify UC TME types. The clusters were identified in two independent steps. First, a neuroendocrine-like (NE-like) type was identified using a Consensus Classifier (e.g., as described by Kamoun et al., Eur Urol. 2020 Apr;77(4):420-433. doi:10.1016/j.eururo.2019.09.006). A Consensus Classifier was used because the Louvain algorithm cannot identify small clusters, such as the NE-like TME type. Thirty-four tumor samples (1.4%) were identified to be NE-like type and were analyzed separately.

For the rest samples, a Louvain density clustering algorithm was used for community detection (e.g., as described by Blondel et al., J. Stat. Mech. (2008) P10008). For quality control of the clustering following metrics were used: Silhouette score, Calinski-Harabasz score, and Davies-Bouldin score. A schematic depicting one embodiment of a process of generating a UC TME signature using UC gene signatures is shown in FIG. 1.

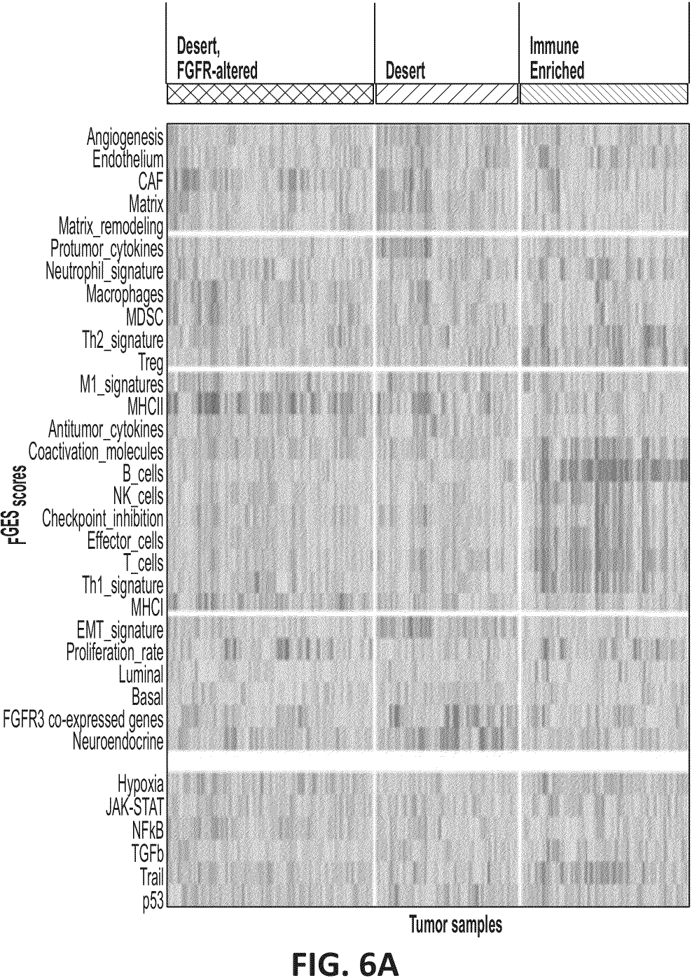

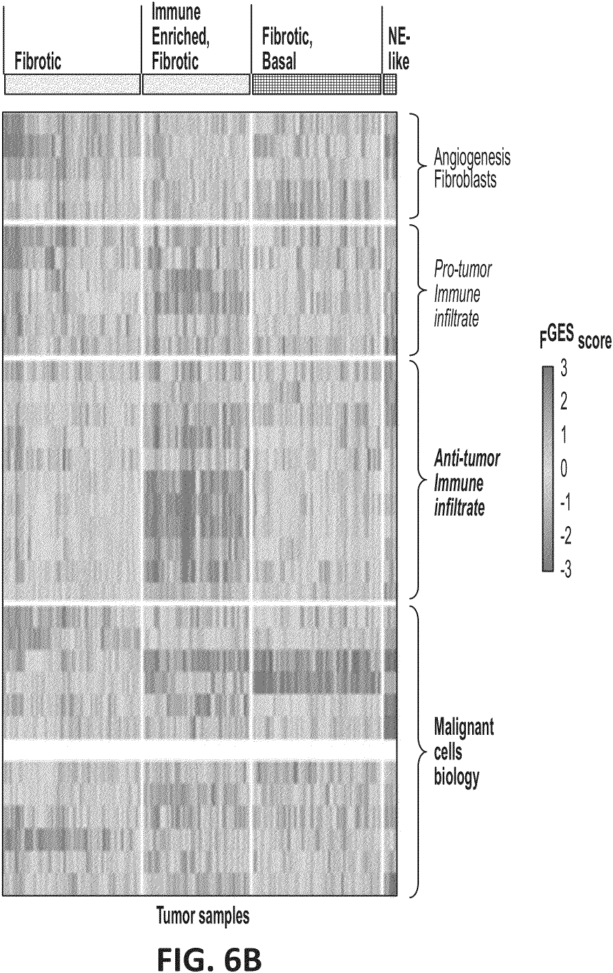

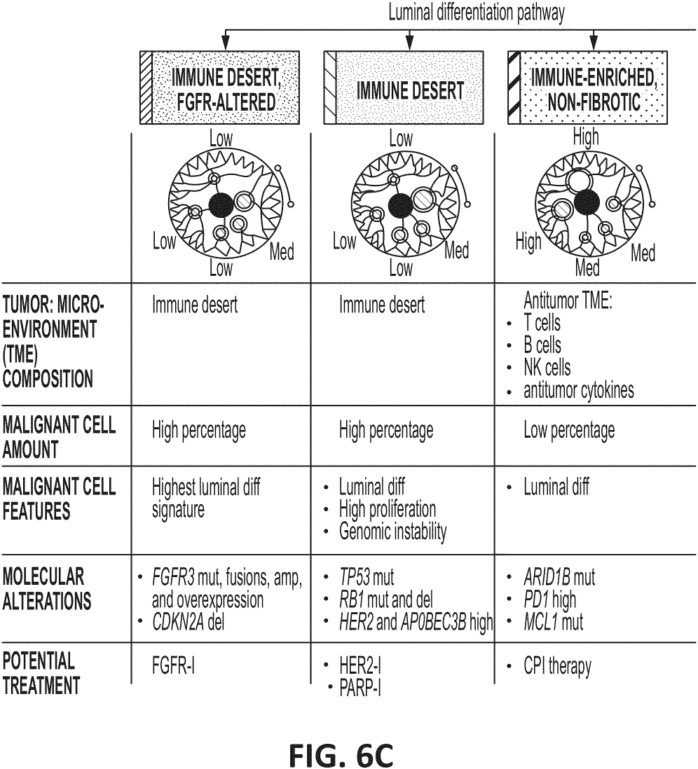

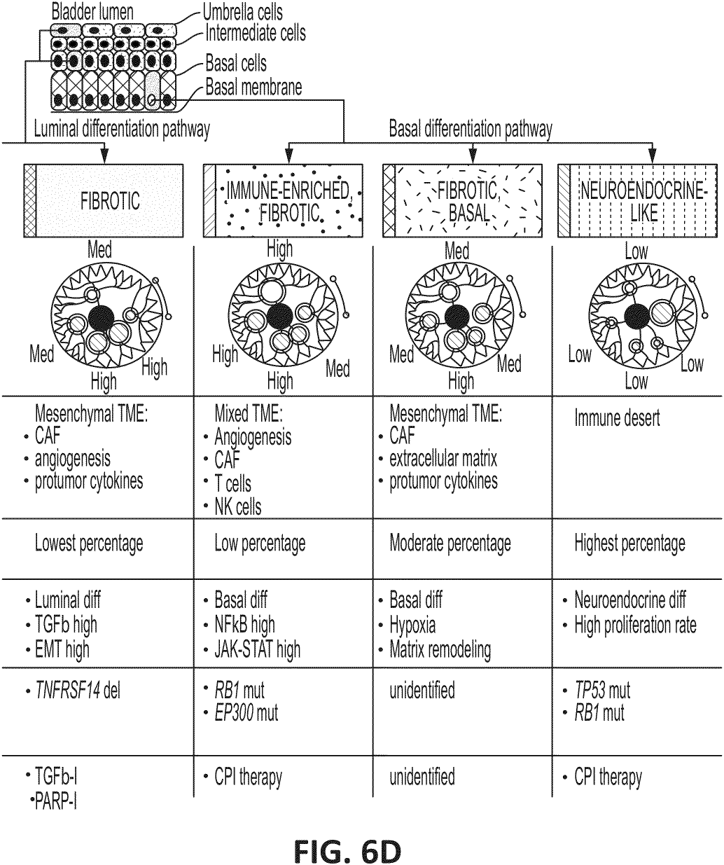

Using unsupervised clustering and consensus classifiers, seven (7) stable UC TME types were identified: Immune Desert (D), Immune Enriched (IE), Fibrotic (F), Immune Enriched -Fibrotic (IE/F), Immune Desert, FGFR-altered (D/FGFR), Fibrotic - Basal (Bas; also referred to as “Basal”), and Neuroendocrine-like (NE). FIG. 6 shows a representative heatmap of urothelial cancer (UC) samples classified into seven distinct UC TME types (D, IE, F, IE/F, D/FGFR, Bas, NE) based on unsupervised dense clustering of 28 gene expression signatures, according to some aspects of the invention. Each column represents one sample.

Below are descriptions of the seven UC types identified by the techniques described in this example.

Immune Desert, FGFR-altered (n=674, 28%) type is characterized by a “desert” TME with an increased proportion of malignant cells with active signature of luminal differentiation, high frequency of FGFR3 mutations (40%), CDKN2A deletions (46%), and high FGFR3 expression. Despite low immune infiltration, patients have a moderate level of ICI response (41%). The UCs of this type predominantly had a papillary phenotype (61%), and the lowest tumor stage and grade.

Immune Desert (n=382, 16%) type is characterized by a “desert” TME with malignant cells that show active signature of luminal differentiation, higher genomic instability, frequent mutations in TP53 and RB1 (69% and 23%), MCL1 amplifications (38%), RB1 deletions (17%), high expression of ERBB2 and APOBEC3B and high proliferation. Patients had a moderate rate of ICI response (42%).

Immune Enriched (n=360, 15%) type is characterized by an “anti-tumor immunity” TME enriched for T-, B- and NK-cells. Malignant cells presented an active signature of luminal differentiation, high frequency of ARID1B mutations (22%), MCL1 amplifications (44%), and high expression of PD1. Patients with this type had the highest ICI response rate (61%) and the best overall survival (OS) rate.

Fibrotic (n=381, 16%) type is characterized by a “mesenchymal” TME enriched for cancer-associated fibroblasts, angiogenesis, endothelium and protumor cytokines. Malignant cells show a high rate of TNFRSF14 deletions (25%), activation of the TGFB signaling and epithelial-to-mesenchymal transition. Fibrotic UCs had the lowest proportion of malignant cells that other types.

Immune Enriched, Fibrotic (n=251, 10%) type is characterized by a “mixed” TME enriched for angiogenesis, macrophages, MDSC, T- and NK-cells. Malignant cells presented an active signature of basal differentiation, high frequency of RB1 and EP300 mutations (28% and 29%), activation of NFkB and JAK-STAT pathways. The UCs of this type were prone to invasion (85%). Patients showed a high response rate (51%) and overall survival (OS) at ICI therapy.

Fibrotic, Basal (n=337, 14%) type is characterized by a “mesenchymal” TME enriched for cancer-associated fibroblasts and extracellular matrix. Malignant cells show the highest activity of basal differentiation signature, and activation of hypoxia and matrix remodeling pathways. Patients had the worst ICI response rate (28%) and worst prognosis for cisplatin-based and ICI therapies.

Neuroendocrine-like (n=33, 1%) type is characterized by a “desert” TME with high proportion of malignant cells with active signature of neuroendocrine differentiation, and tendency to have a high rate of TP53 and RB1 mutations. The UCs of this type showed tendency to invasion (85%), non-papillary histology, high tumor stage and low grade. Patients had the worst OS on cisplatin-based therapy, but the best outcome on ICI therapy (n=4).

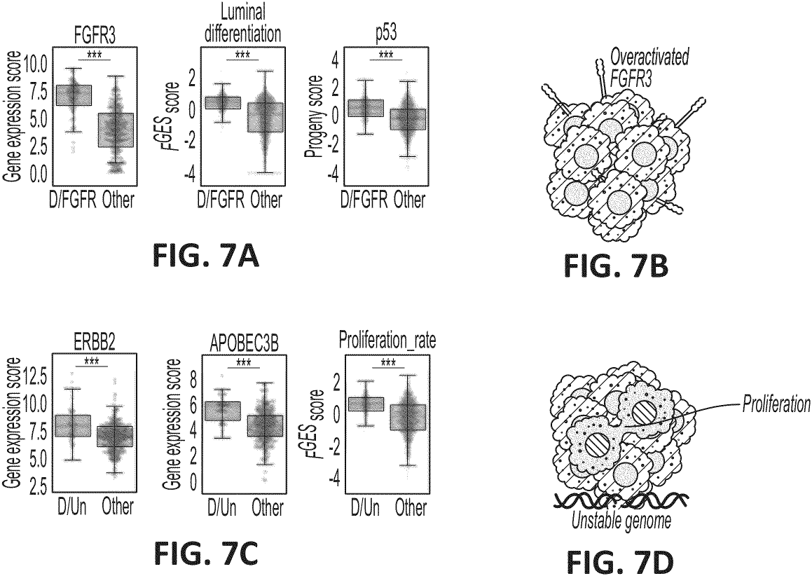

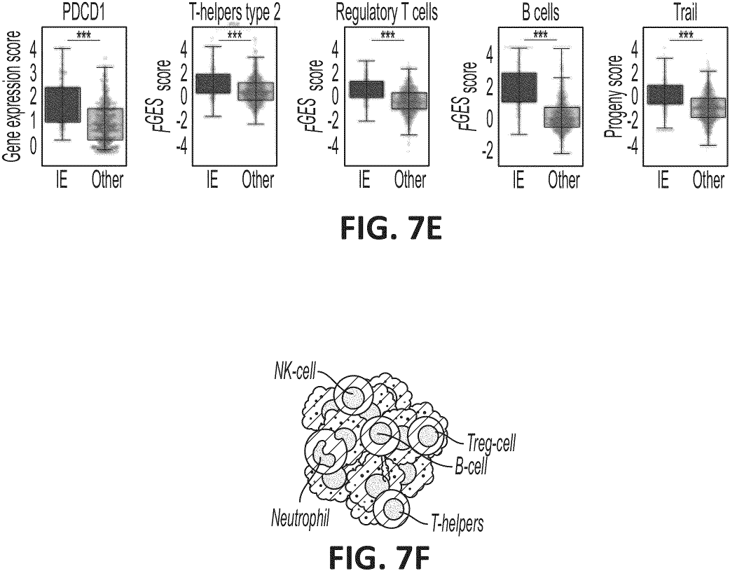

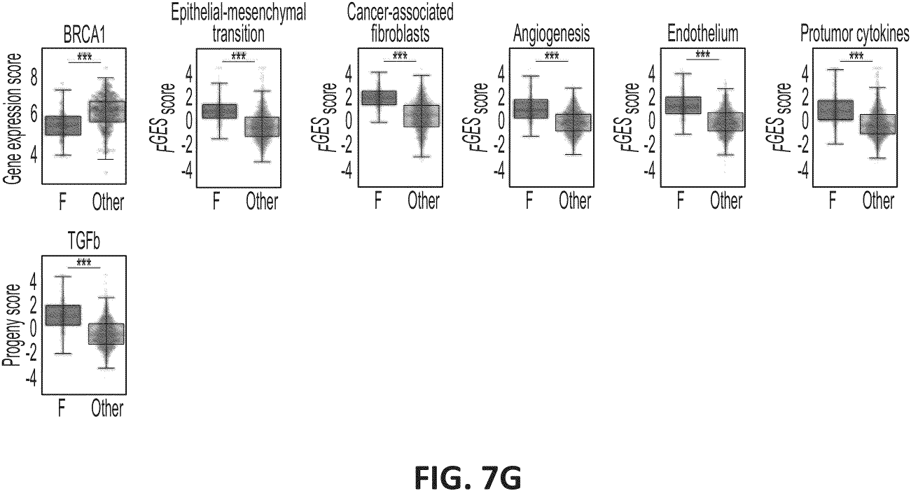

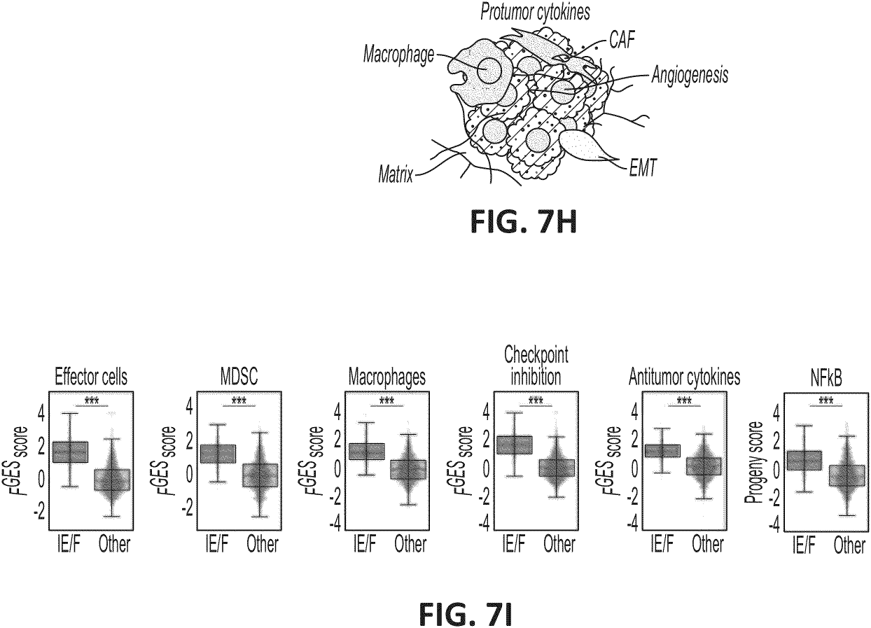

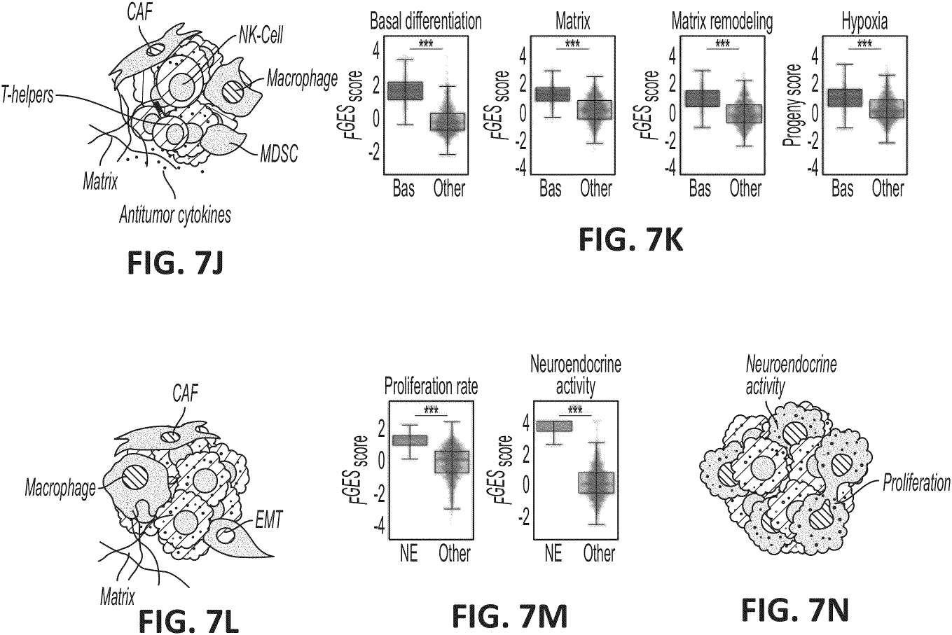

FIGS. 7A-7N show representative data for transcriptomic characterization of UC TME types. FIG. 7A shows gene group scores of the FGRF3, Luminal Differentiation, and p53 gene groups for Desert, FGFR-altered UC TME type. FIG. 7B shows a schematic of Desert, FGFR-altered UC TME having overactivated FGFR3. FIG. 7C shows gene group scores of the ERBB2, APOBEC3B and Proliferation Rate gene groups for Immune Desert (“Desert”) UC TME type. FIG. 7C shows a schematic of Immune Desert UC TME having an unstable genome and high proliferation rate. FIG. 7E shows gene group scores of the PDCD1, T-helper type 2, regulatory T cells, B cells, and Trail gene groups for Immune Enriched UC TME type. FIG. 7F shows a schematic of Immune Enriched UC TME having increased NK cells, neutrophils, B-cells, T-reg cells, and T-helper cells. FIG. 7G shows gene group scores for BRCA1, Epithelial-mesenchymal transition (EMT), Cancer-associated fibroblast (CAF), Angiogenesis, Endothelium, Protumor cytokines, and TGFb gene groups for Fibrotic UC TME type. FIG. 7H shows a schematic of Fibrotic UC TME type having increased protumor cytokines, macrophages, CAF cells, matrix markers, EMT markers, and angiogenesis markers. FIG. 7I shows gene group scores of the Effector cells, MDSC, Macrophages, Checkpoint Inhibition, Antitumor cytokines, and NFkB gene groups for Immune Enriched, Fibrotic UC TME type. FIG. 7J shows a schematic of Immune Enriched, Fibrotic UC TME having increased CAFs, NK cells, macrophages, T helper cells, anti-tumor cytokines, matrix markers, and MDSC markers. FIG. 7K shows gene group scores of Matrix, Matrix remodeling, and Hypoxia gene groups for Basal (also referred to as Fibrotic, Basal) UC TME type. FIG. 7L shows a schematic of Basal UC TME having increased CAFs, macrophages, EMT markers, and matrix markers. FIG. 7M shows gene group scores for Proliferation rate and neuroendocrine activity gene groups of Neuroendocrine-like UC TME type. FIG. 7N shows a schematic of NE-like UC TME having increased neuroendocrine activity and cellular proliferation.

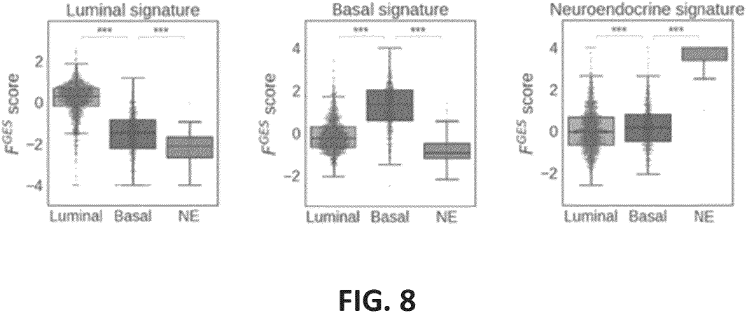

The seven UC TME types were also classified into larger categories of Luminal, Basal, and Neuroendocrine groups. FIG. 8 shows a comparison of UC TME signatures across the three larger groups of UC TME types. The Luminal group includes Desert - FGFR-altered (D/FGFR), Desert (D), Immune Enriched (IE), and Fibrotic (F) UC TME types. The Basal group includes Immune Enriched - Fibrotic (IE/F), and Basal (Bas; also referred to as “Fibrotic Basal”) UC TME types. The Neuroendocrine group consists of the Neuroendocrine-like (NE) UC TME type.

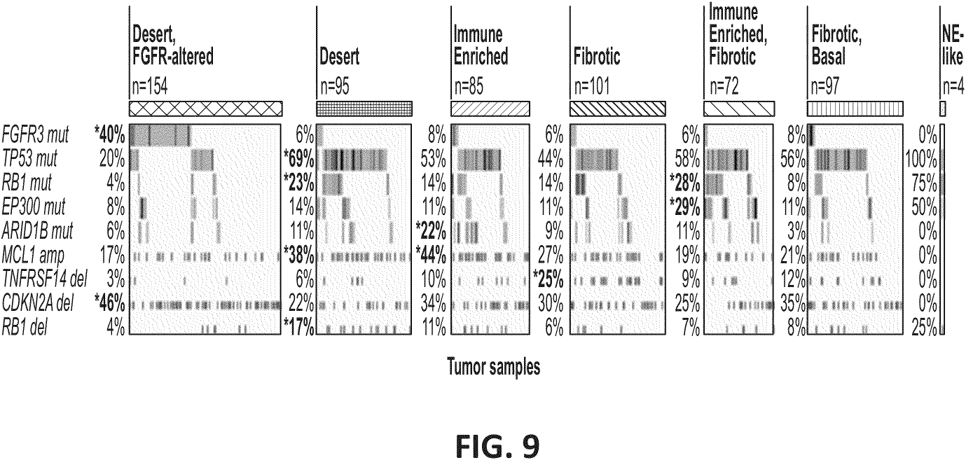

Analysis of genetic mutations associated with each UC TME type was conducted. FIG. 9 shows a representative oncoplot indicating each UC TME type is associated with specific mutations and copy number alterations (CNA). For example, the Desert, FGFR3-altered UC TME type is associated with mutations in FGFR3 and TP53. The Desert UC TME type is associated with mutations in TP53 and RB1, MCL1 amplification, and/or deletion of RB1. The Immune Enriched UC TME type is associated with ARID1B mutations, and amplification of MCL1. The Fibrotic UC TME type is associated with deletion of TNFRSF14. The Immune Enriched, Fibrotic UC TME type is associated with mutations in RB1 and EP300.

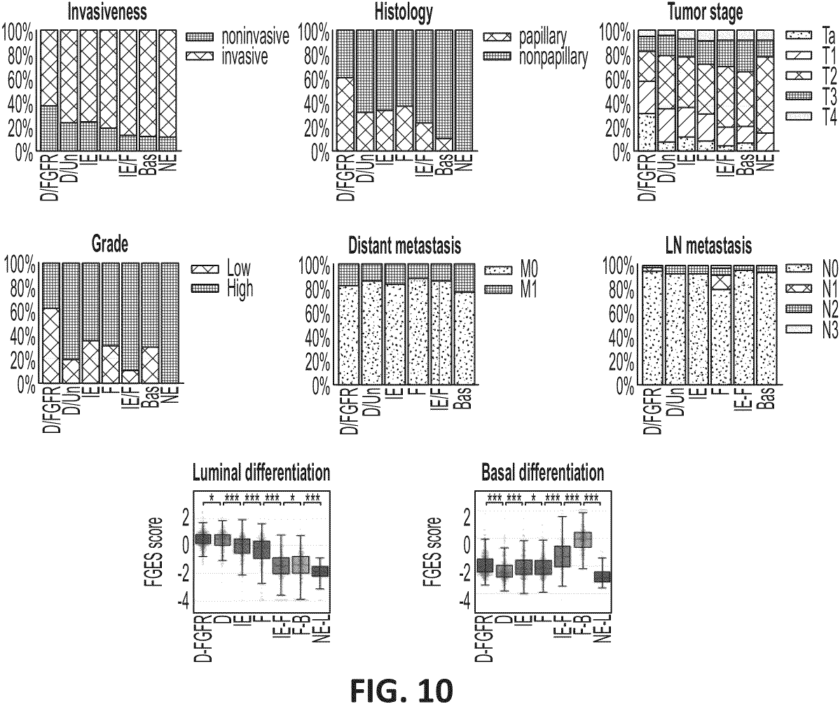

Histopathological patterns associated with UC TME types were also investigated (FIG. 10). Data indicate that Desert FGFR3-altered UC TME type are characterized by increased invasiveness and papillary histology relative to other UC TME types. Data also indicate that Neuroendocrine-like UC TME type is characterized as having the highest level of T2 tumor stage samples relative to other UC TME types. It was also observed that NE and IE/F UC TME types have increased proportions of high grade cancers relative to other UC TME types. However, levels of Distant metastasis (M0, M1) or Lymph node (LN) metastasis (N0, N1, N2, N3) were not observed to vary widely among UC TME types. Differences in Luminal differentiation and Basal differentiation were observed between UC TME types.

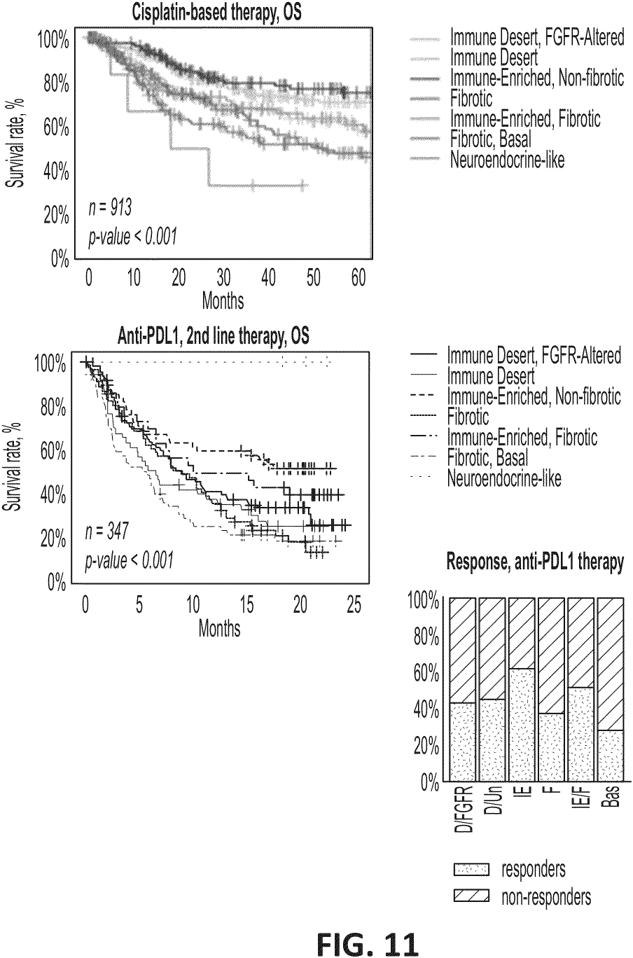

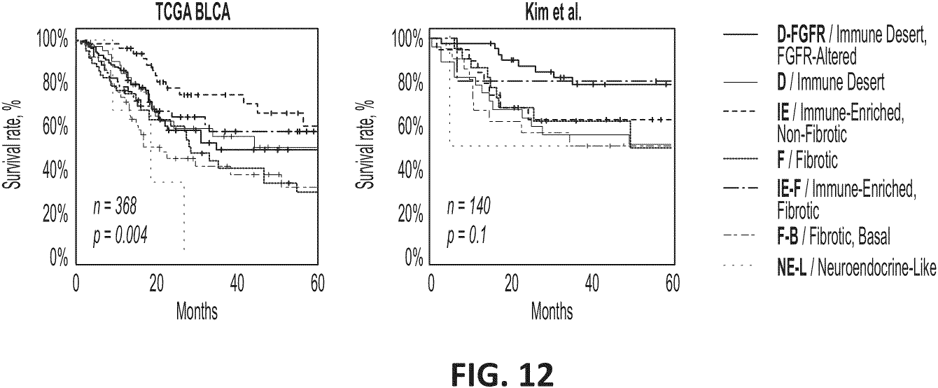

Overall survival (OS) rate to cisplatin-based therapy and anti-PD-L1 second-line therapy, and response rate to anti-PD-L1 therapy were calculated. FIG. 11 shows data indicating subjects having NE-like UC TME had the lowest OS for cisplatin-based therapy but the highest OS for anti-PD-L1 2^{nd} line therapy when seven datasets were combined and analyzed. FIG. 12 shows overall survival (OS) rate for cisplatin-based treatment across UC TME types in the TCGA BLCA dataset (left) and the GSE13507 dataset (right).

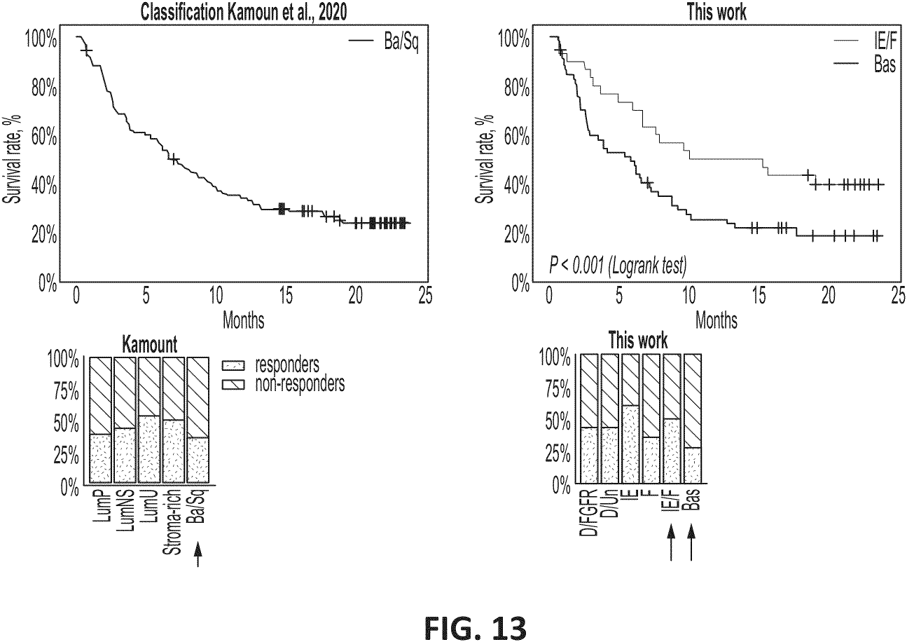

Besides providing in-depth understanding of the tumor processes in urothelial carcinoma, the UC TME typing system described by the disclosure stratifies patients better than previous classifications. Previous techniques subdivided UCs into six classes - luminal papillary, luminal non-specified, luminal unstable, NE-like, stroma-rich and basal-squamous (see, e.g., Kamoun et al., European Urology, 77(4), 2020, 420-433; doi.org/10.1016/j.eururo.2019.09.006). However, using techniques described herein, the previously identified basal-squamous (BalSq) group was split into two novel UC TME types: Immune Enriched - Fibrotic, and Fibrotic - Basal. These novel UC TME types better predict overall survival rate and response rate under atezolizumab therapy, an anti-PDL1 agent than the previously-described classification technique (FIG. 13).

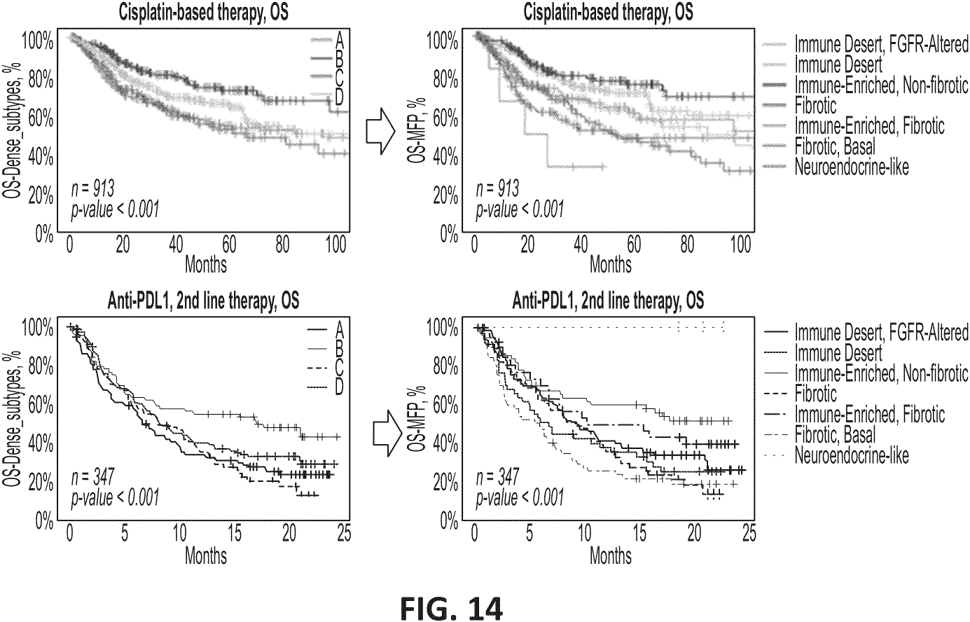

UC TME type classification was also compared to a previously described classical molecular functional (MF) portrait techniques (e.g., as described in PCT/US2018/037017, filed Jun. 12, 2018, published as International Publication No. WO 2018/231771, the entire contents of which are incorporated herein by reference). Novel UC TME types better stratify UC patients and predict overall survival rate under cisplatin-based therapy and anti-PDL1 therapy (FIG. 14) in this context as well.

### Example 2

This example describes selection of therapeutic agents based upon UC TME type.

The Desert, FGFR-altered UC TME type is characterized by a hyperactivated FGFR3 axis, which can be caused by an activating mutation, amplification, fusion, or overexpression of the gene. In some embodiments, Desert, FGFR-altered type patients are suitable targets for Anti-FGFR therapy, which was recently approved by the FDA.

The Desert UC TME type is characterized by many copy number alterations (CNAs) and mutations in ERBB2 and APOBEC3B. ERBB2 is a potential target for therapy, and is now being targeted for the treatment of HER2-positive breast cancer. A large number of genomic rearrangements present in this UC TME type are also targets for PARP inhibitors. In some embodiments, Desert type patients are suitable targets for ERBB2-targeting therapy or PARP inhibitors.

The Immune Enriched UC TME type is characterized by a high content of T-cells and B-cells, and may respond best to immune checkpoint inhibitors (ICI). In some embodiments, Immune Enriched type patients are suitable targets for ICI therapies, for example PD-1 inhibitors, PD-L1 inhibitors, or CTLA-4 inhibitors.

The Fibrotic UC TME type is characterized by a high activity of the stromal component and the TGFb pathway, and may be a target for the TGFb-inhibitors, which can change the tumor microenvironment (TME) from Pro-tumor to Anti-tumor. This UC TME type is also characterized by low activity of DNA damage repair genes or mutations in these genes, in particular in BRCA1, can be targeted by PARP inhibitors. In some embodiments, Fibrotic type patients are suitable targets for TGFb-inhibitors or PARP inhibitors.

The Immune Enriched, Fibrotic UC TME type is characterized by high activity of T-cells and NK-cells, and has a high response rate to ICIs. In some embodiments, Immune Enriched, Fibrotic type patients are suitable targets for ICI therapies.

The Fibrotic, Basal UC TME type is characterized by very high-risk of disease progression and treatment resistance. In some embodiments, Fibrotic, Basal type patients are suitable targets for aggressive treatments, including radiotherapy and chemotherapy, early in the course of disease.

The Neuroendocrine-like UC TME type is characterized by a high response rate to ICI therapy, and the best overall survival (OS) for atezolizumab treatment of all UC TME types. In some embodiments, Neuroendocrine-like type patients are suitable targets for ICI therapies, such as atezolizumab.

### Example 3

Urothelial cancer genomic subtypes based on somatic mutations in cancer driver genes may provide important prognostic and treatment information. Previously, cancer subtypes based on somatic mutations in driver genes clustering were reported for urinary bladder urothelial carcinoma (UBUC) and upper tract urothelial carcinoma (UTUC). Significant differences in five-year survival rates were observed between these subtypes. However, these studies were mainly focused on cancer biology without clinical applications. This example describes identification of clinically relevant UC mutational subtypes based on driver mutations by concurrent use of two classifications (e.g., TME types and genetic subtypes).

To reveal mutational subtype clusters, an algorithm based on a non-negative matrix factorization (NMF) approach was used to analyze two datasets for which whole exome sequencing (WES) was available. At the first step, filtration of mutations was performed based on the following: a mutation was detected in at least four reads, the mutation variant allele frequency was at least 4%, and the mutation type is deleterious (e.g., missense, nonsense, frameshift, or indel mutations). Tumor mutational burden (TMB) was calculated as the number of mutations per megabase. Samples with TMB > 20 were designated as “hypermutated” and analyzed as a separate hypermutated (HM) cluster. For other samples, additional filtration of mutations was performed to retain only driver mutations in cancer driver genes. Cancer driver genes and cancer driver mutations were downloaded from the OncoVar database (oncovar.org/welcome/download). At the final step, only genes mutated in > 20 samples remained. Cases with HRAS/KRAS/NRAS mutations were combined into a single category “RAS”. After all the filtering, mutations remained in the 15 following genes or gene groups: ERCC2, FGFR3, PIK3CA, ARID1A, ATM, CDKN1A, CREBBP, FAT1, FBXW7, KDM6A, RB1, RHOB, TP53, TSC1, RAS (comprising HRAS, KRAS, and NRAS).

Clusterization was performed in two steps. At the first step, the number of clusters was detected using the Hierarchical Dirichlet Process approach from the R package hdp, which is available on GitHub (github.com/nicolaroberts/hdp). It was observed that the optimal number of clusters in the dataset is four (4). Then, clusterization was performed using the publicly available CoGAPS R package (github.com/FertigLab/CoGAPS) with the following parameters: nIterations = 10000, sparseOptimization = TRUE, nPatterns = 4,seed = 105.

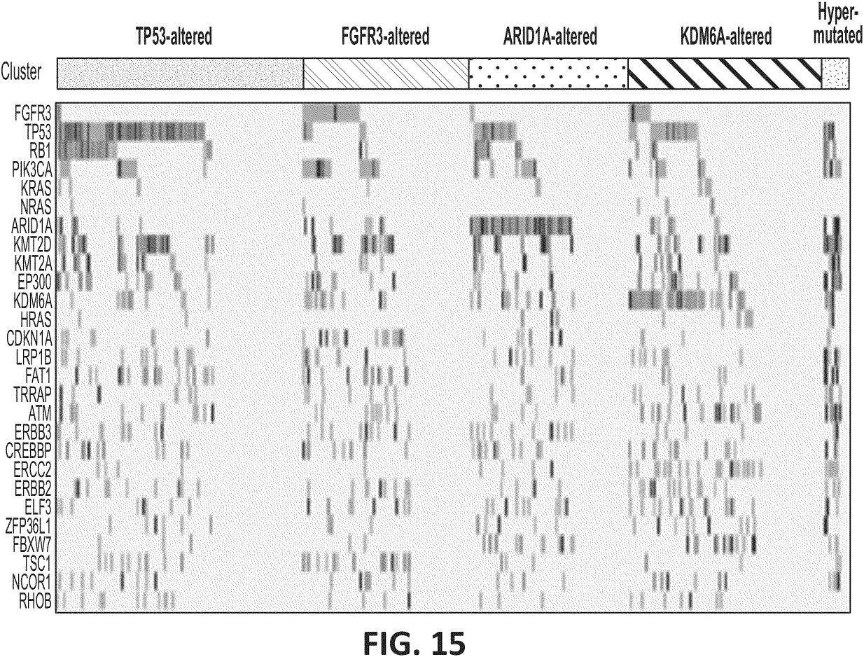

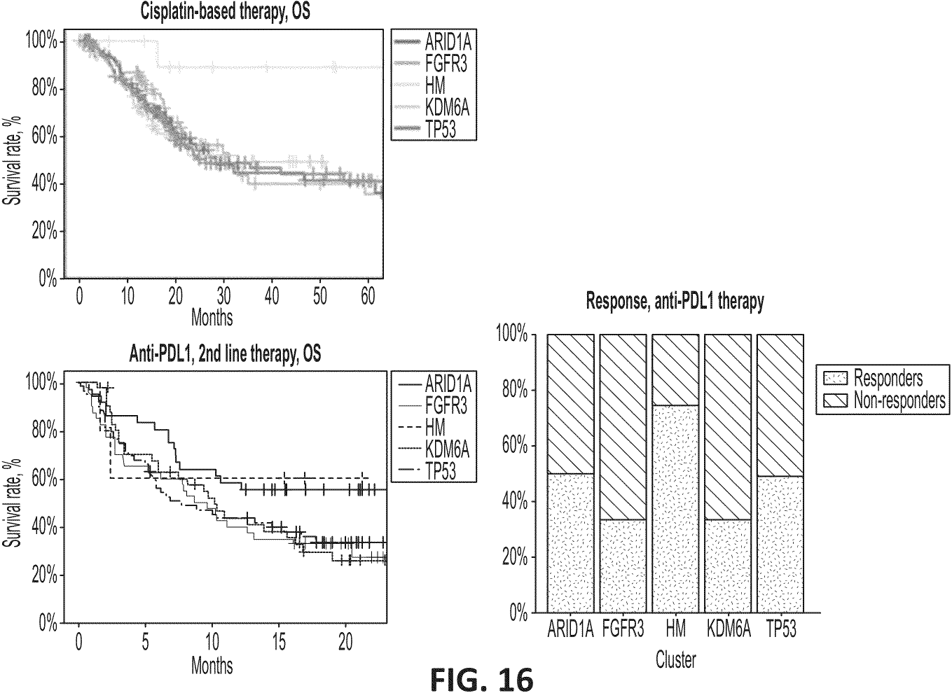

Using the NMF approach four (4) clusters were identified: TP53-altered, KDM6A-altered, FGFR3-altered, and ARIDIA-altered. A fifth cluster, Hypermutated (“HM”), was identified based on the tumor mutational burden (TMB) before clustering (FIG. 15). A schematic depicting one embodiment of a process of generating a UC Mutational Subtype TME signature using UC gene signatures is shown in FIG. 5. Overall survival rate and ICI response across mutational subtypes are shown in FIG. 16. Data indicate that the FGFR3-altered mutational subtype has the highest OS rate in response to cisplatin-based therapy.

TP53-altered (n=162, 31%) subtype is characterized by frequent mutations in TP53 and RB1 genes. Patients had a moderate rate of ICI response (51%) and a low overall survival rate.

KDM6A-altered (n=128, 25%) subtype is characterized by frequent mutations in the KDM6A gene. Patients with KDM6A-altered subtype had a relatively low rate of ICI response (33%) and low overall survival rate.

FGFR3-altered (n=110, 21%) subtype is characterized by frequent mutations in FGFR3 and PIK3CA genes. Patients with FGFR3-altered subtype are potential candidates for anti-FGFR3 therapy, had a relatively low rate of ICI response (33%) and a low overall survival rate.

ARID1A-altered (n=104, 20%) subtype is characterized by frequent mutations in the ARID1A gene. Patients with this subtype had a high overall survival rate on anti-PDL1 therapy and moderate overall survival rate on cisplatin-based therapy.

Hypermutated (n=16, 3%) subtype is characterized by high mutational burden (more than 20 mutations per megabase). Patients with hypermutated subtype had the highest overall survival rate and highest response to ICI therapy (80%).

### EQUIVALENTS

Having thus described several aspects and embodiments of the technology set forth in the disclosure, it is to be appreciated that various alterations, modifications, and improvements will readily occur to those skilled in the art. Such alterations, modifications, and improvements are intended to be within the spirit and scope of the technology described herein. For example, those of ordinary skill in the art will readily envision a variety of other means and/or structures for performing the function and/or obtaining the results and/or one or more of the advantages described herein, and each of such variations and/or modifications is deemed to be within the scope of the embodiments described herein. Those skilled in the art will recognize or be able to ascertain using no more than routine experimentation many equivalents to the specific embodiments described herein. It is, therefore, to be understood that the foregoing embodiments are presented by way of example only and that, within the scope of the appended claims and equivalents thereto, inventive embodiments may be practiced otherwise than as specifically described. In addition, any combination of two or more features, systems, articles, materials, kits, and/or methods described herein, if such features, systems, articles, materials, kits, and/or methods are not mutually inconsistent, is included within the scope of the present disclosure.

The above-described embodiments can be implemented in any of numerous ways. One or more aspects and embodiments of the present disclosure involving the performance of processes or methods may utilize program instructions executable by a device (e.g., a computer, a processor, or other device) to perform, or control performance of, the processes or methods. In this respect, various inventive concepts may be embodied as a computer readable storage medium (or multiple computer readable storage media) (e.g., a computer memory, one or more floppy discs, compact discs, optical discs, magnetic tapes, flash memories, circuit configurations in Field Programmable Gate Arrays or other semiconductor devices, or other tangible computer storage medium) encoded with one or more programs that, when executed on one or more computers or other processors, perform methods that implement one or more of the various embodiments described above. The computer readable medium or media can be transportable, such that the program or programs stored thereon can be loaded onto one or more different computers or other processors to implement various ones of the aspects described above. In some embodiments, computer readable media may be non-transitory media.

The terms “program” or “software” are used herein in a generic sense to refer to any type of computer code or set of computer-executable instructions that can be employed to program a computer or other processor to implement various aspects as described above. Additionally, it should be appreciated that according to one aspect, one or more computer programs that when executed perform methods of the present disclosure need not reside on a single computer or processor, but may be distributed in a modular fashion among a number of different computers or processors to implement various aspects of the present disclosure.

Computer-executable instructions may be in many forms, such as program modules, executed by one or more computers or other devices. Generally, program modules include routines, programs, objects, components, data structures, etc. that perform particular tasks or implement particular abstract data types. Typically the functionality of the program modules may be combined or distributed as desired in various embodiments.

Also, data structures may be stored in computer-readable media in any suitable form. For simplicity of illustration, data structures may be shown to have fields that are related through location in the data structure. Such relationships may likewise be achieved by assigning storage for the fields with locations in a computer-readable medium that convey relationship between the fields. However, any suitable mechanism may be used to establish a relationship between information in fields of a data structure, including through the use of pointers, tags or other mechanisms that establish relationship between data elements.

When implemented in software, the software code can be executed on any suitable processor or collection of processors, whether provided in a single computer or distributed among multiple computers.

Further, it should be appreciated that a computer may be embodied in any of a number of forms, such as a rack-mounted computer, a desktop computer, a laptop computer, or a tablet computer, as non-limiting examples. Additionally, a computer may be embedded in a device not generally regarded as a computer but with suitable processing capabilities, including a Personal Digital Assistant (PDA), a smartphone, a tablet, or any other suitable portable or fixed electronic device.

Also, a computer may have one or more input and output devices. These devices can be used, among other things, to present a user interface. Examples of output devices that can be used to provide a user interface include printers or display screens for visual presentation of output and speakers or other sound generating devices for audible presentation of output. Examples of input devices that can be used for a user interface include keyboards, and pointing devices, such as mice, touch pads, and digitizing tablets. As another example, a computer may receive input information through speech recognition or in other audible formats.

Such computers may be interconnected by one or more networks in any suitable form, including a local area network or a wide area network, such as an enterprise network, and intelligent network (IN) or the Internet. Such networks may be based on any suitable technology and may operate according to any suitable protocol and may include wireless networks, wired networks or fiber optic networks.

Also, as described, some aspects may be embodied as one or more methods. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B,” when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semiclosed transitional phrases, respectively.

The terms “approximately,” “substantially,” and “about” may be used to mean within ±20% of a target value in some embodiments, within ±10% of a target value in some embodiments, within ±5% of a target value in some embodiments, within ±2% of a target value in some embodiments. The terms “approximately,” “substantially,” and “about” may include the target value.

What is claimed is:

1. A method for determining a urothelial cancer (UC) tumor microenvironment (TME) type of a subject having, suspected of having, or at risk of having a urothelial cancer, the method comprising:
using at least one computer hardware processor to perform:
obtaining RNA expression data for the subject, the RNA expression data indicating RNA expression levels for at least some genes in each group of at least some of a plurality of gene groups listed in Table 1;
generating a UC TME signature for the subject using the RNA expression data, the UC TME signature comprising gene group scores for respective gene groups in the at least some of the plurality of gene groups, the generating comprising:
determining the gene group scores using the RNA expression levels; and
identifying, using the UC TME signature and from among a plurality of UC TME types, a UC TME type for the subject.

2. The method of claim 1, further comprising:
normalizing the RNA expression data to transcripts per million (TPM) units prior to generating the UC TME signature.

3. The method of claim 1, wherein the RNA expression levels comprise RNA expression levels for at least three genes from each of at least two of the following gene groups:
Luminal differentiation group: PWRN1, PWRN3, GSTM5, GSTM4, GSTM2, ZNF321P, ZNF320, ZNF66, ZNF737, KRT20, UPK1B, FOXA1, ACER2, SEMA5A, PPARG, GATA3, SNX31, UPK2, UPK1A;
Basal differentiation group: TM4SF19, SERPINB13, SERPINB3, SERPINB4, SPRR2F, SPRR2E, SPRR2A, SPRR2D, KRT17, KRT16, KRT14, DSG3, KRT5, KRT6C, KRT6A, KRT6B;
Neuroendocrine differentiation group: PLEKHG4B, GNG4, PEG10, SOX2, TUBB2B, CHGB, SYP, ENO2, SV2A, MSI1, RND2, APLP1; and
FGFR3 co-expressed group: FGFR3, TP63, IRS1, WNT7B, CAPNS2, ZNF385A, SMAD3, SLC2A9, DUOXA1, SYTL1, SEMA4B, CLCA4, PLCH2, SSH3, PTPN13, TMPRSS4.

4. The method of claim 1, wherein determining the gene group scores comprises:
determining a respective gene group score for each of at least two of the following gene groups, using, for a particular gene group, RNA expression levels for at least three genes in the particular gene group to determine the gene group score for the particular group, the gene groups including:
Luminal differentiation group: PWRN1, PWRN3, GSTM5, GSTM4, GSTM2, ZNF321P, ZNF320, ZNF66, ZNF737, KRT20, UPK1B, FOXA1, ACER2, SEMA5A, PPARG, GATA3, SNX31, UPK2, UPK1A;
Basal differentiation group: TM4SF19, SERPINB13, SERPINB3, SERPINB4, SPRR2F, SPRR2E, SPRR2A, SPRR2D, KRT17, KRT16, KRT14, DSG3, KRT5, KRT6C, KRT6A, KRT6B;
Neuroendocrine differentiation group: PLEKHG4B, GNG4, PEG10, SOX2, TUBB2B, CHGB, SYP, ENO2, SV2A, MSI1, RND2, APLP1; and
FGFR3 co-expressed group: FGFR3, TP63, IRS1, WNT7B, CAPNS2, ZNF385A, SMAD3, SLC2A9, DUOXA1, SYTL1, SEMA4B, CLCA4, PLCH2, SSH3, PTPN13, TMPRSS4.

5. The method of claim 4, wherein the determining the gene group scores further comprises determining a respective gene group score for each of at least two of the following gene groups, using, for a particular gene group, RNA expression levels for at least three genes in the particular gene group to determine the gene group score for the particular group, the gene groups including:
(a) MHC type I group: HLA-C, TAPBP, HLA-B, B2M, TAP2, HLA-A, TAP1, NLRC5;
(b) MHC type II group: HLA-DQB1, HLA-DMA, HLA-DMB, HLA-DRA, CIITA, HLA-DQA1, HLA-DPB1, HLA-DRB1, HLA-DPA1;
(c) Coactivation molecules group: TNFRSF4, CD27, CD80, CD40LG, TNFRSF9, CD40, CD28, ICOSLG, CD83, TNFSF9, CD70, TNFSF4, ICOS, CD86;
(d) Effector cells group: ZAP70, GZMB, GZMK, IFNG, FASLG, EOMES, TBX21, GZMA, CD8A, GNLY, PRF1, CD8B;
(e) Natural killer cells group: NKG7, FGFBP2, CD244, KLRK1, KIR2DL4, CD226, KLRF1, GNLY, GZMB, KLRC2, NCR1, GZMH, IFNG, SH2D1B, NCR3, EOMES, CD160;
(f) T cells group: TRBC2, CD3E, CD3G, ITK, CD28, TRBC1, TRAT1, TBX21, CD5, TRAC, CD3D;
(g) T-helper cells type 1 group: IL21, TBX21, IL12RB2, CD40LG, IFNG, IL2, STAT4;
(h) T-helper cells type 2 group: IL13, CCR4, IL10, IL4, IL5;
(i) B cells group: CD22, TNFRSF13C, STAP1, CD79B, PAX5, CR2, TNFRSF13B, CD79A, TNFRSF17, FCRL5, MS4A1, CD19, BLK;
(j) Macrophages group: MRC1, SIGLEC1, MSR1, CD163, CSF1R, CD68, IL4I1, IL10;
(k) Macrophages type 1 group: CMKLR1, SOCS3, IRF5, NOS2, IL1B, IL12B, IL23A, TNF, IL12A;
(1) Antitumor cytokines group: CCL3, IL21, IFNB1, IFNA2, TNF, TNFSF10;
(m) Checkpoint inhibition group: PDCD1, BTLA, HAVCR2, CD274, VSIR, LAG3, TIGIT, PDCD1LG2, CTLA4;
(n) T-regulatory cells group: IKZF2, TNFRSF18, IL10, FOXP3, CCR8, IKZF4, CTLA4;
(o) Neutrophils group: CD177, FFAR2, PGLYRP1, CXCR1, MPO, CXCR2, ELANE, CTSG, PRTN3, FCGR3B;
(p) MDSC group: ARG1, IL6, CYBB, IL10, PTGS2, IDO1, IL4I1;
(q) Protumor cytokines group: TGFB2, MIF, IL6, TGFB3, TGFB1, IL22, IL10;
(r) Cancer associated fibroblasts (CAF) group: COL6A3, PDGFRB, COL6A1, MFAP5, COL5A1, FAP, PDGFRA, FGF2, ACTA2, COL6A2, FBLN1, CD248, COL1A1, MMP2, COL1A2, MMP3, LUM, CXCL12, LRP1;
(s) Matrix group: LAMC2, TNC, COL11A1, VTN, LAMB3, COL1A1, FN1, LAMA3, LGALS9, COL1A2, COL4A1, COL5A1, ELN, LGALS7, COL3A1;
(t) Matrix remodeling group: ADAMTS4, ADAMTS5, CA9, LOX, MMP1, MMP11, MMP12, MMP2, MMP3, MMP7, MMP9, PLOD2;
(u) Angiogenesis group: VEGFC, VEGFA, PDGFC, KDR, CDH5, VEGFB, PGF, TEK, ANGPT2, CXCR2, FLT1, CXCL8, VWF, ANGPT1, CXCL5;
(v) Endothelium group: KDR, CDH5, NOS3, VCAM1, VWF, FLT1, MMRN1, CLEC14A, ENG, MMRN2;
(w) Proliferation_rate group: CCND1, CCNB1, CETN3, CDK2, E2F1, AURKA, BUB1, AURKB, PLK1, MCM6, ESCO2, MYBL2, MKI67, MCM2, CCNE1; and
(x) Epithelial to mesenchymal transition group: CDH2, ZEB1, ZEB2, TWIST1, SNAI1, SNAI2, TWIST2.

6. The method of claim 1, wherein determining the gene group scores comprises:
determining a respective gene group score for each of the following gene groups, using, for a particular gene group, RNA expression levels for all genes in the particular gene group to determine the gene group score for the particular group, the gene groups including:
Luminal differentiation group: PWRN1, PWRN3, GSTM5, GSTM4, GSTM2, ZNF321P, ZNF320, ZNF66, ZNF737, KRT20, UPK1B, FOXA1, ACER2, SEMA5A, PPARG, GATA3, SNX31, UPK2, UPK1A;
Basal differentiation group: TM4SF19, SERPINB13, SERPINB3, SERPINB4, SPRR2F, SPRR2E, SPRR2A, SPRR2D, KRT17, KRT16, KRT14, DSG3, KRT5, KRT6C, KRT6A, KRT6B;
Neuroendocrine differentiation group: PLEKHG4B, GNG4, PEG10, SOX2, TUBB2B, CHGB, SYP, ENO2, SV2A, MSI1, RND2, APLP1; and
FGFR3 co-expressed group: FGFR3, TP63, IRS1, WNT7B, CAPNS2, ZNF385A, SMAD3, SLC2A9, DUOXA1, SYTL1, SEMA4B, CLCA4, PLCH2, SSH3, PTPN13, TMPRSS4.

7. The method of any claim 6, wherein determining the gene group scores further comprises:
determining a respective gene group score for each of the following gene groups, using, for a particular gene group, RNA expression levels for all genes in the particular gene group to determine the gene group score for the particular group, the gene groups including:
(a) MHC type I group: HLA-C, TAPBP, HLA-B, B2M, TAP2, HLA-A, TAP1, NLRC5;
(b) MHC type II group: HLA-DQB1, HLA-DMA, HLA-DMB, HLA-DRA, CIITA, HLA-DQA1, HLA-DPB1, HLA-DRB1, HLA-DPA1;
(c) Coactivation molecules group: TNFRSF4, CD27, CD80, CD40LG, TNFRSF9, CD40, CD28, ICOSLG, CD83, TNFSF9, CD70, TNFSF4, ICOS, CD86;
(d) Effector cells group: ZAP70, GZMB, GZMK, IFNG, FASLG, EOMES, TBX21, GZMA, CD8A, GNLY, PRF1, CD8B;
(e) Natural killer cells group: NKG7, FGFBP2, CD244, KLRK1, KIR2DL4, CD226, KLRF1, GNLY, GZMB, KLRC2, NCR1, GZMH, IFNG, SH2D1B, NCR3, EOMES, CD160;
(f) T cells group: TRBC2, CD3E, CD3G, ITK, CD28, TRBC1, TRAT1, TBX21, CD5, TRAC, CD3D;
(g) T-helper cells type 1 group: IL21, TBX21, IL12RB2, CD40LG, IFNG, IL2, STAT4;
(h) T-helper cells type 2 group: IL13, CCR4, IL10, IL4, IL5;
(i) B cells group: CD22, TNFRSF13C, STAP1, CD79B, PAX5, CR2, TNFRSF13B, CD79A, TNFRSF17, FCRL5, MS4A1, CD19, BLK;
(j) Macrophages group: MRC1, SIGLEC1, MSR1, CD163, CSF1R, CD68, IL4I1, IL10;
(k) Macrophages type 1 group: CMKLR1, SOCS3, IRF5, NOS2, IL1B, IL12B, IL23A, TNF, IL12A;
(l) Antitumor cytokines group: CCL3, IL21, IFNB1, IFNA2, TNF, TNFSF10;
(m) Checkpoint inhibition group: PDCD1, BTLA, HAVCR2, CD274, VSIR, LAG3, TIGIT, PDCD1LG2, CTLA4;
(n) T-regulatory cells group: IKZF2, TNFRSF18, IL10, FOXP3, CCR8, IKZF4, CTLA4;
(o) Neutrophils group: CD177, FFAR2, PGLYRP1, CXCR1, MPO, CXCR2, ELANE, CTSG, PRTN3, FCGR3B;
(p) MDSC group: ARG1, IL6, CYBB, IL10, PTGS2, IDO1, IL4I1;
(q) Protumor cytokines group: TGFB2, MIF, IL6, TGFB3, TGFB1, IL22, IL10;
(r) Cancer associated fibroblasts (CAF) group: COL6A3, PDGFRB, COL6A1, MFAP5, COL5A1, FAP, PDGFRA, FGF2, ACTA2, COL6A2, FBLN1, CD248, COL1A1, MMP2, COL1A2, MMP3, LUM, CXCL12, LRP1;
(s) Matrix group: LAMC2, TNC, COL11A1, VTN, LAMB3, COL1A1, FN1, LAMA3, LGALS9, COL1A2, COL4A1, COL5A1, ELN, LGALS7, COL3A1;
(t) Matrix remodeling group: ADAMTS4, ADAMTS5, CA9, LOX, MMP1, MMP11, MMP12, MMP2, MMP3, MMP7, MMP9, PLOD2;
(u) Angiogenesis group: VEGFC, VEGFA, PDGFC, KDR, CDH5, VEGFB, PGF, TEK, ANGPT2, CXCR2, FLT1, CXCL8, VWF, ANGPT1, CXCL5;
(v) Endothelium group: KDR, CDH5, NOS3, VCAM1, VWF, FLT1, MMRN1, CLEC14A, ENG, MMRN2;
(w) Proliferation_rate group: CCND1, CCNB1, CETN3, CDK2, E2F1, AURKA, BUB1, AURKB, PLK1, MCM6, ESCO2, MYBL2, MKI67, MCM2, CCNE1; and
(x) Epithelial to mesenchymal transition group: CDH2, ZEB1, ZEB2, TWIST1, SNAI1, SNAI2, TWIST2.

8. The method of claim 1, wherein the plurality of UC TME types is associated with a respective plurality of UC TME signature clusters, wherein identifying, using the UC TME signature and from among a plurality of UC TME types, the UC TME type for the subject comprises:
associating the UC TME signature of the subject with a particular one of the plurality of UC TME signature clusters; and,
identifying the UC TME type for the subject as the UC TME type corresponding to the particular one of the plurality of UC TME signature clusters to which the UC TME signature of the subject is associated.

9. The method of claim 1, wherein the plurality of a plurality of UC TME types comprises: Immune Desert (D) type, Immune Enriched (IE) type, Fibrotic (F) type, Immune Enriched -Fibrotic (IE/F) type, Immune Desert type, FGRF-altered (D/FGFR) type, Fibrotic - Basal (Bas) type, and Neuroendocrine-like (NE) type.

10. The method of claim 9, further comprising
administering an anti-FGFR agent to the subject when the subject is identified as having Desert, FGFR-altered type UC TME;
administering an ERBB2-targeting therapy or PARP inhibitor to the subject when the subject is identified as having Desert type UC TME;
administering an immune checkpoint inhibitor (ICI) to the subject when the subject is identified as having Immune Enriched type UC TME;
administering a candidate for treatment with a TGFb inhibitor or PARP inhibitor to the subject when the subject is identified as having Fibrotic type UC TME;
administering an immune checkpoint inhibitor (ICI) to the subject when the subject is identified as having Immune Enriched, Fibrotic type UC TM; or
administering an immune checkpoint inhibitor (ICI) to the subject when the subject is identified as having Neuroendocrine-like type UC TME.

11. A method for determining a urothelial cancer (UC) mutational subtype of a subject having, suspected of having, or at risk of having a urothelial cancer, the method comprising:
using at least one computer hardware processor to perform:
obtaining RNA expression data for the subject, the RNA expression data indicating RNA expression levels for genes of the subject;
generating a UC mutational subtype signature for the subject using the RNA expression data, the generating comprising:
analyzing the RNA expression data to identify the presence or absence of one or more mutations in the one or more of the following genes: ERCC2, FGFR3, PIK3CA, ARID1A, ATM, CDKN1A, CREBBP, FAT1, FBXW7, KDM6A, RB1, RHOB, TP53, TSC1, HRAS, KRAS, and NRAS; and
identifying, using the UC mutational subtype signature and from among a plurality of UC mutational subtypes, a UC mutational subtype for the subject.

12. The method of claim 11, wherein the plurality of UC mutational subtypes is associated with a respective plurality of UC mutational subtype clusters, wherein identifying, using the UC mutational subtype signature and from among a plurality of UC mutational subtypes, the UC mutational subtype for the subject comprises:
associating the UC mutational subtype signature of the subject with a particular one of the plurality of UC mutational subtype clusters; and,
identifying the UC mutational subtype for the subject as the UC mutational subtype corresponding to the particular one of the plurality of UC mutational subtype clusters to which the UC mutational subtype signature of the subject is associated.

13. The method of claim 11, further comprising generating the plurality of UC mutational subtype clusters, the generating comprising:
obtaining multiple sets of RNA expression data by sequencing biological samples from multiple respective subjects, the RNA each of the multiple sets of expression data indicating RNA expression levels for genes in the subjects;
generating multiple UC mutational subtype signatures from the multiple sets of RNA expression data, the generating comprising, for each particular one of the multiple UC mutational subtype signatures:
analyzing the particular set of RNA expression data for which the particular one UC mutational subtype signature is being generated to identify the presence or absence of one or more mutations in the one or more of the following genes: ERCC2, FGFR3, PIK3CA, ARID1A, ATM, CDKN1A, CREBBP, FAT1, FBXW7, KDM6A, RB1, RHOB, TP53, TSC1, HRAS, KRAS, and NRAS; and
clustering the multiple UC mutational subtype signatures to obtain the plurality of UC mutational subtype clusters.

14. The method of claim 11, wherein the plurality of a plurality of UC mutational subtype clusters comprises: TP53-altered type, KDM6A-altered type, FGFR3-altered type, ARID1A-altered type, and Hypermutated (“HM”) type.

15. The method of claim 11, further comprising identifying the subject as a candidate for treatment with an immune checkpoint inhibitor (ICI) when the subject is identified as having TP53-altered type, ARID1A-altered type, or Hypermutated (“HM”) type UC mutational subtype.

16. The method of claim 15, further comprising
administering an anti-FGFR agent to the subject when the subject is identified as having FGFR3-altered type UC mutational subtype; or
administering cisplatin when the subject is identified as having ARID1A-altered type UC mutational subtype.

17. A system, comprising:
at least one computer hardware processor; and
at least one computer-readable storage medium storing processor-executable instructions that, when executed by the at least one computer hardware processor, cause the at least one computer hardware processor to perform a method for determining a urothelial cancer (UC) tumor microenvironment (TME) type of a subject having, suspected of having, or at risk of having a urothelial cancer, the method comprising:
obtaining RNA expression data for the subject, the RNA expression data indicating RNA expression levels for at least some genes in each group of at least some of a plurality of gene groups listed in Table 1;
generating a UC TME signature for the subject using the RNA expression data, the UC TME signature comprising gene group scores for respective gene groups in the at least some of the plurality of gene groups, the generating comprising:
determining the gene group scores using the RNA expression levels; and
identifying, using the UC TME signature and from among a plurality of UC TME types, a UC TME type for the subject.

18. At least one computer-readable storage medium storing processor-executable instructions that, when executed by at least one computer hardware processor, cause the at least one computer hardware processor to perform the method for determining a urothelial cancer (UC) tumor microenvironment (TME) type of a subject having, suspected of having, or at risk of having a urothelial cancer according to claim 1.

19. A system, comprising:
at least one computer hardware processor; and
at least one computer-readable storage medium storing processor-executable instructions that, when executed by the at least one computer hardware processor, cause the at least one computer hardware processor to perform the method for determining a urothelial cancer (UC) mutational subtype of a subject having, suspected of having, or at risk of having a urothelial cancer according to claim 11.

20. At least one computer-readable storage medium storing processor-executable instructions that, when executed by at least one computer hardware processor, cause the at least one computer hardware processor to perform the method for determining a urothelial cancer (UC) mutational subtype of a subject having, suspected of having, or at risk of having a urothelial cancer according to claim 11.
